(19) United States
(12) Patent Application Publication
(10) Pub. No.: US 2020/0085876 A1
(43) Pub. Date: Mar. 19, 2020

(54) IMMUNOMODULATING CELL CIRCUITS

(71) Applicant: Senti Biosciences, Inc., South San Francisco, CA (US)

(72) Inventors: Timothy Kuan-Ta Lu, Cambridge, MA (US); Russell Morrison Gordley, San Francisco, CA (US); Jack Tzu-Chiao Lin, Redwood City, CA (US); Brian Scott Garrison, San Jose, CA (US); Philip Janmin Lee, Alameda, CA (US)

(21) Appl. No.: 16/494,482

(22) PCT Filed: Mar. 16, 2018
(86) PCT No.: PCT/US18/22855

§ 371 Date: Sep. 16, 2019

Related U.S. Application Data
(60) Provisional application No. 62/473,198, filed on Mar. 17, 2017.

(51) Int. Cl. A61K 35/28; A61K 38/17; A61K 38/20; C12N 5/0775

(52) U.S. Cl. CPC A61K 35/28; A61K 38/1777; A61K 38/2026; A61K 38/2066; C12N 5/0663; C12N 2502/99; C12N 5/0667; C12N 2501/2304; C12N 2501/231; C12N 2502/1114; C12N 5/0665

(57) ABSTRACT

Provided herein are methods and compositions for dynamically controlling and targeting multiple arms of the immune system. Some aspects provide mesenchymal stem cells (MSCs) engineered to produce multiple effector molecules. In some instances, each effector molecule modulates a different cell type of the immune system or different functions of a cell. Also provided herein are methods of using the MSCs to treat or alleviate symptoms of inflammatory bowel disease (IBD), for example.

### RELATED APPLICATION

This application claims the benefit under 35 U.S.C. § 119(e) of U.S. provisional application No. 62/473,198, filed Mar. 17, 2017, which is incorporated by reference herein in its entirety.

### BACKGROUND

The immune system, as a host defense system, protects against disease. The immune system is classified into subsystems, such as the innate immune system and the adaptive immune system, or humoral immunity and cell-mediated immunity. In humans, the blood-brain barrier, blood-cerebrospinal fluid barrier, and similar fluid-brain barriers separate the peripheral immune system from the neuro-immune system, which protects the brain. The immune system protects organisms from infection with layered defenses of increasing specificity. For example, the innate immune system provides an immediate, but non-specific response, while the adaptive immune system, activated by the innate immune system, provides immunological memory. Dysregulation of the immune system underlies a large number of important and difficult-to-treat diseases, such as autoimmune diseases and inflammatory diseases (e.g., inflammatory bowel diseases (IBD), including ulcerative colitis and Crohn's disease) and cancer.

### SUMMARY

Existing strategies for modulating the immune system are flawed, in part because they are non-specific and can have undesirable side effects, are only targeted at individual cytokines or mechanisms, and are unable to be specifically localized to areas of inflammation. Provided herein is a technology that can be localized, dynamically controlled (e.g., based on timing or on sensing of an inflammatory state), and can target multiple arms of the immune system (e.g., adaptive immunity and innate immunity). In particular, the present disclosure provides engineered cell circuits that enable multifactorial modulation of immune systems.

Advantageously, these cell circuits may be engineered in eukaryotic cells, e.g., mesenchymal stem cells (MSCs), which are able to home to areas of inflammation, are able to produce an anti-inflammatory secretome, and are hypoimmunogenic, thus enabling their use for allogenic cell therapies, for example, without significant safety issues or side effects. These cell circuits, however, may also be engineered in other cell types, for example, cells of the immune system, such as T cells, B cells, natural killer (NK) cells, and dendritic cells (additional cell types are described herein).

As demonstrated herein, expressing combinations of certain effector molecules, such as IL-4 and IL-10, or IL-4 and IL-22, surprisingly results in a synergistic anti-inflammatory effect. These combinatorial anti-inflammatory cytokine-producing MSCs exhibit greater inhibitory capability than single anti-inflammatory cytokine MSCs in suppressing pro-inflammatory cytokine production by peripheral blood mononuclear cell (PBMC), for example (see, e.g., FIG. 14). Also surprising, this synergistic effect is observed even when low numbers/doses of engineered MSCs are used (see, e.g., FIG. 17).

Thus, some aspects of the present disclosure provide immune cells (e.g., mesenchymal stem cells (MSCs)) engineered to produce multiple effector molecules (e.g., two cytokines, or a cytokine and a homing molecule). In some embodiments, at least two of the effector molecules modulate different cell types of the immune system (e.g., one effector modulates one cell type, another effector modulates another cell type). In other embodiments, at least two of the effector molecules modulate the same cell type of the immune system (e.g., two effector molecules synergistically modulate the same cell type). In some embodiments, the MSCs comprise an engineered nucleic acid that comprises a promoter operably linked to a nucleotide sequence encoding an effector molecule. In some embodiments, the MSCs comprise an engineered nucleic acid that comprises a promoter operably linked to a nucleotide sequence encoding at least two effector molecules (e.g., as a fusion protein). In some embodiments, the MSCs comprise at least two engineered nucleic acids, each comprising a promoter operably linked to a nucleotide sequence encoding at least one (one or more) effector molecule.

In some embodiments, at least one effector molecule produced by the MSCs directly or indirectly modulates an innate immune cell and at least one effector molecule produced by the MSCs directly or indirectly modulates an adaptive immune cell.

In some embodiments, at least one effector molecule produced by the MSCs directly or indirectly modulates a pro-inflammatory cell and at least one effector molecule produced by the MSCs directly or indirectly modulates an anti-inflammatory cell.

In some embodiments, at least one effector molecule produced by the MSCs directly or indirectly modulates a myeloid cell and at least one effector molecule produced by the mesenchymal stem cell directly or indirectly modulates a lymphoid cell.

In some embodiments, the MSCs are engineered to produce a (one or more) homing molecule and/or a growth factor. In some embodiments, the MSCs are engineered to produce a homing molecule and an effector molecule (e.g., an anti-inflammatory cytokine). In some embodiments, the MSCs are engineered to produce two effector molecules, one of which is a homing molecule. In some embodiments, the mesenchymal stem cell is engineered to produce a homing molecule, in addition to anti-inflammatory effector molecule(s) or, optionally, in place of one or more (but not all) of the effector molecules, e.g., in place of one or more (but not all) of the anti-inflammatory cytokines.

Also provided herein, in some aspects, are methods that comprise culturing the engineered MSCs (under conditions suitable for gene expression) and producing the effector molecules.

Further provided herein, in some aspects, are methods that comprise delivering to a subject the engineered MSCs and producing (e.g., expressing) in vivo at least one effector molecule produced by the mesenchymal stem cell.

Further still, methods of treating a disease or disorder are provided. For example, methods may include treating an inflammatory bowel disease, such as ulcerative colitis or Crohn's disease, comprising delivering to the subject diagnosed with an inflammatory bowel disease engineered MSCs of the present disclosure (e.g., MSCs that express therapeutic effector molecules specifically for the treatment of inflammatory bowel disease).

The present disclosure also provide, in some aspects, methods of producing a multifunctional immunomodulatory cell, comprising (a) delivering to MSCs at least one engineered nucleic acid encoding at least two effector molecules, or (b) delivering to MSCs at least two engineered nucleic acids, each encoding at least one effector molecule, wherein each effector molecule modulates a different cell type of the immune system or modulates different functions of a cell.

Also provided herein are methods of modulating multiple cell types of the immune system of a subject, comprising delivering to the subject at least two MSCs, each engineered to produce an effector molecule, wherein at least two of the effector molecules modulate different cell types of the immune system.

In some embodiments, a (at least one) mesenchymal stem cell is engineered to produce two (at least two) anti-inflammatory cytokines at levels sufficient to inhibit an inflammatory response. The anti-inflammatory cytokines may be selected from IL-4, IL-10, and IL-22, for example. In some embodiments, the inflammatory response is inhibited by at least 20% (e.g., at least 30%, at least 40%, at least 50%, at least 60%, at least 70%, at least 80%, or at least 90%) relative to a control.

In some embodiments, the methods comprised delivering to a subject (e.g., an animal model, such as a mouse, or a human subject) a therapeutically effective amount of a preparation (e.g., a substantially pure preparation, e.g., containing less than 1% or less than 0.1% of other cell types) of mesenchymal stem cells engineered to produce two anti-inflammatory cytokines, wherein the therapeutically effective amount is sufficient to inhibit an inflammatory response in the subject. In some embodiments, the therapeutically effective amount is sufficient to inhibit the immune response by at least 20% (e.g., at least 30%, at least 40%, at least 50%, at least 60%, at least 70%, at least 80%, or at least 90%) relative to a control.

In some embodiments, a mesenchymal stem cell is derived from bone marrow, adipose tissue, or umbilical cord tissue. Other mesenchymal stem cell sources are contemplated herein.

In some embodiments, the anti-inflammatory cytokine levels are sufficient to induce a regulatory T cell immunophenotype (e.g., CD4+).

In some embodiments, the anti-inflammatory cytokine levels are sufficient to inhibit production of inflammatory cytokine by stimulated T cells by at least 20% (e.g., at least 30%, at least 40%, at least 50%, at least 60%, at least 70%, at least 80%, or at least 90%) relative to a control. In some embodiments, the control is an unmodified mesenchymal stem cell or a preparation of unmodified mesenchymal stem cells. In some embodiments, the inflammatory cytokines are selected from IFN-gamma, IL-17A, IL-1-beta, IL-6, and TNF-alpha. In some embodiments, the T cells are selected from CD8⁺ T cells, CD4⁺ T cells, gamma-delta T cells, and T regulatory cells.

In some embodiments, the mesenchymal stem cell is engineered to produce at least three anti-inflammatory cytokines at levels sufficient to inhibit an inflammatory response by at least 20% (e.g., at least 30%, at least 40%, at least 50%, at least 60%, at least 70%, at least 80%, or at least 90%) relative to a control.

In some embodiments, the mesenchymal stem cell is engineered to express a homing molecule. In some embodiments, the homing molecule is selected from: anti-integrin alpha4,beta7; anti-MAdCAM; CCR9; CXCR4; SDF1; MMP-2; CXCR1; CXCR7; CCR2; and GPR15. In some embodiments, the homing molecule is selected from: CXCR4, CCR2, CCR9, and GPR15.

In some embodiments, the mesenchymal stem cell comprises: (a) a nucleic acid comprising a promoter operably linked to a first nucleotide sequence encoding one of the two cytokines and a second nucleotide sequence encoding the other of the two cytokines, optionally wherein the first and second nucleotide sequence are separated by an intervening nucleotide sequence (e.g., an IRES element or a sequence encoding a 2A peptide, e.g., T2A, P2A, E2A, F2A (see, e.g., Ibrahimi et al. Hum Gene Ther. 2009 August; 20(8):845-60; and Kim et al. PLoS One. 2011; 6(4), incorporated herein by reference)); (b) a nucleic acid comprising (i) a first promoter operably linked to a nucleotide sequence encoding one of the two cytokines and (ii) a second promoter operably linked to a nucleotide sequence encoding the other of the two cytokines; or (c) a first nucleic acid comprising a first promoter operably linked to a nucleotide sequence encoding one of the two cytokines, and a second nucleic acid comprising a second promoter operably linked to a nucleotide sequence encoding the other of the two cytokines.

In some embodiments, the promoter of (a), the first and/or second promoter of (b), and/or the first and/or second promoter of (c) is an inducible promoter.

In some embodiments, the inducible promoter is a nuclear factor kappa-B (NF-κB)-responsive promoter. In some embodiments, the nucleic acid of (a), the nucleic acid of (b), and/or the first and/or second nucleic acid of (c) further comprises a promoter operably linked to a nucleotide sequence encoding a reporter molecule.

In some embodiments, a subject is symptomatic of having an inflammatory bowel disease (e.g., inflammation and/or sores (ulcers) in the innermost lining of the intestine (colon) and/or rectum). In some embodiments, a subject has been diagnosed with having an inflammatory bowel disease. In some embodiments, an inflammatory bowel disease is ulcerative colitis or Crohn's disease. The subject may be an animal or human subject.

In some embodiments, the therapeutically effective amount reduces weight loss in the subject by at least 20% (e.g., at least 30%, at least 40%, at least 50%, at least 60%, at least 70%, at least 80%, or at least 90%) relative to a control.

In some embodiments, the therapeutically effective amount reduces levels of lipocalin-2 in the subject by at least 20% (e.g., at least 30%, at least 40%, at least 50%, at least 60%, at least 70%, at least 80%, or at least 90%) relative to a control.

In some embodiments, the control is an unmodified mesenchymal stem cell or a preparation of unmodified mesenchymal stem cells.

Also provided herein are engineered nucleic acids comprising a promoter responsive to inflammatory cytokines operably linked to a nucleotide sequence encoding an effector molecule (e.g., an anti-inflammatory cytokine). In some embodiments, an engineered nucleic acid comprises a nuclear factor kappa-B (NF-κB)-responsive promoter operably linked to a nucleotide sequence encoding an effector molecule. In some embodiments, the effector molecule is an anti-inflammatory cytokine. For example, the anti-inflammatory cytokine may be selected from IL-4, IL-10, and IL-22.

### BRIEF DESCRIPTION OF THE DRAWINGS

FIG. 1 shows an example of a method for constructing an engineered nucleic acid that comprises a promoter operably linked to a nucleotide sequence encoding an effector molecule, cloned using a lentivirus plasmid backbone.

FIG. 2 shows an example of a method for testing engineered nucleic acids of the present disclosure in vitro to validate transgene function (left panel), in vivo to validate effector function (middle panel), and in a disease model (right panel) to validate efficacy.

FIGS. 3A-3B show efficacy data of nucleofection quantified by a pmaxGFP control. The microscopy image (FIG. 3A) was taken on a CYTELL™ device 21 hours after nucleofection. The flow cytometry data (FIG. 3B) was collected on a Sony Analyzer 24 hours after nucleofection. The histogram (FIG. 3B) shows the population of live mesenchymal stem cells (MSCs), gated based on size (forward scatter (FSC) vs. side scatter (SSC)) to match the size of the control, untransfected MSCs.

FIG. 4 shows a standard curve for interleukin-4 (IL-4) production by nucleofected MSCs. The standard curve for IL-4 was generated using the mixture of analyte standards included in the BD BIOLEGEND® kit, and the software package associated with the BD BIOLEGEND® kit.

FIG. 5 shows a histogram of IL-4 production by nucleofected MSCs. The histogram depicts the population of beads in the BD BIOLEGEND® kit that were labeled with anti-IL-4 antibody. These beads were isolated from all other beads using two nested gates: (1) FSC vs. SSC (size), and (2) allophycocyanin (APC) (fluorescence). In the BD BIOLEGEND® kit, the extent of IL-4 binding to the beads is correlated with phycoerythrin (PE) fluorescence, because the target cytokine is also bound by a secondary, PE-labeled antibody (similar to a sandwich enzyme-linked immunosorbent assay (ELISA)). This plot shows that MSCs that were nucleofected with DNA that encoded IL-4 production (the cytomegalovirus (CMV)-IL4 vector) produced enough IL-4 to saturate the standard curve, while all other conditions showed no change in secreted IL-4 relative to the untransfected control.

FIG. 6 shows a standard curve for interleukin-10 (IL-10) production by nucleofected MSCs. The standard curve for IL-10 was generated using the mixture of analyte standards included in the BD BIOLEGEND® kit, and the software package associated with the BD BIOLEGEND® kit.

FIG. 7 shows a histogram of IL-10 production by nucleofected MSCs. The histogram depicts the population of beads in the BD BIOLEGEND® kit that were labeled with anti-IL-10 antibody. These beads—isolated from all other beads using two nested gates: (1) FSC vs SSC (size), and (2) APC (fluorescence). In the BD BIOLEGEND® kit, the extent of IL-10 binding to the beads is correlated with PE fluorescence, because the target cytokine is also bound by a secondary, PE-labeled antibody (similar to a sandwich ELISA). This plot shows that MSCs that were nucleofected with DNA that encoded IL-10 production (the CMV-IL4 vector) produced enough IL-10 to saturate the standard curve, while all other conditions showed no change in secreted IL-10 relative to the untransfected control.

FIG. 8 is a graph showing the amount of interleulin-6 (IL-6) secreted by nucleofected MSCs. A BD BIOLEGEND® kit was used to determine the amount of IL-6 secreted by MSCs. This experiment evaluated whether electroporation alone or electroporation with a transgene encoding plasmid impacted IL-6 production. Quantification of the results was performed using a standard curve for IL-6 (generated using the BD BIOLEGEND® kit standards and software). This experiment showed that electroporation (using the LONZA® 4D AMAXA™, program # FF104) induced IL-6 secretion by LONZA® bone marrow-derived MSCs (BM-MSCs), and that this induction was further enhanced if transgene encoding DNA was included in the nucleofection reaction. The two replicates shown in FIG. 8 are technical replicates generated by the BD BIOLEGEND® analysis.

FIG. 9 shows schematics of stimulation conditions, induced cytokines, and engineered MSC effectors discussed in Example 2.

FIG. 10 shows schematics of the experimental design described in Example 2.

FIG. 11 shows graphs demonstrating that engineered MSCs express the appropriate anti-inflammatory cytokines. P=Stimulated peripheral blood mononuclear cells (PBMCs) only; P+M(cntl)=Stimulated PBMCs co-cultured with MSCs transfected with control plasmid; P+M(4)=Stimulated PBMCs co-cultured with MSCs transfected with IL-4 expression plasmid; P+M(10)=Stimulated PBMCs co-cultured with MSCs transfected with IL-10 expression plasmid; P+M(4/10)=Stimulated PBMCs co-cultured with MSCs transfected with IL-4 and IL-10 expression plasmids at half the amount of the single plasmids. Bars represent the mean of biological triplicates, error bars indicate standard error of the mean (S.E.M.).

FIG. 12 shows graphs demonstrating that engineered anti-inflammatory cytokine MSCs improve upon the intrinsic suppressive capabilities of MSCs on pro-inflammatory cytokine production by PBMCs.

FIG. 13 shows graphs demonstrating that engineered anti-inflammatory cytokine MSCs suppress pro-inflammatory cytokine production by PBMCs that control MSCs are unable to suppress on their own.

FIG. 14 shows graphs demonstrating that combination IL-4/IL-10 engineered anti-inflammatory cytokine MSCs demonstrate greater inhibitory capability than single engineered anti-inflammatory cytokine MSCs in suppressing pro-inflammatory cytokine production by PBMCs. Hash marks on mean bars indicate levels beyond upper limit of the graph's scale.

FIG. 15 shows a graph demonstrating that in some cases engineered anti-inflammatory cytokine MSCs did not confer any greater inhibitory capacity compared to control MSCs in suppressing pro-inflammatory cytokine production by PBMCs.

FIG. 16 shows a graph demonstrating that, in some cases, neither engineered anti-inflammatory cytokine MSCs nor control MSCs could suppress pro-inflammatory cytokine production by PBMCs.

FIG. 17 shows graphs demonstrating that engineered anti-inflammatory cytokine, even at diluted numbers, still demonstrate inhibition compared to diminished inhibitory capacity of diluted numbers of control MSCs in suppressing pro-inflammatory cytokine production by PBMCs.

FIG. 18 shows graphs demonstrating that engineered anti-inflammatory cytokine MSC (IL-4) induced additional anti-inflammatory cytokine production by PBMCs.

FIG. 19 shows a summary of cytokine production by ConA stimulated PBMCs, engineered MSCs, and co-cultured populations. NoTrans=MSCs not transfected; Trans-DNA=MSCs transfected without DNA; Trans+DNA=MCSs transfected with control plasmid; IL4 MSC=MSCs transfected with IL-4 expression plasmid; IL10 MSC=MSCs transfected with IL-10 expression plasmid; Combo DNA=MSCs transfected with IL-4 and IL-10 expression plasmids; Combo Cells=MSCs separately transfected with IL-4 or IL-10 expression plasmids, then mixed 1:1; aPBMCs=PBMCs stimulated with concanavalin A (ConA).

FIG. 20 shows a summary of cytokine production by ConA stimulated PBMCs, engineered MSCs, and co-cultured populations. NoTrans=MSCs not transfected; Trans-DNA=MSCs transfected without DNA; Trans+DNA=MCSs transfected with control plasmid; IL4 MSC=MSCs transfected with IL-4 expression plasmid; IL10 MSC=MSCs transfected with IL-10 expression plasmid; Combo DNA=MSCs transfected with IL-4 and IL-10 expression plasmids; Combo Cells=MSCs separately transfected with IL-4 or IL-10 expression plasmids, then mixed 1:1; aPBMCs=PBMCs stimulated with concanavalin A (ConA).

FIG. 21 shows that MSCs co-cultured with human CD4+ T cells can induce a regulatory T cell immunophenotype. Bar graphs show the percentage positive and MFI of the various culture conditions.

FIG. 22 shows that T cell stimulation-induced inflammatory cytokines are inhibited by MSCs engineered to secrete anti-inflammatory cytokine IL-4 or IL-10.

FIG. 23 shows that injected engineered MSCs expressing cytokines maintained cytokine expression in vivo. Each bar represents an average of 2-5 mice per group collected with error bars representing standard error of means (SEM).

FIG. 24 shows improved weight and survival from injected engineered MSCs in DSS colitis mice. Each cohort represents an average of 8 mice per group with error bars representing standard error of means (SEM).

FIG. 25 shows improved bloody stool and inflammatory lipocalin-2 levels from injected engineered MSCs in DSS colitis mice. Each cohort represents an average of 8 mice per group with error bars representing standard error of means (SEM).

FIG. 26 shows MSC biodistribution and persistence in DSS colitis mice. Fluorescence was measured as photons per seconds.

FIG. 27 shows MSC biodistribution and persistence within the colon and spleen in DSS colitis mice. Top-left is MSC-GFP, top-right is MSC-IL4, bottom-left is MSC-IL10, bottom-right is no MSC. Fluorescence was measured as photons per seconds.

FIG. 28 shows improved bloody stool and colon lengths from injected engineered MSCs specific to anti-inflammatory cytokines in DSS colitis mice. Injection cohorts and measurements were conducted in a double-blinded manner. Each cohort represents an average of 5 mice per group with error bars representing standard error of means (SEM).

FIGS. 29A and 29B show lentivirus workflow (FIG. 29A) and successful transduction of MSCs to generate engineered MSCs (FIG. 29B).

FIG. 30 shows lentiviral transduction to generate engineered MSCs resulted in desired cytokine expression absent inflammatory cytokine expression. Bars represent duplicate technical replicates.

FIG. 31 shows improved weight, colon length, lipocalin-2 levels, and colon histopathology and hyperplasia scoring from injected lentivirus engineered MSCs in DSS colitis mice. Each cohort represents an average of 8-10 mice per group with error bars representing standard error of means (SEM).

FIG. 32 shows improved weight, colon length, lipocalin-2 levels, and in situ colon inflammation L-012 levels from injected lentivirus engineered mouse IL-4/IL-22 combination MSCs in DSS colitis mice. Each cohort represents an average of 8-10 mice per group with error bars representing standard error of means (SEM).

FIG. 33 shows improved colon length and in situ colon inflammation L-012 levels from injected lentivirus engineered mouse IL-22 and IL-4/IL-22 combination MSCs in TNBS colitis mice. Each cohort represents an average of 5 mice per group with error bars representing standard error of means (SEM).

FIG. 34 shows secreted protein expression of mouse IL-22 as well as functional receptor signaling phospho-STAT3 activity of lentiviral transduced MSCs engineered to express mouse IL-22.

FIG. 35 shows the successful production, secretion, binding, and functional antagonism of TNF-alpha by a TNF-alpha Fab antibody certolizumab produced by engineered MSCs. All conditions were done as three biological replicates with error bars representing standard error of means (SEM).

FIG. 36 shows tissue biodistribution and increased homing of MSCs to inflamed colon by engineered expression of chemokine receptors CXCR4, CCR2, CCR9, and GPR15 in TNBS colitis mice. Luciferase chemiluminescence was measured as photons per seconds.

FIG. 37 shows a genetic circuit consisting of a conditional NF-kB (nuclear factor kappa-B) responsive promoter driving mouse IL-4 followed by a constitutive promoter driving GFP delivered by lentiviral transduction into MSCs enables them to sense inflammatory stimuli and respond via secretion of target payload IL-4. All conditions were done as three biological replicates with error bars representing standard error of means (SEM).

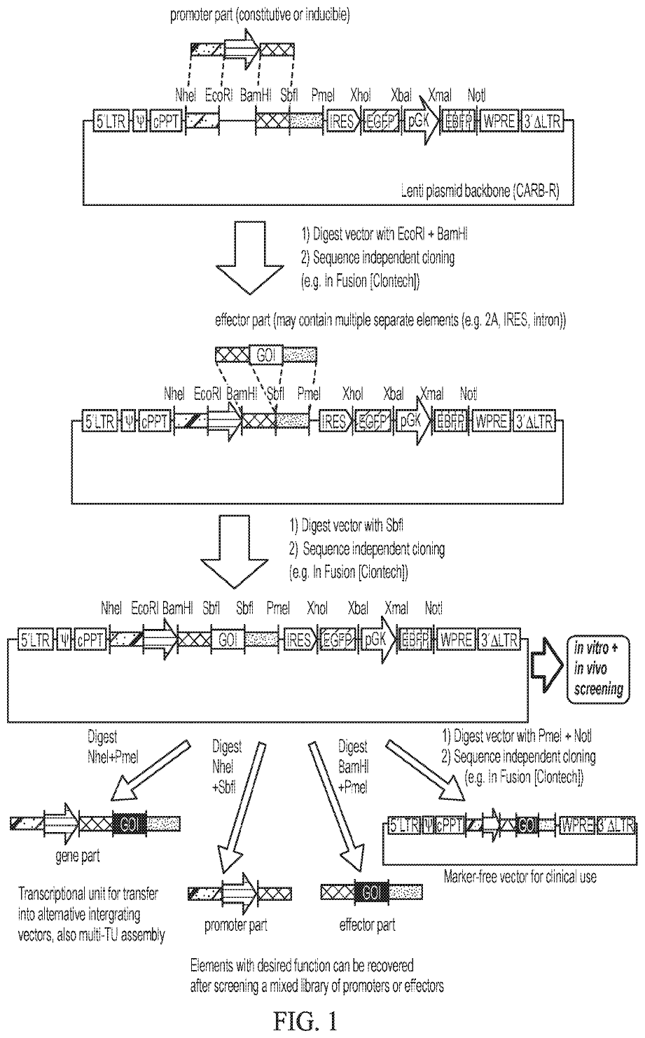

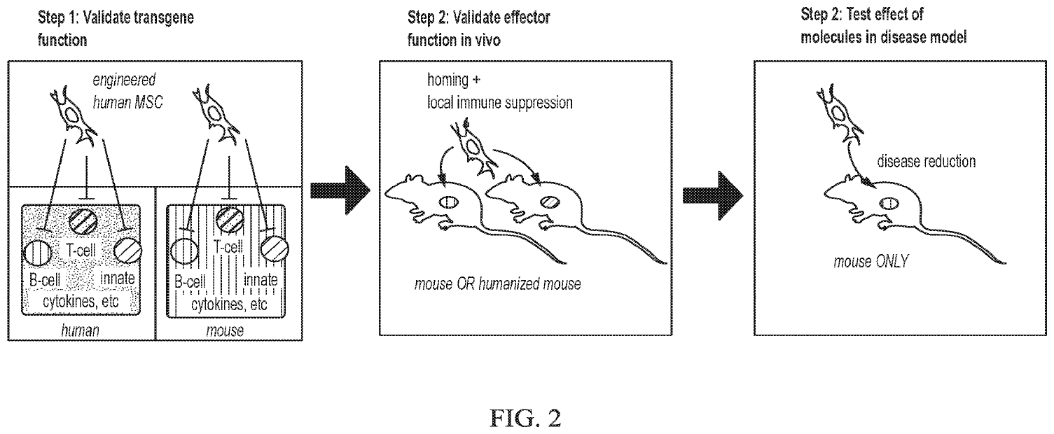

### DETAILED DESCRIPTION

Mesenchymal stem cells (MSCs) (also referred to as mesenchymal stromal cells) are a subset of non-hematopoietic adult stem cells that originate from the mesoderm. They possess self-renewal ability and multilineage differentiation into not only mesoderm lineages, such as chondrocytes, osteocytes and adipocytes, but also ectodermic cells and endodermic cells. MSCs, free of both ethical concerns and teratoma formation, are the major stem cell type used for cell therapy for treatment of both immune diseases and non-immune diseases. They can be easily isolated from the bone marrow, adipose tissue, the umbilical cord, fetal liver, muscle, and lung and can be successfully expanded in vitro. Further, MSCs have a tendency to home to damaged tissue sites. When MSCs are delivered exogenously and systemically administered to humans and animals, they migrate specifically to damaged tissue sites with inflammation. The inflammation-directed MSC homing involves several important cell trafficking-related molecules, including chemokines, adhesion molecules, and matrix metalloproteinases (MMPs).

Provided herein are methods of engineering MSCs (or other immune cell types) to produce effector molecules that modulate different cell types of the immune system or modulate different functions of a cell. These MSCs are referred to herein as “engineered MSCs.” These MSCs do not occur in nature. In some embodiments, the MSCs are engineered to include a nucleic acid (an engineered nucleic acid) comprising a promoter operably linked to a nucleotide sequence encoding an effector molecule. The promoter may be endogenous (e.g., genomically located in the cell) or exogenous (e.g., introduced into the cell as a component of the engineered nucleic acid).

It should be understood that the term “cell type” encompasses “cell subtypes.” Thus, an MSC that is engineered to produce both an effector molecule that targets a T cell and an effector molecule that targets a B cell is considered to target two different cell types. Likewise, an MSC that is engineered to produce both an effector molecule that targets a Th1 cell and an effector molecule that targets a Th17 cell (both subtypes of T cells) is also considered to target two different cell types.

An “effector molecule,” refers to a molecule (e.g., a nucleic acid such as DNA or RNA, or a protein (polypeptide) or peptide) that binds to another molecule and modulates the biological activity of that molecule to which it binds. For example, an effector molecule may act as a ligand to increase or decrease enzymatic activity, gene expression, or cell signaling. Thus, in some embodiments, an effector molecule modulates (activates or inhibits) a cell of the immune system. By directly binding to and modulating a molecule, an effector molecule may also indirectly modulate a second, downstream molecule. In some embodiments, an effector molecule is a secreted molecule, while in other embodiments, an effector molecule remains intracellular. For example, effector molecules include intracellular transcription factors, microRNA, and shRNAs that modify the internal cell state to, for example, enhance immunomodulatory activity, homing properties, or persistence of the cell. Non-limiting examples of effector molecules include cytokines, chemokines, enzymes that modulate metabolite levels, antibodies or decoy molecules that modulate cytokines, homing molecules, and/or integrins.

The term “modulate” encompasses maintenance of a biological activity, inhibition (partial or complete) of a biological activity, and activation (partial or complete) of a biological activity. The term also encompasses decreasing or increasing (e.g., enhancing) a biological activity. Two different effector molecules are considered to “modulate different cell types of the immune system” when one effector molecule modulates a type of cell (e.g., innate immune cell) that is different from the type of cell (e.g., adaptive immune cell) modulated by the other effector molecule.

Modulation by an effector molecule may be direct or indirect. Direct modulation occurs when an effector molecule binds to another molecule and modulates activity of that molecule. Indirect modulation occurs when an effector molecule binds to another molecule, modulates activity of that molecule, and as a result of that modulation, the activity of yet another molecule (to which the effector molecule is not bound) is modulated.

In some embodiments, modulation of a cell of the immune system results in an increase or a decrease in the biological activity of the cell by at least 10% (e.g., 10%, 20%, 30%, 40%, 50%, 60%, 70%, 80%, 90%, 100%, or 200%), relative to native biological activity of the cell. For example, modulation of a cell may result in an increase or a decrease in the biological activity of the cell by at least 20%, at least 30%, at least 40%, at least 50%, at least 60%, at least 70%, at least 80%, at least 90%, at least 100%, relative to native biological activity of the cell. In some embodiments, modulation of a cell of the immune system results in an increase or a decrease in the biological activity of the cell by 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%, relative to native biological activity of the cell.

In some embodiments, modulation of a cell of the immune system results in an increase or a decrease in the biological activity of the cell by at least 2 fold (e.g., 2, 3, 4, 5, 10, 25, 20, 25, 50, or 100 fold), relative to native biological activity of the cell. For example, modulation of a cell may result in an increase or a decrease in the biological activity of the cell by at least 3 fold, at least 5 fold, at least 10 fold, at least 20 fold, at least 50 fold, or at least 100 fold, relative to native biological activity of the cell. In some embodiments, modulating of a cell type of the immune system may lead to an increase or decrease of the number or activity of the cell in the immune system by 2-10, 2-20, 2-30, 2-40, 2-50, 2-60, 2-70, 2-80, 2-90, or 2-100 fold, relative to native biological activity of the cell.

“Native biological activity” of a cell refers to the biological activity of the cell in its natural environment, in the absence of an engineered MSC producing the effector molecule(s) (producing effector molecules not normally present in the environment of the cell in the immune system).

In some embodiments, MSCs are engineered to produce at least two (e.g., 2, 3, 4, 5, 6, 7, 8, 9, 10 or more) effector molecules, each of which modulates a different cell type of the immune system or modulates different functions of a cell. In other embodiments, MSCs are engineered to produce at least one effector molecule that is not natively produced by the MSCs. Such an effector molecule may, for example, complement the function of effector molecules natively produced by the MSCs.

In some embodiments, effector molecules function additively: the effect of two effector molecules, for example, is equal to the sum of the effect of the two effector molecules functioning separately. In other embodiments, effector molecules function synergistically: the effect of two effector molecules, for example, is greater than the combined function of the two effector molecules. The present disclosure also encompasses additivity and synergy between an effector molecule(s) and the immune cell from which they are produced.

Effector molecules that modulate cell types of the immune system may be, for example, secreted factors (e.g., cytokines, chemokines, antibodies, and/or decoy receptors that modulate extracellular mechanisms involved in the immune system), intracellular factors that control cell state (e.g., microRNAs and/or transcription factors that modulate the state of cells to enhance anti-inflammatory or pro-inflammatory properties), factors packaged into exosomes (e.g., microRNAs, cytosolic factors, and/or extracellular factors), surface displayed factors (e.g., checkpoint inhibitors), and and/or metabolic genes (e.g., enzymes that produce/modulate or degrade metabolites or amino acids).

In some embodiments, effector molecules may be selected from the following non-limiting classes of molecules: cytokines (e.g., IL-10), cytokine fusion proteins (e.g., IL-233), anti-cytokine antibodies (e.g., secukinumab, COSENTYX®; certolizumab, CIMZIA®), soluble cytokine receptors (e.g., IL-1RA), membrane bound cytokine receptors (e.g., mIL-1RAII), cytokine binding domain fusion proteins (e.g., etanercept, ENBREL®), cytokine binding proteins (e.g., IK18BP), anti-cytokine receptor antibodies (e.g., tocilizumab, ACTEMRA®), immune inhibitory receptors (e.g., PD-L), anti-activating receptor antibodies, ligands of activating receptor fusion proteins (e.g., abatacept, ORENCIA®), enzymes for the production of immunomodulatory compounds (e.g., iNOS), pathogenic effectors that suppress inflammation, antibodies against cell type-specific epitopes, chemokines, chemokine receptors, and transcription factors (e.g., transcription factors for induction or maintenance of MSC immunosuppressant state).

In some embodiments, MSCs comprise an engineered nucleic acid that comprises a promoter operably linked to a nucleotide sequence encoding an effector molecule. In some embodiments, an engineered nucleic acid comprises a promoter operably linked to a nucleotide sequence encoding at least 2 effector molecules. For example, the engineered nucleic acid may comprise a promoter operably linked to a nucleotide sequence encoding at least 3, at least 4, at least 5, at least 6, at least 7, at least 8, at least 9, or at least 10 effector molecules. In some embodiments, an engineered nucleic acid comprises a promoter operably linked to a nucleotide sequence encoding 1, 2, 3, 4, 5, 6, 7, 8, 9, 10, or more effector molecules.

MSCs, in some embodiments, are engineered to include at least two engineered nucleic acids, each comprising a promoter operably linked to a nucleotide sequence encoding at least one (e.g., 1, 2 or 3) effector molecule. For example, the MSCs may be engineered to comprise at least 2, at least 3, at least 4, at least 5, at least 6, at least 7, at least 8, at least 9, or at least 10, engineered nucleic acids, each comprising a promoter operably linked to a nucleotide sequence encoding at least one (e.g., 1, 2 or 3) effector molecule. In some embodiments, the MSCs are engineered to comprise 2, 3, 4, 5, 6, 7, 8, 9, 10, or more engineered nucleic acids, each comprising a promoter operably linked to a nucleotide sequence encoding at least one (e.g., 1, 2 or 3) effector molecule.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. The term “engineered nucleic acids” includes recombinant nucleic acids and synthetic nucleic acids. A “recombinant nucleic acid” refers to a molecule that is constructed by joining nucleic acid molecules and, in some embodiments, can replicate in a live cell. A “synthetic nucleic acid” refers to a molecule that is amplified or chemically, or by other means, synthesized. Synthetic nucleic acids include those that are chemically modified, or otherwise modified, but can base pair with naturally-occurring nucleic acid molecules. Recombinant nucleic acids and synthetic nucleic acids also include those molecules that result from the replication of either of the foregoing. Engineered nucleic acid of the present disclosure may be encoded by a single molecule (e.g., included in the same plasmid or other vector) or by multiple different molecules (e.g., multiple different independently-replicating molecules).

Engineered nucleic acid of the present disclosure may be produced using standard molecular biology methods (see, e.g., Green and Sambrook, Molecular Cloning, A Laboratory Manual, 2012, Cold Spring Harbor Press). In some embodiments, engineered nucleic acid constructs are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 'Y extension activity of a DNA polymerase and DNA ligase activity. The 5′ exonuclease activity chews back the 5′end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies. In some embodiments, engineered nucleic acid constructs are produced using IN-FUSION® cloning (Clontech).

A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. Nos. 4,683,202 and 5,928,906).

Promoters of an engineered nucleic acid may be “inducible promoters,” which refer to promoters that are characterized by regulating (e.g., initiating or activating) transcriptional activity when in the presence of, influenced by or contacted by a signal. The signal may be endogenous or a normally exogenous condition (e.g., light), compound (e.g., chemical or non-chemical compound) or protein (e.g., cytokine) that contacts an inducible promoter in such a way as to be active in regulating transcriptional activity from the inducible promoter. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

Non-limiting examples of promoters for use herein include promoter that are responsive to IFN-gamma, IL-17A, or TNF-alpha. A promoter is “responsive” to a signal if in the presence of that signal transcription from the promoter is activated, deactivated, increased or decreased. In some embodiments, the promoter comprises a response element. A “response element” is a short sequence of DNA within a promoter region that binds specific molecules (e.g., transcription factors) that modulate (regulate) gene expression from the promoter. Response elements that may be used in accordance with the present disclosure include, without limitation, an interferon-gamma-activated sequence (GAS) (Decker, T. et al. J Interferon Cytokine Res. 1997 March; 17(3):121-34, incorporated herein by reference), an interferon-stimulated response element (ISRE) (Han, K. J. et al. J Biol Chem. 2004 Apr. 9; 279(15):15652-61, incorporated herein by reference), a NF-kappaB response element (Wang, V. et al. Cell Reports. 2012; 2(4): 824-839, incorporated herein by reference), and a STAT3 response element (Zhang, D. et al. J of Biol Chem. 1996; 271: 9503-9509, incorporated herein by reference). Other response elements are encompassed herein.

Other non-limiting examples of promoters include the cytomegalovirus (CMV) promoter, the elongation factor 1-alpha (EF1a) promoter, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbC) promoter.

In some embodiments, a promoter of the present disclosure is modulated by an immune cell. An immune cell is considered to modulate a promoter if, in the presence of the immune cell (e.g., an immune cell that produces a molecule that increases or decreases activity of the promoter), the activity of the promoter is increased or decreased by at least 10%, relative to activity of the promoter in the absence of the immune cell. In some embodiments, the activity of the promoter is increased or decreased by at least 20%, at least 30%, at least 40%, at least 50%, at least 60%, at least 70%, at least 80%, at least 90%, at least 100%, relative to activity of the promoter in the absence of the immune cell. For example, the activity of the promoter is increased or decreased by 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%, relative to activity of the promoter in the absence of the immune cell.

In some embodiments, the activity of the promoter is increased or decreased by at least 2 fold (e.g., 2, 3, 4, 5, 10, 25, 20, 25, 50, or 100 fold), relative to activity of the promoter in the absence of the immune cell. For example, the activity of the promoter is increased or decreased by at least 3 fold, at least 5 fold, at least 10 fold, at least 20 fold, at least 50 fold, or at least 100 fold, relative to activity of the promoter in the absence of the immune cell. In some embodiments, the activity of the promoter is increased or decreased by 2-10, 2-20, 2-30, 2-40, 2-50, 2-60, 2-70, 2-80, 2-90, or 2-100 fold, relative to activity of the promoter in the absence of the immune cell.

In some embodiments, a promoter of the present disclosure is modulated by an immune cell selected from T cells, Th1 cells, Th17 cells, and M1 macrophage cells that secrete IFN-gamma, IL-17A, or TNF-alpha.

In some embodiments, a promoter of the present disclosure is activated under a hypoxic condition. A “hypoxic condition” is a condition where the body or a region of the body is deprived of adequate oxygen supply at the tissue level. Hypoxic conditions can cause inflammation (e.g., the level of inflammatory cytokines increase under hypoxic conditions). In some embodiments, the promoter that is activated under hypoxic condition is operably linked to a nucleotide encoding an effector molecule that decreases the expression of activity of inflammatory cytokines, thus reducing the inflammation caused by the hypoxic condition. In some embodiments, the promoter that is activated under hypoxic conditions comprises a hypoxia responsive element (HRE). A “hypoxia responsive element (HRE)” is a response element that responds to hypoxia-inducible factor (HIF). The HRE, in some embodiments, comprises a consensus motif NCGTG (where N is either A or G).

In some embodiments, engineered MSCs produce multiple effector molecules. For example, MSCs may be engineered to produce 2-20 different effector molecules. In some embodiments, MSCs engineered to produce 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 effector molecules. In some embodiments, MSCs are engineered to produce 1, 2, 3, 4, 5, 6, 7, 8, 9, 10, 11, 12, 13, 14, 15, 16, 17, 18, 19, or 20 effector molecules.

Engineered MSCs of the present disclosure produce multiple effector molecules, at least two of which modulate different cell types of the immune system.

In some embodiments, at least one effector molecule produced by an MSC directly or indirectly modulates an innate immune cell, and at least one effector molecule produced by the MSC directly or indirectly modulates an adaptive immune cell.

Innate immunity refers to nonspecific defense mechanisms that come into play immediately or within hours of an antigen's appearance in the body. These mechanisms include physical barriers such as skin, chemicals in the blood, and immune system cells that attack foreign cells in the body. The innate immune response is activated by chemical properties of the antigen. Examples of cells of the innate immune system include natural killer (NK) cells, NKT cells, mast cells, eosinophils, basophils, macrophages, neutrophils, and dendritic cells.

Adaptive immunity refers to antigen-specific immune response. The adaptive immune response is more complex than the innate immune response. The antigen first must be processed and recognized. Once an antigen has been recognized, the adaptive immune system creates an army of immune cells specifically designed to attack that antigen. Adaptive immunity also includes a “memory” that makes future responses against a specific antigen more efficient. Examples of cells of the adaptive immune system include T cells (e.g., from CD8⁺ T cells, CD4⁺ T cells, gamma-delta T cells, and T regulatory cells) and B cells.

In some embodiments, at least one effector molecule produced by an MSC directly or indirectly modulates a pro-inflammatory cell, and at least one effector molecule produced by the MSC directly or indirectly modulates an anti-inflammatory cell. Non-limiting examples of pro-inflammatory cells include M1 macrophages, M1 mesenchymal stem cells, effector T cells, Th17 cells, mature dendritic cells, and B cells. Non-limiting examples of anti-inflammatory cells include M2 macrophages, M2 mesenchymal stem cells, T regulatory cells, tolerogenic dendritic cells, regulatory B cells, and Tr1 cells.

In some embodiments, at least one effector molecule produced by an MSC directly or indirectly modulates a myeloid cell, and at least one effector molecule produced by the MSC directly or indirectly modulates a lymphoid cell. Non-limiting examples of myeloid cells include monocytes, macrophages, neutrophils, basophils, eosinophils, erythrocytes, dendritic cells, and megakaryocytes. Non-limiting examples of lymphoid cells include NK cells, T cells, and B cells.

In some embodiments, MSCs are engineered to produce at least one homing molecule. “Homing,” refers to active navigation (migration) of a cell to a target site (e.g., cell, tissue or organ). A “homing molecule” refers to a molecule that directs MSCs to a target site. In some embodiments, a homing molecule functions to recognize and/or initiate interaction of a MSC to a target site. Non-limiting examples of homing molecules include anti-integrin alpha4,beta7; anti-MAdCAM; CCR9; CXCR4; SDF1; MMP-2; CXCR1; and CXCR7.

In some embodiments, a homing molecule is a ligand that binds to selectin (e.g., hematopoietic cell E-/L-selectin ligand (HCELL), Dykstra et al., Stem Cells. 2016 October; 34(10):2501-2511) on the endothelium of a target tissue, for example.

In some embodiments, a homing molecule is a chemokine receptor (cell surface molecule that binds to a chemokine). Chemokines are small cytokines or signaling proteins secreted by cells that can induce directed chemotaxis in cells. Chemokines can be classified into four main subfamilies: CXC, CC, CX3C and XC, all of which exert biological effects by binding selectively to chemokine receptors located on the surface of target cells. Non-limiting examples of chemokine receptors that may be produced by the engineered MSCs of the present disclosure include: CXC chemokine receptors (e.g., CXCR1, CXCR2, CXCR3, CXCR4, CXCR5, CXCR6, and CXCR7), CC chemokine receptors (CCR1, CCR2, CCR3, CCR4, CCR5, CCR6, CCR7, CCR8, CCR9, CCR10, and CCR11), CX3C chemokine receptors (e.g., CX3C11), and XC chemokine receptors (e.g., XCR1). In some embodiments, a chemokine receptor is a G protein-linked transmembrane receptor. In some embodiments, MSCs are engineered to produce stromal cell-derived factor 1 (SDF1), also known as C—X—C motif chemokine 12 (CXCL12).

In some embodiments, a homing molecule is an integrin. Integrins are transmembrane receptors that facilitate cell-extracellular matrix (ECM) adhesion. Integrins are obligate heterodimers having two subunits: α (alpha) and β (beta). The a subunit of an integrin may be, without limitation: CD49a, CD49b, CD49c, CD49d, CD49e, CD49f, IGTA7, ITGA8, ITGA9, IGTA10, IGTA11, CD11D, CD103, CD11a, CD11b, CD51, CD41, and CD11c. The β subunit of an integrin may be, without limitation: CD29, CD18, CD61, CD104, ITGB5, ITGB6, ITGB7, and ITGB8. MSCs of the present disclosure may be engineered to produce any combination of the integrin α and β subunits.

In some embodiments, a homing molecule is a matrix metalloproteinase (MMP). MMPs are enzymes that cleave components of the basement membrane underlying the endothelial cell wall. Non-limiting examples of MMPs include MMP-2, MMP-9, and MMP. In some embodiments, MSCs are engineered to produce an inhibitor of a molecule (e.g., protein) that inhibits MMPs. For example, MSCs may be engineered to express an inhibitor (e.g., an RNAi molecule) of membrane type 1 MMP (MT1-MMP) or TIMP metallopeptidase inhibitor 1 (TIMP-1).

The term “homing molecule” also encompasses transcription factors that regulate the production of molecules that improve/enhance homing of MSCs.

In some embodiments, MSCs are engineered to produce at least one growth factor. A “growth factor” is a substance that stimulates cell growth, proliferation, differentiation and/or healing. Non-limiting examples of growth factors include platelet-derived growth factors (PDGFs), fibroblast growth factors (FGFs), epidermal growth factors (EGFs), and bone morphogenetic proteins (BMPs).

Other non-limiting examples of growth factors include: adrenomedullin (AM), angiopoietin (Ang), autocrine motility factor, bone morphogenetic proteins (BMPs), ciliary neurotrophic factor family, ciliary neurotrophic factor (CNTF), leukemia inhibitory factor (LIF), colony-stimulating factors, macrophage colony-stimulating factor (m-CSF), granulocyte colony-stimulating factor (G-CSF), granulocyte macrophage colony-stimulating factor (GM-CSF), ephrins, ephrin A1, ephrin A2, ephrin A3, ephrin A4, ephrin A5, ephrin B1, ephrin B2, ephrin B3, erythropoietin (EPO), fetal bovine somatotrophin (FBS), GDNF family of ligands, glial cell line-derived neurotrophic factor (GDNF), neurturin, persephin, artemin, growth differentiation factor-9 (GDF9), hepatocyte growth factor (HGF), hepatoma-derived growth factor (HDGF), insulin, insulin-like growth factors, insulin-like growth factor-1 (IGF-1), insulin-like growth factor-2 (IGF-2), interleukins, IL-1 (cofactor for IL-3 and IL-6, activates T cells), IL-2 (T-cell growth factor, stimulates IL-1 synthesis, activates B-cells and NK cells), IL-3 (stimulates production of all non-lymphoid cells), IL-4 (growth factor for activated B cells, resting T cells, and mast cells), IL-5 (induces differentiation of activated B cells and eosinophils), IL-6 (stimulates Ig synthesis, growth factor for plasma cells), IL-7 (growth factor for pre-B cells), keratinocyte growth factor (KGF), migration-stimulating factor (MSF), macrophage-stimulating protein (MSP), also known as hepatocyte growth factor-like protein (HGFLP), myostatin (GDF-8), neuregulins, neuregulin 1 (NRG1), neuregulin 2 (NRG2), neuregulin 3 (NRG3), neuregulin 4 (NRG4), neurotrophins, brain-derived neurotrophic factor (BDNF), nerve growth factor (NGF), neurotrophin-3 (NT-3), neurotrophin-4 (NT-4), placental growth factor (PGF), renalase (RNLS, anti-apoptotic survival factor), T-cell growth factor (TCGF), thrombopoietin (TPO), transforming growth factors, transforming growth factor alpha (TGF-α), transforming growth factor beta (TGF-β), tumor necrosis factor-alpha (TNF-α), vascular endothelial growth factor (VEGF), proteins in wnt signaling pathway, and growth factors in platelets.

In some embodiments, MSCs are engineered to produce at least one effector molecule that decreases expression or activity of an inflammatory cytokine. An “inflammatory cytokine” (also referred to as a “pro-inflammatory cytokine”) is a signaling molecule secreted from immune cells and certain other cell types that promotes inflammation. Non-limiting examples of inflammatory cytokine include interleukin-1 (IL-1), interferon gamma (IFN-gamma), IL-17A, IL-6, IL-1b, IL-8, IL-12(p70), IL-18, IL-23, tumor necrosis factor (TNF), and granulocyte-macrophage colony stimulating factor. Non-limiting examples of cells that produce inflammatory cytokines include T cells, Th1 cells, Th17 cells, and M1 macrophage cells, such as those that secrete IFN-gamma, IL-17A, or TNF-alpha.

An effector molecule is considered to decrease expression or activity of an inflammatory cytokine if the expression or activity of the inflammatory cytokine is decreased (reduced) by at least 10% (e.g., 10%, 20%, 30%, 40%, 50%, 60%, 70%, 80%, 90%, 100%, or 200%), relative to the native expression or activity of the inflammatory cytokine. “Native expression” of an inflammatory cytokine refers to the gene or protein expression level of the inflammatory cytokine in its natural environment, in the absence of an engineered MSC producing the effector molecule(s). “Native activity” of an inflammatory cytokine refers to the protein activity level of the inflammatory cytokine in its natural environment, in the absence of an engineered MSC that produces the effector molecule(s). Non-limiting examples of effector molecules that decrease expression or activity of an inflammatory cytokine include PD-L1 (B7H1), IL-1RA, soluble IFNR, ustekinumab, certolizumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, and CCL22 (see, e.g., Table 1).

In some embodiments, an effector molecule decreases expression or activity of an inflammatory cytokine by at least 20%, at least 30%, at least 40%, at least 50%, at least 60%, at least 70%, at least 80%, at least 90%, at least 100%, relative to the native expression or activity of the inflammatory cytokine. For example, an effector molecule may decrease expression or activity of an inflammatory cytokine by 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%, relative to the native expression or activity of the inflammatory cytokine.

In some embodiments, an effector molecule decreases expression or activity of an inflammatory cytokine by at least 2 fold (e.g., 2, 3, 4, 5, 10, 25, 20, 25, 50, or 100 fold), relative to the native expression or activity of the inflammatory cytokine. For example, an effector molecule may decrease expression or activity of an inflammatory cytokine by at least 3 fold, at least 5 fold, at least 10 fold, at least 20 fold, at least 50 fold, or at least 100 fold, relative to the native expression or activity of the inflammatory cytokine. In some embodiments, an effector molecule decreases expression or activity of an inflammatory cytokine by 2-10, 2-20, 2-30, 2-40, 2-50, 2-60, 2-70, 2-80, 2-90, or 2-100 fold, relative to the native expression or activity of the inflammatory cytokine.

In some embodiments, MSCs are engineered to produce at least one effector molecule that decreases expression or activity of an anti-inflammatory cytokine. An “anti-inflammatory cytokine” is a signaling molecule secreted from immune cells and certain other cell types that control the pro-inflammatory cytokine response. Non-limiting examples of anti-inflammatory cytokine include interleukin-4 (IL-4), IL-5, IL-10, IL-13, CCL2 and IL-33.

An effector molecule is considered to increase expression or activity of an anti-inflammatory cytokine if the expression or activity of the anti-inflammatory cytokine is increased by at least 10% (e.g., 10%, 20%, 30%, 40%, 50%, 60%, 70%, 80%, 90%, 100%, or 200%), relative to the native expression or activity of the anti-inflammatory cytokine. “Native expression” of an anti-inflammatory cytokine refers to the gene or protein expression level of the anti-inflammatory cytokine in its natural environment, in the absence of an engineered MSC that produces the effector molecule(s). “Native activity” of an anti-inflammatory cytokine refers to the protein activity level of the anti-inflammatory cytokine in its natural environment, in the absence of an engineered MSC that produces the effector molecule(s).

In some embodiments, an effector molecule increases expression or activity of an anti-inflammatory cytokine by at least 20%, at least 30%, at least 40%, at least 50%, at least 60%, at least 70%, at least 80%, at least 90%, at least 100%, relative to the native expression or activity of the anti-inflammatory cytokine. For example, an effector molecule may increase expression or activity of an anti-inflammatory cytokine by 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%, relative to the native expression or activity of the anti-inflammatory cytokine.

In some embodiments, an effector molecule increases expression or activity of an anti-inflammatory cytokine by at least 2 fold (e.g., 2, 3, 4, 5, 10, 25, 20, 25, 50, or 100 fold), relative to the native expression or activity of the anti-inflammatory cytokine. For example, an effector molecule may increase expression or activity of an anti-inflammatory cytokine by at least 3 fold, at least 5 fold, at least 10 fold, at least 20 fold, at least 50 fold, or at least 100 fold, relative to the native expression or activity of the anti-inflammatory cytokine. In some embodiments, an effector molecule increases expression or activity of an anti-inflammatory cytokine by 2-10, 2-20, 2-30, 2-40, 2-50, 2-60, 2-70, 2-80, 2-90, or 2-100 fold, relative to the native expression or activity of the anti-inflammatory cytokine.

In some embodiments, MSCs are engineered to produce at least one effector molecule that promotes conversion of T regulatory cells, increases the prevalence of T regulatory cells, or increases recruitment of T regulatory cells (e.g., systemically or locally such as at a site of tissue injury or inflammation). In some embodiments, MSCs are engineered to produce at least one effector molecule that promotes stability of a T regulatory phenotype. An effector molecule is considered to “promote conversion of T regulatory cells, increase the prevalence of T regulatory cells, or increase recruitment of T regulatory cells” if the number of T regulatory cells (e.g., CD4⁺, FOXP3⁺, CD25+T regulatory cells) systemically or at a site of inflammation (e.g., a diseased or damaged tissue) is increased by at least 10%, relative to the native T regulatory cell state. The “native T regulatory cell state” refers to the number and type of T cells present in a system or at a site of inflammation in the absence of the effector molecule. Non-limiting examples of effector molecule that promotes conversion of T regulatory cells, increases the prevalence of T regulatory cells, or increases recruitment of T regulatory cells include TGF-β, tocilizumab (anti-IL6), indoleamine 2,3-dioxygenase (IDO), IL-35, PD-L1, IL-2, and IL-2 variants.

In some embodiments, an effector molecule increases the number of T regulatory cells (e.g., CD4⁺, FOXP3⁺, CD25+T regulatory cells) systemically or at a site of inflammation by at least 20%, at least 30%, at least 40%, at least 50%, at least 60%, at least 70%, at least 80%, at least 90%, at least 100%, relative to the native T regulatory cell state. For example, an effector molecule may increase the number of T regulatory cells (e.g., CD4⁺, FOXP3⁺, CD25⁺ T regulatory cells) systemically or at a site of inflammation by 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%, relative to the native T regulatory cell state.

In some embodiments, an effector molecule increases the number of T regulatory cells (e.g., CD4⁺, FOXP3⁺, CD25⁺ T regulatory cells) systemically or at a site of inflammation by at least 2 fold (e.g., 2, 3, 4, 5, 10, 25, 20, 25, 50, or 100 fold), relative to the native T regulatory cell state. For example, an effector molecule may increase the number of T regulatory cells (e.g., CD4⁺, FOXP3⁺, CD25⁺ T regulatory cells) systemically or at a site of inflammation by at least 3 fold, at least 5 fold, at least 10 fold, at least 20 fold, at least 50 fold, or at least 100 fold, relative to the native T regulatory cell state. In some embodiments, an effector molecule increases the number of T regulatory cells (e.g., CD4⁺, FOXP3⁺, CD25⁺ T regulatory cells) systemically or at a site of inflammation by 2-10, 2-20, 2-30, 2-40, 2-50, 2-60, 2-70, 2-80, 2-90, or 2-100 fold, relative to the native T regulatory cell state.

In some embodiments, MSCs are engineered to produce IL-4, IL-6, or IL-10. In some embodiments, MSCs are engineered to produce IL-4, IL-6, and IL-10. In some embodiments, MSCs are engineered to produce IL-4 and IL-6. In some embodiments, MSCs are engineered to produce IL-4 and IL-10. In some embodiments, MSCs are engineered to produce IL-6 and IL-10.

In some embodiments, MSCs are engineered to produce IL-4 and/or IL-10, wherein at least one nucleic acid encoding the IL-4 and/or IL-10 is operably linked to a promoter that is responsive to IFN-gamma, IL-17A, TNF-alpha, IL-18, IL-23, IL-5, IL-13 and/or IL-1-beta. In some embodiments, an MSC comprises an engineered nucleic acid encoding IL-4 and/or IL-10, wherein the engineered nucleic acid is operably linked to a promoter that is responsive to IFN-gamma. In some embodiments, an MSC comprises an engineered nucleic acid encoding IL-4 and/or IL-10, wherein the engineered nucleic acid is operably linked to a promoter that is responsive to IL-17A. In some embodiments, an MSC comprises an engineered nucleic acid encoding IL-4 and/or IL-10, wherein the engineered nucleic acid is operably linked to a promoter that is responsive to TNF-alpha. In some embodiments, an MSC comprises an engineered nucleic acid encoding IL-4 and/or IL-10, wherein the engineered nucleic acid is operably linked to a promoter that is responsive to IL-18. some embodiments, an MSC comprises an engineered nucleic acid encoding IL-4 and/or IL-10, wherein the engineered nucleic acid is operably linked to a promoter that is responsive to IL-23. some embodiments, an MSC comprises an engineered nucleic acid encoding IL-4 and/or IL-10, wherein the engineered nucleic acid is operably linked to a promoter that is responsive to IL-5. some embodiments, an MSC comprises an engineered nucleic acid encoding IL-4 and/or IL-10, wherein the engineered nucleic acid is operably linked to a promoter that is responsive to IL-13. some embodiments, an MSC comprises an engineered nucleic acid encoding IL-4 and/or IL-10, wherein the engineered nucleic acid is operably linked to a promoter that is responsive to IL-1-beta.

### Cell Types of the Immune System

The immune system includes the innate immune system and the adaptive system, each including different types of cells with specific functions. The innate immune system comprises the cells and mechanisms that defend the host from infection by other organisms. The innate immune system, providing immediate defense against infection, recognizes and responds to a pathogen in a non-specific manner and does not provide long-lasting immunity to the host. The major functions of the innate immune system (e.g., in a vertebrate such as a mammal) include: recruiting immune cells to sites of infection through the production of chemical factors, including specialized chemical mediators called cytokines; activating the complement cascade to identify bacteria, activate cells, and promote clearance of antibody complexes or dead cells; identifying and removing foreign substances present in organs, tissues, blood and lymph by specialized white blood cells; activating the adaptive immune system through a process known as antigen presentation; and acting as a physical and chemical barrier to infectious agents.

Components of the innate immune system include physical barriers (skin, gastrointestinal tract, respiratory tract), defense mechanisms (secretions, mucous, bile), and general immune responses (inflammation). Leukocytes (also called white blood cells) and phagocytic cells are the main cell types that function in innate immune system and response, which identify and eliminate pathogens that might cause infection.

Leukocytes are not tightly associated with a particular organ or tissue and function similarly to that of independent, single-cell organisms. Leukocytes are able to move freely and interact with and capture cellular debris, foreign particles, and invading microorganisms. Unlike many other cells in the body, most innate immune leukocytes cannot divide or reproduce on their own, but are the products of multipotent hematopoietic stem cells present in the bone marrow. Types of leukocytes include, without limitation: mast cells, basophils, eosinophils, natural kill cells (NK cells), innate lymphoid cells (ILCs), and gamma-delta T cells.

Mast cells are a type of innate immune cell that reside in connective tissue and in the mucous membranes. Mast cells are associated with wound healing and defense against pathogens, but are also often associated with allergy and anaphylaxis. When activated, mast cells rapidly release characteristic granules, rich in histamine and heparin, along with various hormonal mediators and chemokines, or chemotactic cytokines into the environment. Histamine dilates blood vessels, causing the characteristic signs of inflammation, and recruits neutrophils and macrophages.

Basophils and eosinophils are cells related to the neutrophil. When activated by a pathogen encounter, histamine-releasing basophils are important in the defense against parasites and play a role in allergic reactions, such as asthma. Upon activation, eosinophils secrete a range of highly toxic proteins and free radicals that are highly effective in killing parasites, but may also damage tissue during an allergic reaction. Activation and release of toxins by eosinophils are, therefore, tightly regulated to prevent any inappropriate tissue destruction.

Natural killer cells (NK cells) are components of the innate immune system that do not directly attack invading microbes. Rather, NK cells destroy compromised host cells, such as tumor cells or virus-infected cells, which have abnormally low levels of a cell-surface marker called MHC I (major histocompatibility complex)—a situation that can arise in viral infections of host cells. NK cells are so named because of the initial notion that they do not require activation in order to kill cells with low surface MHC I molecules.

Gamma-delta T cells exhibit characteristics that place them at the border between innate and adaptive immunity. In some instances, gamma-delta T cells may be considered a component of adaptive immunity in that they rearrange TCR genes to produce junctional diversity and develop a memory phenotype. The various subsets may also be considered part of the innate immune system where a restricted TCR or NK receptors may be used as a pattern recognition receptor. For example, large numbers of Vgamma9/Vdelta2 T cells respond rapidly to common molecules produced by microbes, and highly restricted intraepithelial Vdelta1 T cells will respond to stressed epithelial cells.

Phagocytes are innate immune cells that engulf, or ‘phagocytose’, pathogens or particles. To engulf a particle or pathogen, a phagocyte extends portions of its plasma membrane, wrapping the membrane around the particle until it is enveloped (the particle is now inside the cell). Once inside the cell, the invading pathogen is contained inside an endosome, which merges with a lysosome. The lysosome contains enzymes and acids that kill and digest the particle or organism. In general, phagocytes patrol the body searching for pathogens, but are also able to react to a group of highly specialized molecular signals produced by other cells, called cytokines. Types of phagocytes include, without limitation: macrophages, neutrophils, and dendritic cells.

Macrophages are large phagocytic cells, which are able to move outside of the vascular system by migrating across the walls of capillary vessels and entering the areas between cells in pursuit of invading pathogens. In tissues, organ-specific macrophages are differentiated from phagocytic cells present in the blood called monocytes. Macrophages are the most efficient phagocytes and can phagocytose substantial numbers of bacteria or other cells or microbes. The binding of bacterial molecules to receptors on the surface of a macrophage triggers it to engulf and destroy the bacteria through the generation of a “respiratory burst,” causing the release of reactive oxygen species. Pathogens also stimulate the macrophage to produce chemokines, which recruit other cells to the site of infection. Macrophages that encourage inflammation are called M1 macrophages, whereas those that decrease inflammation and encourage tissue repair are called M2 macrophages.

Neutrophils, along with two other cell types (eosinophils and basophils), are known as granulocytes due to the presence of granules in their cytoplasm, or as polymorphonuclear cells (PMNs) due to their distinctive lobed nuclei. Neutrophil granules contain a variety of toxic substances that kill or inhibit growth of bacteria and fungi. Similar to macrophages, neutrophils attack pathogens by activating a respiratory burst. The main products of the neutrophil respiratory burst are strong oxidizing agents including hydrogen peroxide, free oxygen radicals and hypochlorite. Neutrophils are abundant and are usually the first cells to arrive at the site of an infection.

Dendritic cells (DCs) are phagocytic cells present in tissues that are in contact with the external environment, mainly the skin (where they are often called Langerhans cells), and the inner mucosal lining of the nose, lungs, stomach, and intestines. They are named for their resemblance to neuronal dendrites, but dendritic cells are not connected to the nervous system. Dendritic cells are very important in the process of antigen presentation, and serve as a link between the innate and adaptive immune systems.

Innate lymphoid cells (ILCs) play an important role in protective immunity and the regulation of homeostasis and inflammation. ILCs are classified based on the cytokines they produce and the transcription factors regulating their development and function. Group I ILCs produce type 1 cytokines and include natural killer cells. Group 2 ILCs produce type 2 cytokines, and Group 3 ILCs produce cytokines IL-17A and IL-22. Natural killer cells destroy compromised host cells, such as tumor cells or virus-infected cells. They can recognize stressed cells in the absence of antibodies, allowing them to react quickly to compromised host cells.

A myeloid cell is a cell that functions in the innate immune system. A myeloid cell includes, without limitation, monocytes, macrophages, neutrophils, basophils, eosinophils, erythrocytes, dendritic cells, and megakaryocytes or platelets. Lymphoid cells include T cells, B cells, and natural killer cells.

The adaptive immune system produces an adaptive immune response. An adaptive immune response, in its general form, begins with the sensitization of helper (TH, CD4⁺) and cytotoxic (CD8⁺) T cell subsets through their interaction with antigen presenting cells (APC) that express major histocompatibility (MHC)-class I or class II molecules associated with antigenic fragments (specific amino acid sequences derived from the antigen which bind to MHC I and/or MHC II for presentation on the cell surface). The sensitized or primed CD4+ T cells produce lymphokines that participate in the activation of B cells as well as various T cell subsets. The sensitized CD8⁺ T cells increase in numbers in response to lymphokines and are capable of destroying any cells that express the specific antigenic fragments associated with matching MHC-encoded class I molecules. Thus, in the course of a cancerous tumor, CTL eradicate cells expressing cancer associated or cancer specific antigens, thereby limiting the progression of tumor spread and disease development.

A “B lymphocyte” or “B cell” is a type of white blood cell. B cells function in the humoral immunity component of the adaptive immune system by secreting antibodies. B cells have two major functions: they present antigens to T cells, and more importantly, they produce antibodies to neutralize infectious microbes. Antibodies coat the surface of a pathogen and serve three major roles: neutralization, opsonization, and complement activation.

Neutralization occurs when the pathogen, because it is covered in antibodies, is unable to bind and infect host cells. In opsonization, an antibody-bound pathogen serves as a red flag to alert immune cells like neutrophils and macrophages, to engulf and digest the pathogen. Complement is a process for directly destroying, or lysing, bacteria.

Antibodies are expressed in two ways. The B-cell receptor (BCR), which sits on the surface of a B cell, is actually an antibody. B cells also secrete antibodies to diffuse and bind to pathogens. This dual expression is important because the initial problem, for instance a bacterium, is recognized by a unique BCR and activates the B cell. The activated B cell responds by secreting antibodies, essentially the BCR but in soluble form. This ensures that the response is specific against the bacterium that started the whole process.

Every antibody is unique, but they fall under general categories: IgM, IgD, IgG, IgA, and IgE. (Ig is short for immunoglobulin, which is another word for antibody.) While they have overlapping roles, IgM generally is important for complement activation; IgD is involved in activating basophils; IgG is important for neutralization, opsonization, and complement activation; IgA is essential for neutralization in the gastrointestinal tract; and IgE is necessary for activating mast cells in parasitic and allergic responses.

Memory B cell activation begins with the detection and binding of their target antigen, which is shared by their parent B cell. Some memory B cells can be activated without T cell help, such as certain virus-specific memory B cells, but others need T cell help. Upon antigen binding, the memory B cell takes up the antigen through receptor-mediated endocytosis, degrades it, and presents it to T cells as peptide pieces in complex with MHC-II molecules on the cell membrane. Memory T helper (TH) cells, typically memory follicular T helper (TFH) cells, that were derived from T cells activated with the same antigen recognize and bind these MHC-II-peptide complexes through their TCR. Following TCR-MHC-II-peptide binding and the relay of other signals from the memory TFH cell, the memory B cell is activated and differentiates either into plasmablasts and plasma cells via an extrafollicular response or enter a germinal center reaction where they generate plasma cells and more memory B cells.

Regulatory B cells (Bregs) represent a small population of B cells which participates in immuno-modulations and in suppression of immune responses. These cells regulate the immune system by different mechanisms. The main mechanism is a production of anti-inflammatory cytokine interleukin 10 (IL-10). The regulatory effects of Bregs were described in various models of inflammation, autoimmune diseases, transplantation reactions and in anti-tumor immunity.

T cells have a variety of roles and are classified by subsets. T cells are divided into two broad categories: CD8+ T cells or CD4+ T cells, based on which protein is present on the cell's surface. T cells carry out multiple functions, including killing infected cells and activating or recruiting other immune cells.

CD8+ T cells also are called cytotoxic T cells or cytotoxic lymphocytes (CTLs). They are crucial for recognizing and removing virus-infected cells and cancer cells. CTLs have specialized compartments, or granules, containing cytotoxins that cause apoptosis (programmed cell death). Because of its potency, the release of granules is tightly regulated by the immune system.

The four major CD4⁺ T-cell subsets are Th1, Th2, Th17, and Treg, with “Th” referring to “T helper cell.” Th1 cells are critical for coordinating immune responses against intracellular microbes, especially bacteria. They produce and secrete molecules that alert and activate other immune cells, like bacteria-ingesting macrophages. Th2 cells are important for coordinating immune responses against extracellular pathogens, like helminths (parasitic worms), by alerting B cells, granulocytes, and mast cells. Th17 cells are named for their ability to produce interleukin 17 (IL-17), a signaling molecule that activates immune and non-immune cells. Th17 cells are important for recruiting neutrophils.

Regulatory T cells (Tregs) monitor and inhibit the activity of other T cells. They prevent adverse immune activation and maintain tolerance, or the prevention of immune responses against the body's own cells and antigens. Type 1 regulatory T (Tr1) cells are an inducible subset of regulatory T cells that play a pivotal role in promoting and maintaining tolerance. The main mechanisms by which Tr1 cells control immune responses are the secretion of high levels of IL-10, and the killing of myeloid cells through the release of Granzyme B.

Memory T cells are a subset of antigen-specific T cells that persist long-term after an initial T cell response. They quickly expand to large numbers of effector T cells upon re-exposure to their cognate antigen, thus providing the immune system with “memory” against past antigens. The cancer vaccine described herein provides the immune system with “memory” against the tumor specific antigen, thereby eliciting strong immune response against newly emerged cancer cells or metastasized cancer cells.

A lymphocyte or lymphoid cell is a white blood cell in a vertebrate's adaptive immune system. Lymphocytes include natural killer cells (NK cells) (which function in cell-mediated, cytotoxic innate immunity), T cells (for cell-mediated, cytotoxic adaptive immunity), and B cells (for humoral, antibody-driven adaptive immunity).

### Examples of Engineered Stem Cells

The present disclosure primarily refers to mesenchymal stem cells (MSCs engineered to produce multiple effector molecules. It should be understood, however, that the present disclosure is not limited to engineered MSCs, but rather is intended to encompass other cell types of the immune system. For example, an engineered cell (engineered to produce effector molecules), as provided herein, may be selected from natural killer (NK) cells, NKT cells, mast cells, eosinophils, basophils, macrophages, neutrophils, and dendritic cells, T cells (e.g., CD8+ T cells, CD4+ T cells, gamma-delta T cells, and T regulatory cells (CD4+, FOXP3+, CD25+)) and B cells. Thus, MSCs of the present disclosure, in any embodiments, may be substituted for one of the foregoing immune cell types.

In some embodiments, the cell is a MSC engineered to produce multiple effector molecules, at least two of which modulate different cell types of the immune system. For example, one effector molecule may directly or indirectly modulate an innate immune cell, and another effector molecule may directly or indirectly modulates an adaptive immune cell. Non-limiting examples of innate immune cells include natural killer (NK) cells, NKT cells, mast cells, eosinophils, basophils, macrophages, neutrophils, and dendritic cells. Non-limiting examples of adaptive immune cells include T cells (e.g., CD8+ T cells, CD4+ T cells, gamma-delta T cells, and T regulatory cells (CD4⁺, FOXP3⁺, CD25⁺)) and B cells.

In some embodiments, MSCs are engineered to produce an effector molecule that modulates a NK cell and an effector molecule that modulates a T cell. In some embodiments, MSCs are engineered to produce an effector molecule that modulates a NKT cell and an effector molecule that modulates a T cell. In some embodiments, MSCs are engineered to produce an effector molecule that modulates a mast cell and an effector molecule that modulates a T cell. In some embodiments, MSCs are engineered to produce an effector molecule that modulates an eosinophil cell and an effector molecule that modulates a T cell. In some embodiments, MSCs are engineered to produce an effector molecule that modulates a basophil cell and an effector molecule that modulates a T cell. In some embodiments, MSCs are engineered to produce an effector molecule that modulates a macrophage cell and an effector molecule that modulates a T cell. In some embodiments, MSCs are engineered to produce an effector molecule that modulates a neutrophil cell and an effector molecule that modulates a T cell. In some embodiments, MSCs are engineered to produce an effector molecule that modulates a dendritic cell and an effector molecule that modulates a T cell.

In some embodiments, MSCs are engineered to produce an effector molecule that modulates a NK cell and an effector molecule that modulates a B cell. In some embodiments, MSCs are engineered to produce an effector molecule that modulates a NKB cell and an effector molecule that modulates a B cell. In some embodiments, MSCs are engineered to produce an effector molecule that modulates a mast cell and an effector molecule that modulates a B cell. In some embodiments, MSCs are engineered to produce an effector molecule that modulates an eosinophil cell and an effector molecule that modulates a B cell. In some embodiments, MSCs are engineered to produce an effector molecule that modulates a basophil cell and an effector molecule that modulates a B cell. In some embodiments, MSCs are engineered to produce an effector molecule that modulates a macrophage cell and an effector molecule that modulates a B cell. In some embodiments, MSCs are engineered to produce an effector molecule that modulates a neutrophil cell and an effector molecule that modulates a B cell. In some embodiments, MSCs are engineered to produce an effector molecule that modulates a dendritic cell and an effector molecule that modulates a B cell.

As another example, one effector molecule may directly or indirectly modulate a pro-inflammatory cell, and another effector molecule may directly or indirectly an anti-inflammatory cell. Non-limiting examples of pro-inflammatory cells include M1 macrophages, M1 mesenchymal stem cells, effector T cells, Th1 cells, Th17 cells, mature dendritic cells and B cells. Non-limiting examples of anti-inflammatory cells include M2 macrophages, M2 mesenchymal stem cells, T regulatory cells, tolerogenic dendritic cells, regulatory B cells, Th2 cells and Tr1 cells.

In some embodiments, MSCs are engineered to produce an effector molecule that modulates a M1 macrophage and an effector molecule that modulates a M2 macrophage. In some embodiments, MSCs are engineered to produce an effector molecule that modulates a M1 mesenchymal stem cell and an effector molecule that modulates a M2 macrophage. In some embodiments, MSCs are engineered to produce an effector molecule that modulates an effector T cell and an effector molecule that modulates a M2 macrophage. In some embodiments, MSCs are engineered to produce an effector molecule that modulates a Th1 cell and an effector molecule that modulates a M2 macrophage. In some embodiments, MSCs are engineered to produce an effector molecule that modulates a Th17 cell and an effector molecule that modulates a M2 macrophage. In some embodiments, MSCs are engineered to produce an effector molecule that modulates a mature dendritic cell and an effector molecule that modulates a M2 macrophage. In some embodiments, MSCs are engineered to produce an effector molecule that modulates a B cell and an effector molecule that modulates a M2 macrophage.

In some embodiments, MSCs are engineered to produce an effector molecule that modulates a M1 macrophage and an effector molecule that modulates a M2 mesenchymal stem cell. In some embodiments, MSCs are engineered to produce an effector molecule that modulates a M1 mesenchymal stem cell and an effector molecule that modulates a M2 mesenchymal stem cell. In some embodiments, MSCs are engineered to produce an effector molecule that modulates an effector T cell and an effector molecule that modulates a M2 mesenchymal stem cell. In some embodiments, MSCs are engineered to produce an effector molecule that modulates a Th1 cell and an effector molecule that modulates a M2 mesenchymal stem cell. In some embodiments, MSCs are engineered to produce an effector molecule that modulates a Th17 cell and an effector molecule that modulates a M2 mesenchymal stem cell. In some embodiments, MSCs are engineered to produce an effector molecule that modulates a mature dendritic cell and an effector molecule that modulates a M2 mesenchymal stem cell. In some embodiments, MSCs are engineered to produce an effector molecule that modulates a B cell and an effector molecule that modulates a M2 mesenchymal stem cell.

In some embodiments, MSCs are engineered to produce an effector molecule that modulates a M1 macrophage and an effector molecule that modulates a T regulatory cell. In some embodiments, MSCs are engineered to produce an effector molecule that modulates a M1 mesenchymal stem cell and an effector molecule that modulates a T regulatory cell. In some embodiments, MSCs are engineered to produce an effector molecule that modulates an effector T cell and an effector molecule that modulates a T regulatory cell. In some embodiments, MSCs are engineered to produce an effector molecule that modulates a Th1 cell and an effector molecule that modulates a T regulatory cell. In some embodiments, MSCs are engineered to produce an effector molecule that modulates a Th17 cell and an effector molecule that modulates a T regulatory cell. In some embodiments, MSCs are engineered to produce an effector molecule that modulates a mature dendritic cell and an effector molecule that modulates a T regulatory cell. In some embodiments, MSCs are engineered to produce an effector molecule that modulates a B cell and an effector molecule that modulates a T regulatory cell.

In some embodiments, MSCs are engineered to produce an effector molecule that modulates a M1 macrophage and an effector molecule that modulates a tolerogenic dendritic cell. In some embodiments, MSCs are engineered to produce an effector molecule that modulates a M1 mesenchymal stem cell and an effector molecule that modulates a tolerogenic dendritic cell. In some embodiments, MSCs are engineered to produce an effector molecule that modulates an effector T cell and an effector molecule that modulates a tolerogenic dendritic cell. In some embodiments, MSCs are engineered to produce an effector molecule that modulates a Th1 cell and an effector molecule that modulates a tolerogenic dendritic cell. In some embodiments, MSCs are engineered to produce an effector molecule that modulates a Th17 cell and an effector molecule that modulates a tolerogenic dendritic cell. In some embodiments, MSCs are engineered to produce an effector molecule that modulates a mature dendritic cell and an effector molecule that modulates a tolerogenic dendritic cell. In some embodiments, MSCs are engineered to produce an effector molecule that modulates a B cell and an effector molecule that modulates a tolerogenic dendritic cell.

In some embodiments, MSCs are engineered to produce an effector molecule that modulates a M1 macrophage and an effector molecule that modulates a regulatory B cell. In some embodiments, MSCs are engineered to produce an effector molecule that modulates a M1 mesenchymal stem cell and an effector molecule that modulates a regulatory B cell. In some embodiments, MSCs are engineered to produce an effector molecule that modulates an effector T cell and an effector molecule that modulates a regulatory B cell. In some embodiments, MSCs are engineered to produce an effector molecule that modulates a Th1 cell and an effector molecule that modulates a regulatory B cell. In some embodiments, MSCs are engineered to produce an effector molecule that modulates a Th17 cell and an effector molecule that modulates a regulatory B cell. In some embodiments, MSCs are engineered to produce an effector molecule that modulates a mature dendritic cell and an effector molecule that modulates a regulatory B cell. In some embodiments, MSCs are engineered to produce an effector molecule that modulates a B cell and an effector molecule that modulates a regulatory B cell.

In some embodiments, MSCs are engineered to produce an effector molecule that modulates a M1 macrophage and an effector molecule that modulates a Th2 cell. In some embodiments, MSCs are engineered to produce an effector molecule that modulates a M1 mesenchymal stem cell and an effector molecule that modulates a Th2 cell. In some embodiments, MSCs are engineered to produce an effector molecule that modulates an effector T cell and an effector molecule that modulates a Th2 cell. In some embodiments, MSCs are engineered to produce an effector molecule that modulates a Th1 cell and an effector molecule that modulates a Th2 cell. In some embodiments, MSCs are engineered to produce an effector molecule that modulates a Th17 cell and an effector molecule that modulates a Th2 cell. In some embodiments, MSCs are engineered to produce an effector molecule that modulates a mature dendritic cell and an effector molecule that modulates a Th2 cell. In some embodiments, MSCs are engineered to produce an effector molecule that modulates a B cell and an effector molecule that modulates a Th2 cell.

In some embodiments, MSCs are engineered to produce an effector molecule that modulates a M1 macrophage and an effector molecule that modulates a Tr1 cell. In some embodiments, MSCs are engineered to produce an effector molecule that modulates a M1 mesenchymal stem cell and an effector molecule that modulates a Tr1 cell. In some embodiments, MSCs are engineered to produce an effector molecule that modulates an effector T cell and an effector molecule that modulates a Tr1 cell. In some embodiments, MSCs are engineered to produce an effector molecule that modulates a Th1 cell and an effector molecule that modulates a Tr1 cell. In some embodiments, MSCs are engineered to produce an effector molecule that modulates a Th17 cell and an effector molecule that modulates a Tr1 cell. In some embodiments, MSCs are engineered to produce an effector molecule that modulates a mature dendritic cell and an effector molecule that modulates a Tr1 cell. In some embodiments, MSCs are engineered to produce an effector molecule that modulates a B cell and an effector molecule that modulates a Tr1 cell.

As yet another example, one effector molecule may directly or indirectly modulate a myeloid cell, and another effector molecule may directly or indirectly a lymphoid cell. Non-limiting examples of myeloid cells include monocytes, macrophages, neutrophils, basophils, eosinophils, erythrocytes, dendritic cells and megakaryocytes. Non-limiting examples of lymphoid cells include NK cells, T cells, and B cells.

In some embodiments, MSCs are engineered to produce an effector molecule that modulates a monocyte and an effector molecule that modulates a NK cell. In some embodiments, MSCs are engineered to produce an effector molecule that modulates a macrophage and an effector molecule that modulates a NK cell. In some embodiments, MSCs are engineered to produce an effector molecule that modulates a neutrophil and an effector molecule that modulates a NK cell. In some embodiments, MSCs are engineered to produce an effector molecule that modulates a basophil and an effector molecule that modulates a NK cell. In some embodiments, MSCs are engineered to produce an effector molecule that modulates an eosinophil and an effector molecule that modulates a NK cell. In some embodiments, MSCs are engineered to produce an effector molecule that modulates an erythrocyte and an effector molecule that modulates a NK cell. In some embodiments, MSCs are engineered to produce an effector molecule that modulates a dendritic cell and an effector molecule that modulates a NK cell. In some embodiments, MSCs are engineered to produce an effector molecule that modulates a megakaryocyte and an effector molecule that modulates a NK cell.

In some embodiments, MSCs are engineered to produce an effector molecule that modulates a monocyte and an effector molecule that modulates a T cell. In some embodiments, MSCs are engineered to produce an effector molecule that modulates a macrophage and an effector molecule that modulates a T cell. In some embodiments, MSCs are engineered to produce an effector molecule that modulates a neutrophil and an effector molecule that modulates a T cell. In some embodiments, MSCs are engineered to produce an effector molecule that modulates a basophil and an effector molecule that modulates a T cell. In some embodiments, MSCs are engineered to produce an effector molecule that modulates an eosinophil and an effector molecule that modulates a T cell. In some embodiments, MSCs are engineered to produce an effector molecule that modulates an erythrocyte and an effector molecule that modulates a T cell. In some embodiments, MSCs are engineered to produce an effector molecule that modulates a dendritic cell and an effector molecule that modulates a T cell. In some embodiments, MSCs are engineered to produce an effector molecule that modulates a megakaryocyte and an effector molecule that modulates a T cell.

In some embodiments, MSCs are engineered to produce an effector molecule that modulates a monocyte and an effector molecule that modulates a B cell. In some embodiments, MSCs are engineered to produce an effector molecule that modulates a macrophage and an effector molecule that modulates a B cell. In some embodiments, MSCs are engineered to produce an effector molecule that modulates a neutrophil and an effector molecule that modulates a B cell. In some embodiments, MSCs are engineered to produce an effector molecule that modulates a basophil and an effector molecule that modulates a B cell. In some embodiments, MSCs are engineered to produce an effector molecule that modulates an eosinophil and an effector molecule that modulates a B cell. In some embodiments, MSCs are engineered to produce an effector molecule that modulates an erythrocyte and an effector molecule that modulates a B cell. In some embodiments, MSCs are engineered to produce an effector molecule that modulates a dendritic cell and an effector molecule that modulates a B cell. In some embodiments, MSCs are engineered to produce an effector molecule that modulates a megakaryocyte and an effector molecule that modulates a B cell.

In some embodiments, MSCs are engineered to produce multiple effector molecules, each targeting a different cell T cell. For example, MSCs may be engineered to produce at least one (e.g., at least 2, 3 or 4) effector molecule that modulates (e.g., inhibits) Th1 cells and Th17 cells. As another example, MSCs may be engineered to produce at least one (e.g., at least 2, 3 or 4) effector molecule that inhibits Th1 cells and/or Th17 cells and at least one effector molecule that promotes conversion of T regulatory cells, increases the prevalence of T regulatory cells, increases recruitment of T regulatory cells, or promotes stability of T regulatory cells.

In some embodiments, in addition to producing multiple effector molecules, a MSC may be engineered to produce a homing molecule, a growth factor, or both a homing molecule and a growth factor.

Non-limiting examples of homing molecules include anti-integrin alpha4,beta7; anti-MAdCAM; CCR9; CXCR4; SDF1; MMP-2; CXCR1; and CXCR7. Thus, in some embodiments, MSCs are engineered to produce anti-integrin alpha4,beta7; anti-MAdCAM; CCR9; CXCR4; SDF1; MMP-2; CXCR1; and CXCR7; or any combination of two or more of the foregoing homing molecules.

Non-limiting examples of growth factors include PDGF, FGF, EGF and BMP. Thus, in some embodiments, MSCs are engineered to produce PDGF, FGF, EGF and BMP, or any combination of two or more of the foregoing growth factors.

In some embodiments, MSCs are engineered to produce at least one (e.g., at least 2 or at least 3) homing molecule selected from alpha4,beta7; anti-MAdCAM; CCR9; CXCR4; SDF1; MMP-2; CXCR1; and CXCR7, and at least one (e.g., at least 2 or at least 3) growth factor selected from PDGF, FGF, EGF and BMP.

Mesenchymal stem cells of the present disclosure typically comprise an engineered nucleic acid that comprises a promoter operably linked to a nucleotide sequence encoding an effector molecule. Non-limiting examples of promoters include the cytomegalovirus (CMV) promoter, the elongation factor 1-alpha (EF1a) promoter, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, or the ubiquitin C (UbC) promoter. The present disclosure also encompasses other native or synthetic promoters.

Non-limiting examples of effector molecules (e.g., encoded by the engineered nucleic acid) include PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, and CCL22 (see Table 1).

In some embodiments, the promoter is CMV and the effector molecule is PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM or anti-MMP9.

In some embodiments, the promoter is CMV and the effector molecule is PD-L1 (B7H1). In some embodiments, the promoter is CMV and the effector molecule is IL-1RA. In some embodiments, the promoter is CMV and the effector molecule is soluble IFNR. In some embodiments, the promoter is CMV and the effector molecule is ustekinumab. In some embodiments, the promoter is CMV and the effector molecule is p75 of TNFR. In some embodiments, the promoter is CMV and the effector molecule is anti-TNFalpha Nanobody®. In some embodiments, the promoter is CMV and the effector molecule is adalimumab. In some embodiments, the promoter is CMV and the effector molecule is MEDI2070. In some embodiments, the promoter is CMV and the effector molecule is IL-10. In some embodiments, the promoter is CMV and the effector molecule is IL-11. In some embodiments, the promoter is CMV and the effector molecule is IL-13. In some embodiments, the promoter is CMV and the effector molecule is IL-4. In some embodiments, the promoter is CMV and the effector molecule is IL-35. In some embodiments, the promoter is CMV and the effector molecule is IL-22. In some embodiments, the promoter is CMV and the effector molecule is IDO. In some embodiments, the promoter is CMV and the effector molecule is iNOS. In some embodiments, the promoter is CMV and the effector molecule is COX2. In some embodiments, the promoter is CMV and the effector molecule is HO1. In some embodiments, the promoter is CMV and the effector molecule is TSG-6. In some embodiments, the promoter is CMV and the effector molecule is Galectin-9. In some embodiments, the promoter is CMV and the effector molecule is LIF. In some embodiments, the promoter is CMV and the effector molecule is HLA-G5. In some embodiments, the promoter is CMV and the effector molecule is HIF-2-alpha. In some embodiments, the promoter is CMV and the effector molecule is anti-TL1A monoclonal antibody. In some embodiments, the promoter is CMV and the effector molecule is anti-integrin alpha4,beta7. In some embodiments, the promoter is CMV and the effector molecule is anti-MAdCAM. In some embodiments, the promoter is CMV and the effector molecule is anti-MMP9.

In some embodiments, the promoter is EF1a and the effector molecule is PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM or anti-MMP9.

In some embodiments, the promoter is EF1a and the effector molecule is PD-L1 (B7H1). In some embodiments, the promoter is EF1a and the effector molecule is IL-1RA. In some embodiments, the promoter is EF1a and the effector molecule is soluble IFNR. In some embodiments, the promoter is EF1a and the effector molecule is ustekinumab. In some embodiments, the promoter is EF1a and the effector molecule is p75 of TNFR. In some embodiments, the promoter is EF1a and the effector molecule is anti-TNFalpha Nanobody®. In some embodiments, the promoter is EF1a and the effector molecule is adalimumab. In some embodiments, the promoter is EF1a and the effector molecule is MEDI2070. In some embodiments, the promoter is EF1a and the effector molecule is IL-10. In some embodiments, the promoter is EF1a and the effector molecule is IL-11. In some embodiments, the promoter is EF1a and the effector molecule is IL-13. In some embodiments, the promoter is EF1a and the effector molecule is IL-4. In some embodiments, the promoter is EF1a and the effector molecule is IL-35. In some embodiments, the promoter is EF1a and the effector molecule is IL-22. In some embodiments, the promoter is EF1a and the effector molecule is IDO. In some embodiments, the promoter is EF1a and the effector molecule is iNOS. In some embodiments, the promoter is EF1a and the effector molecule is COX2. In some embodiments, the promoter is EF1a and the effector molecule is HO1. In some embodiments, the promoter is EF1a and the effector molecule is TSG-6. In some embodiments, the promoter is EF1a and the effector molecule is Galectin-9. In some embodiments, the promoter is EF1a and the effector molecule is LIF. In some embodiments, the promoter is EF1a and the effector molecule is HLA-G5. In some embodiments, the promoter is EF1a and the effector molecule is HIF-2-alpha. In some embodiments, the promoter is EF1a and the effector molecule is anti-TL1A monoclonal antibody. In some embodiments, the promoter is EF1a and the effector molecule is anti-integrin alpha4,beta7. In some embodiments, the promoter is EF1a and the effector molecule is anti-MAdCAM. In some embodiments, the promoter is EF1a and the effector molecule is anti-MMP9.

In some embodiments, the promoter is EFS and the effector molecule is PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM or anti-MMP9.

In some embodiments, the promoter is EFS and the effector molecule is PD-L1 (B7H1). In some embodiments, the promoter is EFS and the effector molecule is IL-1RA. In some embodiments, the promoter is EFS and the effector molecule is soluble IFNR. In some embodiments, the promoter is EFS and the effector molecule is ustekinumab. In some embodiments, the promoter is EFS and the effector molecule is p75 of TNFR. In some embodiments, the promoter is EFS and the effector molecule is anti-TNFalpha Nanobody®. In some embodiments, the promoter is EFS and the effector molecule is adalimumab. In some embodiments, the promoter is EFS and the effector molecule is MEDI2070. In some embodiments, the promoter is EFS and the effector molecule is IL-10. In some embodiments, the promoter is EFS and the effector molecule is IL-11. In some embodiments, the promoter is EFS and the effector molecule is IL-13. In some embodiments, the promoter is EFS and the effector molecule is IL-4. In some embodiments, the promoter is EFS and the effector molecule is IL-35. In some embodiments, the promoter is EFS and the effector molecule is IL-22. In some embodiments, the promoter is EFS and the effector molecule is IDO. In some embodiments, the promoter is EFS and the effector molecule is iNOS. In some embodiments, the promoter is EFS and the effector molecule is COX2. In some embodiments, the promoter is EFS and the effector molecule is HO1. In some embodiments, the promoter is EFS and the effector molecule is TSG-6. In some embodiments, the promoter is EFS and the effector molecule is Galectin-9. In some embodiments, the promoter is EFS and the effector molecule is LIF. In some embodiments, the promoter is EFS and the effector molecule is HLA-G5. In some embodiments, the promoter is EFS and the effector molecule is HIF-2-alpha. In some embodiments, the promoter is EFS and the effector molecule is anti-TL1A monoclonal antibody. In some embodiments, the promoter is EFS and the effector molecule is anti-integrin alpha4,beta7. In some embodiments, the promoter is EFS and the effector molecule is anti-MAdCAM. In some embodiments, the promoter is EFS and the effector molecule is anti-MMP9.

In some embodiments, the promoter is MND and the effector molecule is PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM or anti-MMP9.

In some embodiments, the promoter is MND and the effector molecule is PD-L1 (B7H1). In some embodiments, the promoter is MND and the effector molecule is IL-1RA. In some embodiments, the promoter is MND and the effector molecule is soluble IFNR. In some embodiments, the promoter is MND and the effector molecule is ustekinumab. In some embodiments, the promoter is MND and the effector molecule is p75 of TNFR. In some embodiments, the promoter is MND and the effector molecule is anti-TNFalpha Nanobody®. In some embodiments, the promoter is MND and the effector molecule is adalimumab. In some embodiments, the promoter is MND and the effector molecule is MEDI2070. In some embodiments, the promoter is MND and the effector molecule is IL-10. In some embodiments, the promoter is MND and the effector molecule is IL-11. In some embodiments, the promoter is MND and the effector molecule is IL-13. In some embodiments, the promoter is MND and the effector molecule is IL-4. In some embodiments, the promoter is MND and the effector molecule is IL-35. In some embodiments, the promoter is MND and the effector molecule is IL-22. In some embodiments, the promoter is MND and the effector molecule is IDO. In some embodiments, the promoter is MND and the effector molecule is iNOS. In some embodiments, the promoter is MND and the effector molecule is COX2. In some embodiments, the promoter is MND and the effector molecule is HO1. In some embodiments, the promoter is MND and the effector molecule is TSG-6. In some embodiments, the promoter is MND and the effector molecule is Galectin-9. In some embodiments, the promoter is MND and the effector molecule is LIF. In some embodiments, the promoter is MND and the effector molecule is HLA-G5. In some embodiments, the promoter is MND and the effector molecule is HIF-2-alpha. In some embodiments, the promoter is MND and the effector molecule is anti-TL1A monoclonal antibody. In some embodiments, the promoter is MND and the effector molecule is anti-integrin alpha4,beta7. In some embodiments, the promoter is MND and the effector molecule is anti-MAdCAM. In some embodiments, the promoter is MND and the effector molecule is anti-MMP9.

In some embodiments, the promoter is PGK and the effector molecule is PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM or anti-MMP9.

In some embodiments, the promoter is PGK and the effector molecule is PD-L1 (B7H1). In some embodiments, the promoter is PGK and the effector molecule is IL-1RA. In some embodiments, the promoter is PGK and the effector molecule is soluble IFNR. In some embodiments, the promoter is PGK and the effector molecule is ustekinumab. In some embodiments, the promoter is PGK and the effector molecule is p75 of TNFR. In some embodiments, the promoter is PGK and the effector molecule is anti-TNFalpha Nanobody®. In some embodiments, the promoter is PGK and the effector molecule is adalimumab. In some embodiments, the promoter is PGK and the effector molecule is MEDI2070. In some embodiments, the promoter is PGK and the effector molecule is IL-10. In some embodiments, the promoter is PGK and the effector molecule is IL-11. In some embodiments, the promoter is PGK and the effector molecule is IL-13. In some embodiments, the promoter is PGK and the effector molecule is IL-4. In some embodiments, the promoter is PGK and the effector molecule is IL-35. In some embodiments, the promoter is PGK and the effector molecule is IL-22. In some embodiments, the promoter is PGK and the effector molecule is IDO. In some embodiments, the promoter is PGK and the effector molecule is iNOS. In some embodiments, the promoter is PGK and the effector molecule is COX2. In some embodiments, the promoter is PGK and the effector molecule is HO1. In some embodiments, the promoter is PGK and the effector molecule is TSG-6. In some embodiments, the promoter is PGK and the effector molecule is Galectin-9. In some embodiments, the promoter is PGK and the effector molecule is LIF. In some embodiments, the promoter is PGK and the effector molecule is HLA-G5. In some embodiments, the promoter is PGK and the effector molecule is HIF-2-alpha. In some embodiments, the promoter is PGK and the effector molecule is anti-TL1A monoclonal antibody. In some embodiments, the promoter is PGK and the effector molecule is anti-integrin alpha4,beta7. In some embodiments, the promoter is PGK and the effector molecule is anti-MAdCAM. In some embodiments, the promoter is PGK and the effector molecule is anti-MMP9.

In some embodiments, the promoter is SFFV and the effector molecule is PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM or anti-MMP9.

In some embodiments, the promoter is SFFV and the effector molecule is PD-L1 (B7H1). In some embodiments, the promoter is SFFV and the effector molecule is IL-1RA. In some embodiments, the promoter is SFFV and the effector molecule is soluble IFNR. In some embodiments, the promoter is SFFV and the effector molecule is ustekinumab. In some embodiments, the promoter is SFFV and the effector molecule is p75 of TNFR. In some embodiments, the promoter is SFFV and the effector molecule is anti-TNFalpha Nanobody®. In some embodiments, the promoter is SFFV and the effector molecule is adalimumab. In some embodiments, the promoter is SFFV and the effector molecule is MEDI2070. In some embodiments, the promoter is SFFV and the effector molecule is IL-10. In some embodiments, the promoter is SFFV and the effector molecule is IL-11. In some embodiments, the promoter is SFFV and the effector molecule is IL-13. In some embodiments, the promoter is SFFV and the effector molecule is IL-4. In some embodiments, the promoter is SFFV and the effector molecule is IL-35. In some embodiments, the promoter is SFFV and the effector molecule is IL-22. In some embodiments, the promoter is SFFV and the effector molecule is IDO. In some embodiments, the promoter is SFFV and the effector molecule is iNOS. In some embodiments, the promoter is SFFV and the effector molecule is COX2. In some embodiments, the promoter is SFFV and the effector molecule is HO1. In some embodiments, the promoter is SFFV and the effector molecule is TSG-6. In some embodiments, the promoter is SFFV and the effector molecule is Galectin-9. In some embodiments, the promoter is SFFV and the effector molecule is LIF. In some embodiments, the promoter is SFFV and the effector molecule is HLA-G5. In some embodiments, the promoter is SFFV and the effector molecule is HIF-2-alpha. In some embodiments, the promoter is SFFV and the effector molecule is anti-TL1A monoclonal antibody. In some embodiments, the promoter is SFFV and the effector molecule is anti-integrin alpha4,beta7. In some embodiments, the promoter is SFFV and the effector molecule is anti-MAdCAM. In some embodiments, the promoter is SFFV and the effector molecule is anti-MMP9.

In some embodiments, the promoter is SV40 and the effector molecule is PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM or anti-MMP9.

In some embodiments, the promoter is SV40 and the effector molecule is PD-L1 (B7H1). In some embodiments, the promoter is SV40 and the effector molecule is IL-1RA. In some embodiments, the promoter is SV40 and the effector molecule is soluble IFNR. In some embodiments, the promoter is SV40 and the effector molecule is ustekinumab. In some embodiments, the promoter is SV40 and the effector molecule is p75 of TNFR. In some embodiments, the promoter is SV40 and the effector molecule is anti-TNFalpha Nanobody®. In some embodiments, the promoter is SV40 and the effector molecule is adalimumab. In some embodiments, the promoter is SV40 and the effector molecule is MEDI2070. In some embodiments, the promoter is SV40 and the effector molecule is IL-10. In some embodiments, the promoter is SV40 and the effector molecule is IL-11. In some embodiments, the promoter is SV40 and the effector molecule is IL-13. In some embodiments, the promoter is SV40 and the effector molecule is IL-4. In some embodiments, the promoter is SV40 and the effector molecule is IL-35. In some embodiments, the promoter is SV40 and the effector molecule is IL-22. In some embodiments, the promoter is SV40 and the effector molecule is IDO. In some embodiments, the promoter is SV40 and the effector molecule is iNOS. In some embodiments, the promoter is SV40 and the effector molecule is COX2. In some embodiments, the promoter is SV40 and the effector molecule is HO1. In some embodiments, the promoter is SV40 and the effector molecule is TSG-6. In some embodiments, the promoter is SV40 and the effector molecule is Galectin-9. In some embodiments, the promoter is SV40 and the effector molecule is LIF. In some embodiments, the promoter is SV40 and the effector molecule is HLA-G5. In some embodiments, the promoter is SV40 and the effector molecule is HIF-2-alpha. In some embodiments, the promoter is SV40 and the effector molecule is anti-TL1A monoclonal antibody. In some embodiments, the promoter is SV40 and the effector molecule is anti-integrin alpha4,beta7. In some embodiments, the promoter is SV40 and the effector molecule is anti-MAdCAM. In some embodiments, the promoter is SV40 and the effector molecule is anti-MMP9.

In some embodiments, the promoter is UbC and the effector molecule is PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM or anti-MMP9.

In some embodiments, the promoter is UbC and the effector molecule is PD-L1 (B7H1). In some embodiments, the promoter is UbC and the effector molecule is IL-1RA. In some embodiments, the promoter is UbC and the effector molecule is soluble IFNR. In some embodiments, the promoter is UbC and the effector molecule is ustekinumab. In some embodiments, the promoter is UbC and the effector molecule is p75 of TNFR. In some embodiments, the promoter is UbC and the effector molecule is anti-TNFalpha Nanobody®. In some embodiments, the promoter is UbC and the effector molecule is adalimumab. In some embodiments, the promoter is UbC and the effector molecule is MEDI2070. In some embodiments, the promoter is UbC and the effector molecule is IL-10. In some embodiments, the promoter is UbC and the effector molecule is IL-11. In some embodiments, the promoter is UbC and the effector molecule is IL-13. In some embodiments, the promoter is UbC and the effector molecule is IL-4. In some embodiments, the promoter is UbC and the effector molecule is IL-35. In some embodiments, the promoter is UbC and the effector molecule is IL-22. In some embodiments, the promoter is UbC and the effector molecule is IDO. In some embodiments, the promoter is UbC and the effector molecule is iNOS. In some embodiments, the promoter is UbC and the effector molecule is COX2. In some embodiments, the promoter is UbC and the effector molecule is HO1. In some embodiments, the promoter is UbC and the effector molecule is TSG-6. In some embodiments, the promoter is UbC and the effector molecule is Galectin-9. In some embodiments, the promoter is UbC and the effector molecule is LIF. In some embodiments, the promoter is UbC and the effector molecule is HLA-G5. In some embodiments, the promoter is UbC and the effector molecule is HIF-2-alpha. In some embodiments, the promoter is UbC and the effector molecule is anti-TL1A monoclonal antibody. In some embodiments, the promoter is UbC and the effector molecule is anti-integrin alpha4,beta7. In some embodiments, the promoter is UbC and the effector molecule is anti-MAdCAM. In some embodiments, the promoter is UbC and the effector molecule is anti-MMP9.

In some embodiments, MSCs comprise an engineered nucleic acid operably linked to a promoter modulated by an immune cell and encoding an effector molecule that decreases expression of an inflammatory cytokine or activity of an inflammatory cytokine.

In some embodiments, the immune cell is a T cell, and the promoter is responsive to IFN-gamma. In some embodiments, the immune cell is a T cell, and the promoter is responsive to IL-17A. In some embodiments, the immune cell is a T cell, and the promoter is responsive to TNFα. In some embodiments, the immune cell is a T cell, and the promoter comprises an interferon-gamma-activated sequence (GAS). In some embodiments, the immune cell is a T cell, and the promoter comprises an interferon-stimulated response element (ISRE). In some embodiments, the immune cell is a T cell, and the promoter comprises a NF-kappaB response element. In any of the foregoing embodiments wherein the immune cell is a T cell and the promoter is responsive to IFN-gamma, IL-17A, or TNFα, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a T cell and the promoter is responsive to IFN-gamma, IL-17A, or TNFα, the inflammatory cytokine may be IFN-gamma, IL-17A, IL-6, IFN-alpha, TNFα, IL-1b, IL-8, IL-12(p70), IL-18 or IL-23. In any of the foregoing embodiments wherein the immune cell is a T cell and the promoter comprises a GAS, ISRE, or NF-kappaB response element, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a T cell and the promoter comprises a GAS, ISRE, or NF-kappaB response element the inflammatory cytokine may be IFN-gamma, IL-17A, IL-6, IFN-alpha, TNFα, IL-1b, IL-8, IL-12(p70), IL-18 or IL-23.

In some embodiments, the immune cell is a Th1 cell, and the promoter is responsive to IFN-gamma. In some embodiments, the immune cell is a Th1 cell, and the promoter is responsive to IL-17A. In some embodiments, the immune cell is a Th1 cell, and the promoter is responsive to TNFα. In some embodiments, the immune cell is a Th1 cell, and the promoter comprises an interferon-gamma-activated sequence (GAS). In some embodiments, the immune cell is a Th1 cell, and the promoter comprises an interferon-stimulated response element (ISRE). In some embodiments, the immune cell is a Th1 cell, and the promoter comprises a NF-kappaB response element. In any of the foregoing embodiments wherein the immune cell is a Th1 cell and the promoter is responsive to IFN-gamma, IL-17A, or TNFα, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a Th1 cell and the promoter is responsive to IFN-gamma, IL-17A, or TNFα, the inflammatory cytokine may be IFN-gamma, IL-17A, IL-6, IFN-alpha, TNFα, IL-1b, IL-8, IL-12(p70), IL-18 or IL-23. In any of the foregoing embodiments wherein the immune cell is a Th1 cell and the promoter comprises a GAS, ISRE, or NF-kappaB response element, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a Th1 cell and the promoter comprises a GAS, ISRE, or NF-kappaB response element, the inflammatory cytokine may be IFN-gamma, IL-17A, IL-6, IFN-alpha, TNF-alpha, IL-1b, IL-8, IL-12(p70), IL-18 or IL-23.

In some embodiments, the immune cell is a Th17 cell, and the promoter is responsive to IFN-gamma. In some embodiments, the immune cell is a Th17 cell, and the promoter is responsive to IL-17A. In some embodiments, the immune cell is a Th17 cell, and the promoter is responsive to TNF-alpha. In some embodiments, the immune cell is a Th17 cell, and the promoter comprises an interferon-gamma-activated sequence (GAS). In some embodiments, the immune cell is a Th17 cell, and the promoter comprises an interferon-stimulated response element (ISRE). In some embodiments, the immune cell is a Th17 cell, and the promoter comprises a NF-kappaB response element. In any of the foregoing embodiments wherein the immune cell is a Th17 cell and the promoter is responsive to IFN-gamma, IL-17A, or TNF-alpha, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a Th17 cell and the promoter is responsive to IFN-gamma, IL-17A, or TNF-alpha, the inflammatory cytokine may be IFN-gamma, IL-17A, IL-6, IFN-alpha, TNF-alpha, IL-1b, IL-8, IL-12(p70), IL-18 or IL-23. In any of the foregoing embodiments wherein the immune cell is a Th17 cell and the promoter comprises a GAS, ISRE, or NF-kappaB response element, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a Th17 cell and the promoter comprises a GAS, ISRE, or NF-kappaB response element, the inflammatory cytokine may be IFN-gamma, IL-17A, IL-6, IFN-alpha, TNF-alpha, IL-1b, IL-8, IL-12(p70), IL-18 or IL-23.

In some embodiments, the immune cell is a M1 macrophage, and the promoter is responsive to IFN-gamma. In some embodiments, the immune cell is a M1 macrophage, and the promoter is responsive to IL-17A. In some embodiments, the immune cell is a M1 macrophage, and the promoter is responsive to TNF-alpha. In some embodiments, the immune cell is a M1 macrophage, and the promoter comprises an interferon-gamma-activated sequence (GAS). In some embodiments, the immune cell is a M1 macrophage, and the promoter comprises an interferon-stimulated response element (ISRE). In some embodiments, the immune cell is a M1 macrophage, and the promoter comprises a NF-kappaB response element. In any of the foregoing embodiments wherein the immune cell is a M1 macrophage and the promoter is responsive to IFN-gamma, IL-17A, or TNF-alpha, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a M1 macrophage and the promoter is responsive to IFN-gamma, IL-17A, or TNF-alpha, the inflammatory cytokine may be IFN-gamma, IL-17A, IL-6, IFN-alpha, TNF-alpha, IL-1b, IL-8, IL-12(p70), IL-18 or IL-23. In any of the foregoing embodiments wherein the immune cell is a M1 macrophage and the promoter comprises a GAS, ISRE, or NF-kappaB response element, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a M1 macrophage and the promoter comprises a GAS, ISRE, or NF-kappaB response element, the inflammatory cytokine may be IFN-gamma, IL-17A, IL-6, IFN-alpha, TNF-alpha, IL-1b, IL-8, IL-12(p70), IL-18 or IL-23.

In some embodiments, MSCs comprise engineered nucleic acids operably linked to a promoter activated in the presence of IFNγ, IL-17A or TNF-alpha and encoding an effector molecule that decreases expression of an inflammatory cytokine or activity of an inflammatory cytokine. In some embodiments, the promoter comprises a response element selected from GAS, an ISRE, a NF-kappaB response element, and a STAT3 response element. In any of the foregoing embodiments wherein the promoter is activated in the presence of IFNγ, IL-17A or TNF-alpha, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the promoter is activated in the presence of IFNγ, IL-17A or TNF-alpha, the inflammatory cytokine may be IFN-gamma, IL-17A, IL-6, IFN-alpha, TNF-alpha, IL-1b, IL-8, IL-12(p70), IL-18 or IL-23.

In some embodiments, MSCs comprise engineered nucleic acids operably linked to a promoter activated under hypoxic conditions and encoding an effector molecule that decreases expression of an inflammatory cytokine or activity of an inflammatory cytokine. The promoter may comprise, for example, a hypoxia responsive element (HRE). In some embodiments, the promoter is responsive to HIF-1a transcription factor. In some embodiments, the effector molecule is PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In some embodiments, the inflammatory cytokine may be IFN-gamma, IL-17A, IL-6, IFN-alpha, TNF-alpha, IL-1b, IL-8, IL-12(p70), IL-18 or IL-23.

In some embodiments, MSCs comprise engineered nucleic acids operably linked to a promoter modulated by an immune cell and encoding an effector molecule that decreases expression of an anti-inflammatory cytokine or activity of an anti-inflammatory cytokine.

In some embodiments, the immune cell is a T cell, and the promoter is responsive to IFN-gamma. In some embodiments, the immune cell is a T cell, and the promoter is responsive to IL-17A. In some embodiments, the immune cell is a T cell, and the promoter is responsive to TNF-alpha. In some embodiments, the immune cell is a T cell, and the promoter comprises an interferon-gamma-activated sequence (GAS). In some embodiments, the immune cell is a T cell, and the promoter comprises an interferon-stimulated response element (ISRE). In some embodiments, the immune cell is a T cell, and the promoter comprises a NF-kappaB response element. In any of the foregoing embodiments wherein the immune cell is a T cell and the promoter is responsive to IFN-gamma, IL-17A, or TNF-alpha, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a T cell and the promoter is responsive to IFN-gamma, IL-17A, or TNF-alpha, the anti-inflammatory cytokine may be IL-4, IL-5, IL-10, IL-13, CCL2 or IL-33. In any of the foregoing embodiments wherein the immune cell is a T cell and the promoter comprises a GAS, ISRE, or NF-kappaB response element, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a T cell and the promoter comprises a GAS, ISRE, or NF-kappaB response element, the anti-inflammatory cytokine may be IL-4, IL-5, IL-10, IL-13, CCL2 or IL-33.

In some embodiments, the immune cell is a Th1 cell, and the promoter is responsive to IFN-gamma. In some embodiments, the immune cell is a Th1 cell, and the promoter is responsive to IL-17A. In some embodiments, the immune cell is a Th1 cell, and the promoter is responsive to TNF-alpha. In some embodiments, the immune cell is a Th1 cell, and the promoter comprises an interferon-gamma-activated sequence (GAS). In some embodiments, the immune cell is a Th1 cell, and the promoter comprises an interferon-stimulated response element (ISRE). In some embodiments, the immune cell is a Th1 cell, and the promoter comprises a NF-kappaB response element. In any of the foregoing embodiments wherein the immune cell is a Th1 cell and the promoter is responsive to IFN-gamma, IL-17A, or TNF-alpha, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a Th1 cell and the promoter is responsive to IFN-gamma, IL-17A, or TNF-alpha, the anti-inflammatory cytokine may be IL-4, IL-5, IL-10, IL-13, CCL2 or IL-33. In any of the foregoing embodiments wherein the immune cell is a Th1 cell and the promoter comprises a GAS, ISRE, or NF-kappaB response element, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a Th1 cell and the promoter comprises a GAS, ISRE, or NF-kappaB response element the anti-inflammatory cytokine may be IL-4, IL-5, IL-10, IL-13, CCL2 or IL-33.

In some embodiments, the immune cell is a Th17 cell, and the promoter is responsive to IFN-gamma. In some embodiments, the immune cell is a Th17 cell, and the promoter is responsive to IL-17A. In some embodiments, the immune cell is a Th17 cell, and the promoter is responsive to TNF-alpha. In some embodiments, the immune cell is a Th17 cell, and the promoter comprises an interferon-gamma-activated sequence (GAS). In some embodiments, the immune cell is a Th17 cell, and the promoter comprises an interferon-stimulated response element (ISRE). In some embodiments, the immune cell is a Th17 cell, and the promoter comprises a NF-kappaB response element. In any of the foregoing embodiments wherein the immune cell is a Th17 cell and the promoter is responsive to IFN-gamma, IL-17A, or TNF-alpha, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a Th17 cell and the promoter is responsive to IFN-gamma, IL-17A, or TNF-alpha, the anti-inflammatory cytokine may be IL-4, IL-5, IL-10, IL-13, CCL2 or IL-33. In any of the foregoing embodiments wherein the immune cell is a Th17 cell and the promoter comprises a GAS, ISRE, or NF-kappaB response element, IL-17A, or TNF-alpha, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a Th17 cell and the promoter comprises a GAS, ISRE, or NF-kappaB response element, the anti-inflammatory cytokine may be IL-4, IL-5, IL-10, IL-13, CCL2 or IL-33.

In some embodiments, the immune cell is a M1 macrophage, and the promoter is responsive to IFN-gamma. In some embodiments, the immune cell is a M1 macrophage, and the promoter is responsive to IL-17A. In some embodiments, the immune cell is a M1 macrophage, and the promoter is responsive to TNF-alpha. In some embodiments, the immune cell is a M1 macrophage, and the promoter comprises an interferon-gamma-activated sequence (GAS). In some embodiments, the immune cell is a M1 macrophage, and the promoter comprises an interferon-stimulated response element (ISRE). In some embodiments, the immune cell is a M1 macrophage, and the promoter comprises a NF-kappaB response element. In any of the foregoing embodiments wherein the immune cell is a M1 macrophage and the promoter is responsive to IFN-gamma, IL-17A, or TNF-alpha, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a M1 macrophage and the promoter is responsive to IFN-gamma, IL-17A, or TNF-alpha, the anti-inflammatory cytokine may be IL-4, IL-5, IL-10, IL-13, CCL2 or IL-33. In any of the foregoing embodiments wherein the immune cell is a M1 macrophage and the promoter comprises a GAS, ISRE, or NF-kappaB response element, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the immune cell is a M1 macrophage and the promoter comprises a GAS, ISRE, or NF-kappaB response element, the anti-inflammatory cytokine may be IL-4, IL-5, IL-10, IL-13, CCL2 or IL-33.

In some embodiments, MSCs comprise engineered nucleic acids operably linked to a promoter activated in the presence of IFNγ, IL-17A or TNF-alpha and encoding an effector molecule that decreases expression of an anti-inflammatory cytokine or activity of an anti-inflammatory cytokine. In some embodiments, the promoter comprises a response element selected from GAS, an ISRE, a NF-kappaB response element, and a STAT3 response element. In any of the foregoing embodiments wherein the promoter is activated in the presence of IFNγ, IL-17A or TNF-alpha, the effector molecule may be PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In any of the foregoing embodiments wherein the promoter is activated in the presence of IFNγ, IL-17A or TNF-alpha, the anti-inflammatory cytokine may be IL-4, IL-5, IL-10, IL-13, CCL2 or IL-33.

In some embodiments, MSCs comprise engineered nucleic acids operably linked to a promoter activated under hypoxic conditions and encoding an effector molecule that decreases expression of an anti-inflammatory cytokine or activity of an anti-inflammatory cytokine. The promoter may comprise, for example, a hypoxia responsive element (HRE). In some embodiments, the promoter is responsive to HIF-1a transcription factor. In some embodiments, the effector molecule is PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, or CCL22. In some embodiments, the anti-inflammatory cytokine may be IL-4, IL-5, IL-10, IL-13, CCL2 or IL-33.

In some embodiments, MSCs comprise engineered nucleic acids operably linked to a promoter modulated by an immune cell and encoding an effector molecule that promotes conversion of T regulatory cells, increases the prevalence of T regulatory cells, or increases recruitment of T regulatory cells.

In some embodiments, the immune cell is a T cell, and the promoter is responsive to IFN-gamma. In some embodiments, the immune cell is a T cell, and the promoter is responsive to IL-17A. In some embodiments, the immune cell is a T cell, and the promoter is responsive to TNF-alpha. In some embodiments, the immune cell is a T cell, and the promoter comprises an interferon-gamma-activated sequence (GAS). In some embodiments, the immune cell is a T cell, and the promoter comprises an interferon-stimulated response element (ISRE). In some embodiments, the immune cell is a T cell, and the promoter comprises a NF-kappaB response element. In any of the foregoing embodiments wherein the immune cell is a T cell and the promoter is responsive to IFN-gamma, IL-17A, or TNF-alpha, the effector molecule may be TGF-beta, tocilizumab (anti-IL6), indoleamine 2,3-dioxygenase (IDO), IL-35, PD-L1, IL-2 or an IL-2 variant. In any of the foregoing embodiments wherein the immune cell is a T cell and the promoter comprises a GAS, ISRE, or NF-kappaB response element, the effector molecule may be TGF-beta, tocilizumab (anti-IL6), indoleamine 2,3-dioxygenase (IDO), IL-35, PD-L1, IL-2 or an IL-2 variant.

In some embodiments, the immune cell is a Th1 cell, and the promoter is responsive to IFN-gamma. In some embodiments, the immune cell is a Th1 cell, and the promoter is responsive to IL-17A. In some embodiments, the immune cell is a Th1 cell, and the promoter is responsive to TNF-alpha. In some embodiments, the immune cell is a Th1 cell, and the promoter comprises an interferon-gamma-activated sequence (GAS). In some embodiments, the immune cell is a Th1 cell, and the promoter comprises an interferon-stimulated response element (ISRE). In some embodiments, the immune cell is a Th1 cell, and the promoter comprises a NF-kappaB response element. In any of the foregoing embodiments wherein the immune cell is a Th1 cell and the promoter is responsive to IFN-gamma, IL-17A, or TNF-alpha, the effector molecule may be TGF-beta, tocilizumab (anti-IL6), indoleamine 2,3-dioxygenase (IDO), IL-35, PD-L1, IL-2 or an IL-2 variant. In any of the foregoing embodiments wherein the immune cell is a Th1 cell and the promoter comprises a GAS, ISRE, or NF-kappaB response element, the effector molecule may be TGF-beta, tocilizumab (anti-IL6), indoleamine 2,3-dioxygenase (IDO), IL-35, PD-L1, IL-2 or an IL-2 variant.

In some embodiments, the immune cell is a Th17 cell, and the promoter is responsive to IFN-gamma. In some embodiments, the immune cell is a Th17 cell, and the promoter is responsive to IL-17A. In some embodiments, the immune cell is a Th17 cell, and the promoter is responsive to TNF-alpha. In some embodiments, the immune cell is a Th17 cell, and the promoter comprises an interferon-gamma-activated sequence (GAS). In some embodiments, the immune cell is a Th17 cell, and the promoter comprises an interferon-stimulated response element (ISRE). In some embodiments, the immune cell is a Th17 cell, and the promoter comprises a NF-kappaB response element. In any of the foregoing embodiments wherein the immune cell is a Th17 cell and the promoter is responsive to IFN-gamma, IL-17A, or TNF-alpha, the effector molecule may be TGF-beta, tocilizumab (anti-IL6), indoleamine 2,3-dioxygenase (IDO), IL-35, PD-L, IL-2 or an IL-2 variant. In any of the foregoing embodiments wherein the immune cell is a Th17 cell and the promoter comprises a GAS, ISRE, or NF-kappaB response element, the effector molecule may be TGF-beta, tocilizumab (anti-IL6), indoleamine 2,3-dioxygenase (IDO), IL-35, PD-L1, IL-2 or an IL-2 variant.

In some embodiments, the immune cell is a M1 macrophage, and the promoter is responsive to IFN-gamma. In some embodiments, the immune cell is a M1 macrophage, and the promoter is responsive to IL-17A. In some embodiments, the immune cell is a M1 macrophage, and the promoter is responsive to TNF-alpha. In some embodiments, the immune cell is a M1 macrophage, and the promoter comprises an interferon-gamma-activated sequence (GAS). In some embodiments, the immune cell is a M1 macrophage, and the promoter comprises an interferon-stimulated response element (ISRE). In some embodiments, the immune cell is a M1 macrophage, and the promoter comprises a NF-kappaB response element. In any of the foregoing embodiments wherein the immune cell is a M1 macrophage and the promoter is responsive to IFN-gamma, IL-17A, or TNF-alpha, the effector molecule may be TGF-beta, tocilizumab (anti-IL6), indoleamine 2,3-dioxygenase (IDO), IL-35, PD-L1, IL-2 or an IL-2 variant. In any of the foregoing embodiments wherein the immune cell is a M1 macrophage and the promoter comprises a GAS, ISRE, or NF-kappaB response element, the effector molecule may be TGF-beta, tocilizumab (anti-IL6), indoleamine 2,3-dioxygenase (IDO), IL-35, PD-L1, IL-2 or an IL-2 variant.

In some embodiments, MSCs comprise engineered nucleic acids operably linked to a promoter activated in the presence of IFNγ, IL-17A or TNF-alpha and encoding an effector molecule that promotes conversion of T regulatory cells, increases the prevalence of T regulatory cells, or increases recruitment of T regulatory cells. In some embodiments, the promoter comprises a response element selected from GAS, an ISRE, a NF-kappaB response element, and a STAT3 response element. In any of the foregoing embodiments wherein the promoter is activated in the presence of IFNγ, IL-17A or TNF-alpha, the effector molecule may be TGF-beta, tocilizumab (anti-IL6), indoleamine 2,3-dioxygenase (IDO), IL-35, PD-L1, IL-2 or an IL-2 variant.

In some embodiments, MSCs comprise engineered nucleic acids operably linked to a promoter activated under hypoxic conditions and encoding an effector molecule that that promotes conversion of T regulatory cells, increases the prevalence of T regulatory cells. The promoter may comprise, for example, a hypoxia responsive element (HRE). In some embodiments, the promoter is responsive to HIF-1a transcription factor. In some embodiments, the effector molecule is TGF-beta, tocilizumab (anti-IL6), indoleamine 2,3-dioxygenase (IDO), IL-35, PD-L1, IL-2 or an IL-2 variant.

### Methods

Also provided herein are methods that include culturing the engineered MSCs of the present disclosure. Methods of culturing MSCs are known. In some embodiments, MSCs are culture in growth medium (e.g., MSCGM human Mesenchymal Stem Cell Growth BULLETKIT™ Medium (serum containing), THERAPEAK™ MSCGM-CD™ Mesenchymal Stem Cell Chemically Defined Medium (serum free), or RoosterBio xeno-free MSC media).

Further provided herein are methods that include delivering, or administering, to a subject (e.g., a human subject) engineered MSCs as provided herein to produce in vivo at least one effector molecule produced by the MSCs. In some embodiments, the MSCs are administered intravenously, intraperitoneally, systemically or locally (e.g., to a site of inflammation). In some embodiments, the MSCs are administered prior to the peak if inflammation or at the peak of inflammation.

Some methods comprise selecting a subject (or patient population) having a specific inflammatory marker that is dysregulated and treating that subject with engineered MSCs that modulate the dysregulated inflammatory marker. For example, subject may have elevated TNF alpha and may be treated with engineered MSCs that produce effector molecules (e.g., anti-TNF alpha molecules), the expression of which is regulated by a TNF alpha-responsive promoter.

The engineered MSCs of the present disclosure may be used, in some instances, to treat inflammatory bowel disease, such as ulcerative colitis or Crohn's disease. Other autoimmune and/or inflammatory disorders are encompassed herein. For example, engineered MSCs may be delivered to subjects having Alzheimer's disease, ankylosing spondylitis, arthritis (e.g., osteoarthritis, rheumatoid arthritis (RA), psoriatic arthritis), asthma, atherosclerosis, dermatitis, diverticulitis, fibromyalgia, hepatitis, systemic lupus erythematous (SLE), nephritis, or Parkinson's disease.

TABLE 1
Example Effector Molecules

| Protein Name | Description/Function | Reference |
|---|---|---|
| Programmed death- | immune inhibitory receptor ligand; inhibits | J. Immunol. 170 |
| ligand 1 (PD-L1)/B7 | T-cell activation and cytokine production | (3), 1257-1266 |
| homolog 1 (B7-H1) | | (2003) |
| Interleukin-1 receptor | inhibits the activities of interleukin 1, alpha | Proc. Natl. Acad. |
| antagonist (IL-1RA) | (IL1A) and interleukin 1, beta (IL1B); | Sci. U.S.A. 88 (9), |
| | modulates a variety of interleukin 1 related | 3681-3685 (1991) |
| | immune and inflammatory responses | |
| Interferon production | functions in the induction of class II MHC | Proc Natl Acad Sci |
| regulator (IFNR) | antigens by IFN-gamma | USA. 1991 Jul |
| | | 15; 88(14): 6077-81 |
| Ustekinumab | human monoclonal antibody, directed | Therapeutics and |
| (STELARA ®) | against interleukin 12 and interleukin 23 to | clinical risk |
| | activate certain T cells | management. 6, |
| | | 123-41 (2010) |
| p75 TNFR/TNFR2 | encoded by this gene is a member of the | Int |
| | TNF-receptor superfamily; forms | Immunopharmacol. |
| | heterocomplex with TNF-receptor 1 to | 2015 |
| | mediate the recruitment of two anti- | Sep; 28(1): 146-53 |
| | apoptotic proteins, c-IAP1 and c-IAP2, | |
| | which possess E3 ubiquitin ligase activity | |
| anti-TNFalpha | Nanobodies ™ against Tumor Necrosis | US20100297111 |
| Nanobody ® | Factor-alpha (TNF-alpha) | |
| certolizumab | A TNF-alpha Fab antibody sold under the | MAbs. |
| | trade name CIMZIA ®; used for the | 2010 |
| | treatment of Crohn's disease, rheumatoid | Mar-Apr; 2(2): 137-147 |
| | arthritis, psoriatic arthritis and ankylosing | |
| | spondylitis | |
| adalimumab | an inhibitory human monoclonal antibody | U.S. Pat. No. |
| | against tumor necrosis factor-alpha (TNF- | 6,090,382 |
| | alpha); sold under the trade name | |
| | HUMIRA ® among others; a medication | |
| | used to treat rheumatoid arthritis, psoriatic | |
| | arthritis, ankylosing spondylitis, Crohn's | |
| | disease, ulcerative colitis, chronic psoriasis, | |
| | hidradenitis suppurativa, and juvenile | |
| | idiopathic arthritis | |
| MEDI2070 | an IL-23 monoclonal antibody designed for | WO2014143540 |
| | the treatment of Crohn's disease | |
| Interleukin-10 (IL-10) | inhibits the synthesis of a number of | Structure. 2005 |
| | cytokines, including IFN-gamma, IL-2, IL- | Apr; 13(4): 551-64 |
| | 3, TNF and GM-CSF produced by a variety | |
| | of cell lines, including activated | |
| | macrophages, helper T-cells, mast cells and | |
| | other cell types | |
| Interleukin-11 (IL-11) | a cytokine that stimulates the proliferation | Proc Natl Acad Sci |
| | of hematopoietic stem cells and | USA. 1990 |
| | megakaryocyte progenitor cells; induces | Oct; 87(19): 7512-6 |
| | megakaryocyte maturation resulting in | |
| | increased platelet production, promotes the | |
| | proliferation of hepatocytes in response to | |
| | liver damage | |
| Interleukin-13 (IL-13) | a cytokine that inhibits inflammatory | Proc Natl Acad Sci |
| | cytokine production; critical in regulating | USA. 1993 Apr |
| | inflammatory and immune responses | 15; 90(8): 3735-9 |
| Interleukin-4 (IL-4) | participates in at least several B-cell | Proc Natl Acad Sci |
| | activation processes as well as of other cell | USA. 1986 |
| | types; induces the expression of class II | Aug; 83(16): 5894-8 |
| | MHC molecules on resting B-cells; | |
| | enhances both secretion and cell surface | |
| | expression of IgE and IgG1; regulates the | |
| | expression of the low affinity Fc receptor | |
| | for IgE (CD23) on both lymphocytes and | |
| | monocytes | |
| Interleukin-35 (IL-35) | regulates T cell and inflammatory, in part | PLoS ONE. 7 (3): |
| | by activating the Jak/STAT pathway of | e33628, 2012 |
| | CD4+ T cells | |
| Interleukin-22 (IL-22) | cytokine that contributes to the | The Journal of |
| | inflammatory response in vivo | Biological |
| | | Chemistry |
| | | 275, 31335-31339, |
| | | 2000 |
| Nitric oxide synthase, | a nitric oxide synthase which is expressed | Proc Natl Acad Sci |
| inducible (iNOS) | in liver and is inducible by a combination | USA. 90 (8): 3491-5, |
| | of lipopolysaccharide and certain cytokines | 1993 |
| Cyclooxygenase, | an enzyme that is encoded by the PTGS2 | Crit Rev Neurobiol. |
| isoform 2 (COX2) | gene; involved in the conversion of | 13 (1): 45-82 |
| | arachidonic acid to prostaglandin H2; an | |
| | important precursor of prostacyclin | |
| Heme oxygenase 1 | stress protein induced by oxidative stress, | Biochem. Biophys. |
| (HO1) | such as UVA radiation, hydrogen peroxide, | Res. Commun. 338 |
| | and sodium arsenite; catalyzes the | (1): 558-67 |
| | degradation of heme. | |
| TNF-stimulated gene 6 | a secretory protein that contains a | J Biol Chem. 2016 |
| protein (TSG-6) | hyaluronan-binding domain; induced by | Jun 10; 291(24): |
| | pro-inflammatory cytokines such as tumor | 12627-40 |
| | necrosis factor alpha and interleukin-1; | |
| | enhances the serine protease inhibitory | |
| | activity of I alpha I, which is important in | |
| | the protease network associated with | |
| | inflammation | |
| Galectin-9 | belongs to a family of proteins defined by | Glycobiology. 12 |
| | their binding specificity for β-galactoside | (10): 127-136 |
| | sugars, such as N-acetyllactosamine | |
| | (Galβ1-3GlcNAc or Galβ1-4GlcNAc); | |
| | secreted by epithelial cells in the thymus | |
| | and mediates T cell apoptosis; enhances | |
| | maturation of dendritic cells to secrete | |
| | inflammatory cytokines | |
| Leukemia inhibitory | induces terminal differentiation of myeloid | Annual Review Cell |
| factor (LIF) | leukemia cells, thus preventing their | Developmental |
| | continued growth; induces neuronal cell | Biology. 30: 647, |
| | differentiation and the stimulation of acute- | 2014 |
| | phase protein synthesis in hepatocytes. | |
| Human leukocyte | responsible for the immunomodulatory | Stem Cells. 2008 |
| antigen-G5 (HLA-G5) | properties of mesenchymal stem cells | Jan; 26(1): 212-22 |
| | (MSCs) | |
| hypoxia-inducible | a transcription factor responding to | Cancer Res. 2006 |
| factor-1alpha (HIF-2- | decreases in available oxygen in the | Jun15; 66(12): |
| alpha) | cellular environment; regulates | 6264-70 |
| | transcriptional activation of VEGF in | |
| | response to hypoxia | |
| anti-TL1A monoclonal | monoclonal antibodies against receptor | U.S. Pat. No. 8,263,743 |
| antibody | TNF superfamily member 15 (TNFSF15), | |
| | also known as TL1A; may be used for the | |
| | treatment of autoimmune inflammatory | |
| | diseases | |
| anti-integrin | antibody targeting/blocking integrin α4β7 | WO2012151248 |
| alpha4,beta7 | (LPAM-1, lymphocyte Peyer's patch | |
| | adhesion molecule 1); has gut-specific anti- | |
| | inflammatory activity; one example is | |
| | Vedolizumab (trade name Entyvio), which | |
| | is a monoclonal antibody developed by | |
| | Millennium Pharmaceuticals; may be used | |
| | for the treatment of inflammatory bowel | |
| | disease (IBD) | |
| anti-MAdCAM | antibody targeting mucosal vascular | WO2007007173 |
| | addressin cell adhesion molecule 1 | |
| | (MAdCAM-1), which is a ligand for α4β7 | |
| | integrin; one example is PF-547659, a | |
| | monoclonal antibody against MadCAM-1 | |
| | developed by Pfizer; may be used for the | |
| | treatment of inflammatory bowel disease | |
| | (IBD) | |
| anti-MMP9 | antibody against Matrix metallopeptidase 9 | WO2012027721 |
| | (MMP-9); inhibitory anti-MM9 (e.g., | |
| | humanized monoclonal antibody GS-5745 | |
| | developed by Gilead) is effective in reduce | |
| | tumor growth in colorectal cancer | |

### Additional Embodiments

The present disclosure encompasses the following embodiments presented as numbered paragraphs:

1. A mesenchymal stem cell engineered to produce (a) multiple effector molecules(e.g., cytokines, chemokines, antibodies, decoy receptors, enzymes, cell surface proteins, or combinations thereof), at least two of which modulate different cell types (e.g., modulate a function of different cell types) of the immune system or different functions of the same cell type, or (b) at least one homing molecule and at least one effector molecule that modulates a cell type of the immune system.
2. The mesenchymal stem cell of paragraph 1 comprising an engineered nucleic acid that comprises a promoter operably linked to a nucleotide sequence encoding an effector molecule.
3. The mesenchymal stem cell of paragraph 2 comprising an engineered nucleic acid that comprises a promoter operably linked to a nucleotide sequence encoding at least two effector molecules.
4. The mesenchymal stem cell of paragraph 1 comprising at least two engineered nucleic acids, each comprising a promoter operably linked to a nucleotide sequence encoding at least one effector molecule.
5. The mesenchymal stem cell of any one of paragraphs 1-4, wherein at least one effector molecule produced by the mesenchymal stem cell directly or indirectly modulates an innate immune cell, and wherein at least one effector molecule produced by the mesenchymal stem cell directly or indirectly modulates an adaptive immune cell.
6. The mesenchymal stem cell of paragraph 5, wherein the innate immune cell is selected from natural killer (NK) cells, NKT cells, mast cells, eosinophils, basophils, macrophages, neutrophils, and dendritic cells.
7. The mesenchymal stem cell of paragraph 5 or 6, wherein the adaptive immune cell is selected from T cells and B cells.
8. The mesenchymal stem cell of paragraph 7, wherein the T cells are selected from CD8⁺ T cells, CD4⁺ T cells, gamma-delta T cells, and T regulatory cells.
9. The mesenchymal stem cell of any one of paragraphs 1-4, wherein at least one effector molecule produced by the mesenchymal stem cell directly or indirectly modulates a pro-inflammatory cell, and wherein at least one effector molecule produced by the mesenchymal stem cell directly or indirectly modulates an anti-inflammatory cell.
10. The mesenchymal stem cell of paragraph 9, wherein the pro-inflammatory cell is selected from M1 macrophages, M1 mesenchymal stem cells, effector T cells, Th1 cells, Th17 cells, mature dendritic cells, and B cells.
11. The mesenchymal stem cell of paragraph 9 or 10, wherein the anti-inflammatory cell is selected from M2 macrophages, M2 mesenchymal stem cells, T regulatory cells, tolerogenic dendritic cells, regulatory B cells, and Tr1 cells.
12. The mesenchymal stem cell of any one of paragraphs 1-4, wherein at least one effector molecule produced by the mesenchymal stem cell directly or indirectly modulates a myeloid cell, and wherein at least one effector molecule produced by the mesenchymal stem cell directly or indirectly modulates a lymphoid cell.
13. The mesenchymal stem cell of paragraph 12, wherein the myeloid cell is selected from monocytes, macrophages, neutrophils, basophils, eosinophils, erythrocytes, dendritic cells, and megakaryocytes.
14. The mesenchymal stem cell of paragraph 12 or 13, wherein the lymphoid cell is selected from NK cells, T cells, and B cells.
15. The mesenchymal stem cell of any one of paragraphs 1-14, wherein the mesenchymal stem cell is engineered to produce a homing molecule.
16. The mesenchymal stem cell of paragraph 15, wherein the homing molecule is selected from: anti-integrin alpha4,beta7; anti-MAdCAM; CCR9; CXCR4; SDF1; MMP-2; CXCR1; and CXCR7.
17. The mesenchymal stem cell of any one of paragraphs 1-16, wherein the mesenchymal stem cell is engineered to produce a growth factor.
18. The mesenchymal stem cell of paragraph 17, wherein the growth factor is selected from: PDGF, FGF, EGF, and BMP.
19. The mesenchymal stem cell of any one of paragraphs 2-18, wherein the promoter is an inducible promoter.
20. The mesenchymal stem cell of any one of paragraphs 2-18, wherein the promoter is a CMV promoter, an EF1a promoter, an EFS promoter, a MND promoter, a PGK promoter, a SFFV promoter, a SV40 promoter, or a UbC promoter.
21. The mesenchymal stem cell of any one of paragraphs 2-18, wherein the promoter is a synthetic promoter.
22. The mesenchymal stem cell of any one of paragraphs 2-21, wherein the synthetic promoter comprises a transcription factor binding domain.
23. The mesenchymal stem cell of any one of paragraphs 1-22, wherein the at least one effector molecule is selected from PD-L1 (B7H1), IL-1RA, soluble IFNR, ustekinumab, p75 of TNFR, anti-TNFalpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, and CCL22.
24. The mesenchymal stem cell of any one of paragraphs 2-22, wherein the promoter is modulated by an immune cell (e.g., a product of an immune cell, e.g., a cytokine or chemokine), and wherein at least one effector molecule produced by the mesenchymal stem cell decreases expression of an inflammatory cytokine or activity of an inflammatory cytokine.
25. The mesenchymal stem cell of paragraph 24, wherein the immune cell is selected from T cells, Th1 cells, Th17 cells, and M1 macrophage cells that secrete IFN-gamma, IL-17A, or TNF-alpha.
26. The mesenchymal stem cell of paragraph 25, wherein the promoter is responsive to IFN-gamma, IL-17A, or TNF-alpha.
27. The mesenchymal stem cell of any one of paragraphs 24-26, wherein the promoter comprises a response element selected from an interferon-gamma-activated sequence (GAS), an interferon-stimulated response element (ISRE), and a NF-kappaB response element.
28. The mesenchymal stem cell of any one of paragraphs 2-22, wherein the promoter is activated in the presence of IFN-gamma, IL-17A, TNF-alpha, or IL-6, and wherein at least one effector molecule produced by the mesenchymal stem cell decreases expression of an inflammatory cytokine or activity of an inflammatory cytokine.
29. The mesenchymal stem cell of paragraph 28, wherein the promoter comprises a response element selected from GAS, an ISRE, a NF-kappaB response element, and a STAT3 response element.
30. The mesenchymal stem cell of any one of paragraphs 2-22, wherein the promoter is activated under hypoxic conditions, and wherein at least one effector molecule produced by the mesenchymal stem cell decreases expression of an inflammatory cytokine or activity of an inflammatory cytokine.
31. The mesenchymal stem cell of paragraph 30, wherein the promoter comprises a hypoxia responsive element (HRE).
32. The mesenchymal stem cell of paragraph 31, wherein the promoter is responsive to HIF-1a transcription factor.
33. The mesenchymal stem cell of any one of paragraphs 24-32, wherein at least one effector molecule produced by the mesenchymal stem cell is selected from PD-L1 (B7H1), IL-1RA, soluble IFNR, ustekinumab, p75 of TNFR, anti-TNF-alpha Nanobody®, adalimumab, MEDI2070, IL-10, IL-11, IL-13, IL-4, IL-35, IL-22, IDO, iNOS, COX2, HO1, TSG-6, Galectin-9, LIF, HLA-G5, HIF-2-alpha, anti-TL1A monoclonal antibody, anti-integrin alpha4,beta7, anti-MAdCAM, anti-MMP9, TGF-beta, IL-33, and CCL22.
34. The mesenchymal stem cell of any one of paragraphs 24-33, wherein the inflammatory cytokine is selected from: IFN-gamma, IL-17A, IL-6, IFN-alpha, TNF-alpha, IL-1b, IL-8, IL-12(p70), IL-18, and IL-23.
35. The mesenchymal stem cell of any one of paragraphs 2-22, wherein the promoter is modulated by an immune cell (e.g., a product of an immune cell, e.g., a cytokine or chemokine), and wherein at least one effector molecule produced by the mesenchymal stem cell is an anti-inflammatory cytokine, or wherein at least one effector molecule produced by the mesenchymal stem cell increases expression of an anti-inflammatory cytokine or activity of an anti-inflammatory cytokine.
36. The mesenchymal stem cell of paragraph 35, wherein the immune cell is selected from T cells, Th1 cells, Th17 cells, and M1 macrophage cells that secrete IFN-gamma, IL-17A, or TNF-alpha.
37. The mesenchymal stem cell of paragraph 36, wherein the promoter is responsive to IFN-gamma, IL-17A, or TNF-alpha.
38. The mesenchymal stem cell of any one of paragraphs 35-37, wherein the promoter comprises a response element selected from an interferon-gamma-activated sequence (GAS), an interferon-stimulated response element (ISRE), and a NF-kappaB response element.
39. The mesenchymal stem cell of any one of paragraphs 2-22, wherein the promoter is activated in the presence of IFN-gamma, IL-17A or TNF-alpha, and wherein at least one effector molecule produced by the mesenchymal stem cell is an anti-inflammatory cytokine, or wherein at least one effector molecule produced by the mesenchymal stem cell increases expression of an anti-inflammatory cytokine or activity of an anti-inflammatory cytokine.
40. The mesenchymal stem cell of paragraph 39, wherein the promoter comprises a response element selected from GAS, an ISRE, a NF-kappaB response element, and a STAT3 response element.
41. The mesenchymal stem cell of any one of paragraphs 2-22, wherein the promoter is activated under hypoxic conditions, and wherein at least one effector molecule produced by the mesenchymal stem cell is an anti-inflammatory cytokine, or wherein at least one effector molecule produced by the mesenchymal stem cell increases expression of an anti-inflammatory cytokine or activity of an anti-inflammatory cytokine.
42. The mesenchymal stem cell of paragraph 41, wherein the promoter comprises a hypoxia responsive element (HRE).
43. The mesenchymal stem cell of paragraph 42, wherein the promoter is responsive to HIF-1a transcription factor.
44. The mesenchymal stem cell of any one of paragraphs 35-43, wherein the anti-inflammatory cytokine is selected from: IL-4, IL-5, IL-10, IL-13, CCL2 and IL-33.
45. The mesenchymal stem cell of any one of paragraphs 2-22, wherein the promoter is modulated by an immune cell (e.g., a product of an immune cell, e.g., a cytokine or chemokine), and wherein at least one effector molecule produced by the mesenchymal stem cell promotes conversion of T regulatory cells, promotes stability of T regulatory cells, increases the prevalence of T regulatory cells, or increases recruitment of T regulatory cells.
46. The mesenchymal stem cell of paragraph 45, wherein the immune cell is selected from T cells, Th1 cells, Th17 cells, and M1 macrophage cells that secrete IFN-gamma, IL-17A, or TNF-alpha.
47. The mesenchymal stem cell of paragraph 46, wherein the promoter is responsive to IFN-gamma, IL-17A, or TNF-alpha.
48. The mesenchymal stem cell of any one of paragraphs 45-47, wherein the promoter comprises a response element selected from an interferon-gamma-activated sequence (GAS), an interferon-stimulated response element (ISRE), and a NF-kappaB response element.
49. The mesenchymal stem cell of any one of paragraphs 2-22, wherein the promoter is activated in the presence of IFN-gamma, IL-17A or TNF-alpha, and wherein the effector molecule promotes conversion of T regulatory cells, promotes stability of T regulatory cells, increases the prevalence of T regulatory cells, or increases recruitment of T regulatory cells.
50. The mesenchymal stem cell of paragraph 49, wherein the promoter comprises a response element selected from GAS, an ISRE, a NF-kappaB response element, and a STAT3 response element.
51. The mesenchymal stem cell of any one of paragraphs 2-22, wherein the promoter is activated under hypoxic conditions, and wherein at least one effector molecule produced by the mesenchymal stem cell promotes conversion of T regulatory cells, promotes stability of T regulatory cells, increases the prevalence of T regulatory cells, or increases recruitment of T regulatory cells.
52. The mesenchymal stem cell of paragraph 51, wherein the promoter comprises a hypoxia responsive element (HRE).
53. The mesenchymal stem cell of paragraph 52, wherein the promoter is responsive to HIF-1a transcription factor.
54. The mesenchymal stem cell of any one of paragraphs 45-53, wherein at least one effector molecule produced by the mesenchymal stem cell is selected from TGF-beta, tocilizumab (anti-IL6), indoleamine 2,3-dioxygenase (IDO), IL-35, PD-L1, IL-2 and IL-2 variants.
55. The mesenchymal stem cell of any one of paragraphs 1-22, wherein at least one effector molecule produced by the mesenchymal stem cell modulates a molecule natively produced by the mesenchymal stem cell.
56. The mesenchymal stem cell of paragraph 55 wherein at least one effector molecule produced by the mesenchymal stem cell increases or decreases activity of the molecule natively produced by the mesenchymal stem cell.
57. The mesenchymal stem cell of paragraph 56, wherein expression and/or activity of at least one effector molecule produced by the mesenchymal stem cell and the molecule natively produced by the mesenchymal stem cell are regulated by the same input signal.
58. The mesenchymal stem cell of paragraph 56, wherein expression and/or activity of at least one effector molecule produced by the mesenchymal stem cell and the molecule natively produced by the mesenchymal stem cell are regulated by different input signals.
59. The mesenchymal stem cell of any one of paragraphs 1-22, wherein at least one effector molecule produced by the mesenchymal stem cell complements a molecule natively produced by the mesenchymal stem cell.
60. The mesenchymal stem cell of any one of paragraphs 1-22, wherein at least one effector molecule produced by the mesenchymal stem cell modulates and complements a molecule natively produced by the mesenchymal stem cell.
61. A method comprising culturing the mesenchymal stem cell of any one of paragraphs 1-60 to produce the effector molecules.
62. A method comprising delivering to a subject the mesenchymal stem cell of any one of paragraphs 1-60 to produce in vivo at least one effector molecule produced by the mesenchymal stem cell.
63. A method of treating an inflammatory bowel disease, comprising delivering to subject diagnosed with an inflammatory bowel disease the mesenchymal stem cell of any one of paragraphs 1-60.
64. The method of paragraph 63, wherein the inflammatory bowel disease is ulcerative colitis or Crohn's disease.
65. A method of producing a multifunctional immunomodulatory cell, comprising

(a) delivering to a mesenchymal stem cell at least one engineered nucleic acid encoding at least two effector molecules, or

(b) delivering to a mesenchymal stem cell at least two engineered nucleic acids, each encoding at least one effector molecule,

wherein each effector molecule modulates a different cell type of the immune system or modulates different functions of a cell.

66. A method of modulating multiple cell types of the immune system of a subject, comprising delivering to the subject at least two mesenchymal stem cells, each engineered to produce an effector molecule, wherein at least two of the effector molecules modulate different cell types of the immune system.
67. The mesenchymal stem cell of any one of claim 1-60, wherein the effector molecule is an anti-inflammatory molecule and the homing molecule is selected from CXCR4, CCR2, CCR9, and GPR15 (other homing molecules describe herein may be used).
68. The mesenchymal stem cell of claim 67, wherein the anti-inflammatory molecule is IL-4.
69. The mesenchymal stem cell of claim 67, wherein the anti-inflammatory molecule is IL-10.
70. The mesenchymal stem cell of claim 67, wherein the anti-inflammatory molecule is IL-35.
71. The mesenchymal stem cell of claim 67, wherein the anti-inflammatory molecule is PD-L1-Ig.
72. The mesenchymal stem cell of claim 67, wherein the anti-inflammatory molecule is anti-TNF-alpha.
73. The mesenchymal stem cell of claim 67, wherein the anti-inflammatory molecule is indoleamine 2,3-dioxygenase (IDO).
74. The mesenchymal stem cell of claim 67, wherein the anti-inflammatory molecule is alpha-1 antitrypsin.
75. The mesenchymal stem cell of claim 67, wherein the anti-inflammatory molecule is a wound-healing molecule.
76. The mesenchymal stem cell of claim 75, wherein the would-healing molecule is IL-22.
77. The mesenchymal stem cell of claim 75, wherein the would-healing molecule is IL-19.
78. The mesenchymal stem cell of claim 75, wherein the would-healing molecule is IL-20
79. The mesenchymal stem cell of any one of claims 67-78, wherein the homing molecule is CXCR4.
80. The mesenchymal stem cell of any one of claims 67-79, wherein the homing molecule is CCR2.
81. The mesenchymal stem cell of any one of claims 67-80, wherein the homing molecule is CCR9.
82. The mesenchymal stem cell of any one of claims 67-81, wherein the homing molecule is GPR15.
83. The mesenchymal stem cell of any one of claim 1-60, wherein one of the effector molecules is a cytokine (e.g., an anti-inflammatory cytokine) and one of the effector molecules is a chemokine (e.g., a chemokine that recruits anti-inflammatory cells).
84. The mesenchymal stem cell of any one of claims 1-60, wherein the mesenchymal stem cell is not engineered to express a chemokine (e.g., does not include an engineered nucleic acid encoding a chemokine).

### EXAMPLES

### Example 1

Mesenchymal stem cells (MSCs) were nucleofected with various expression vectors selected from the following:

pmaxGFP (LONZA® positive control)

CMV-IL4 expression vector (no fluorescent reporter)

CMV-IL10 expression vector (no fluorescent reporter)

2× negative controls (no DNA, untransfected)

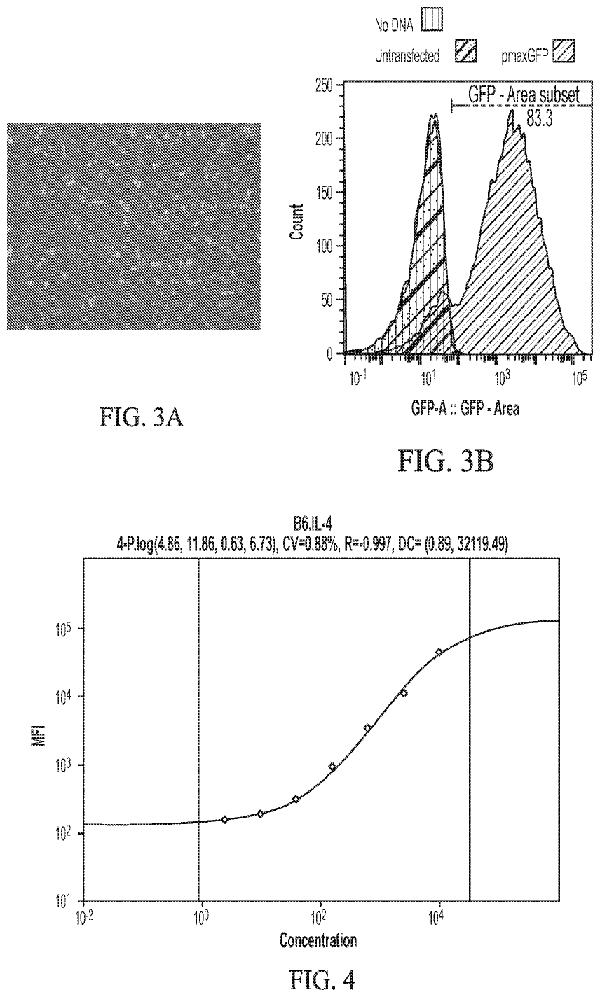

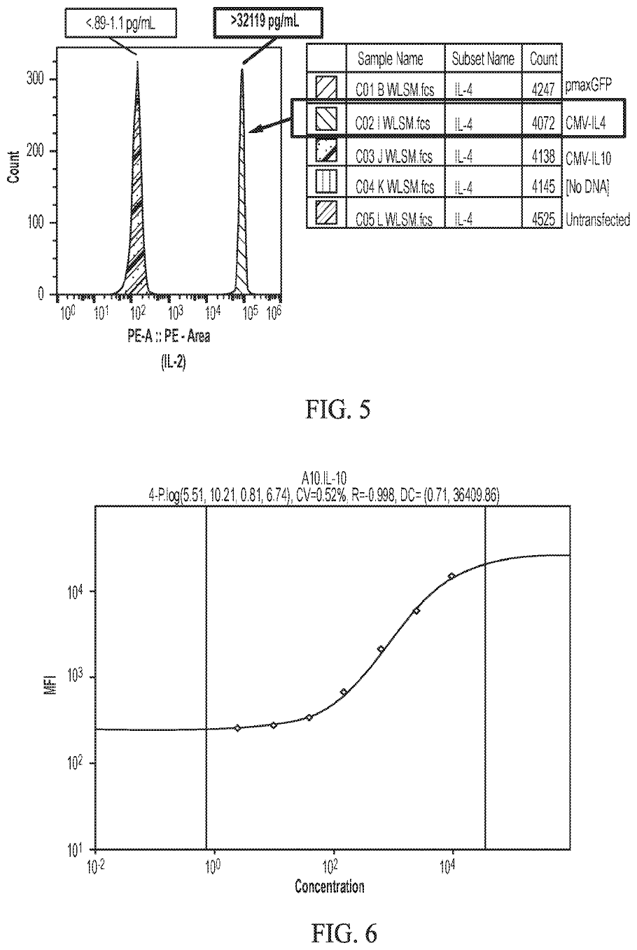

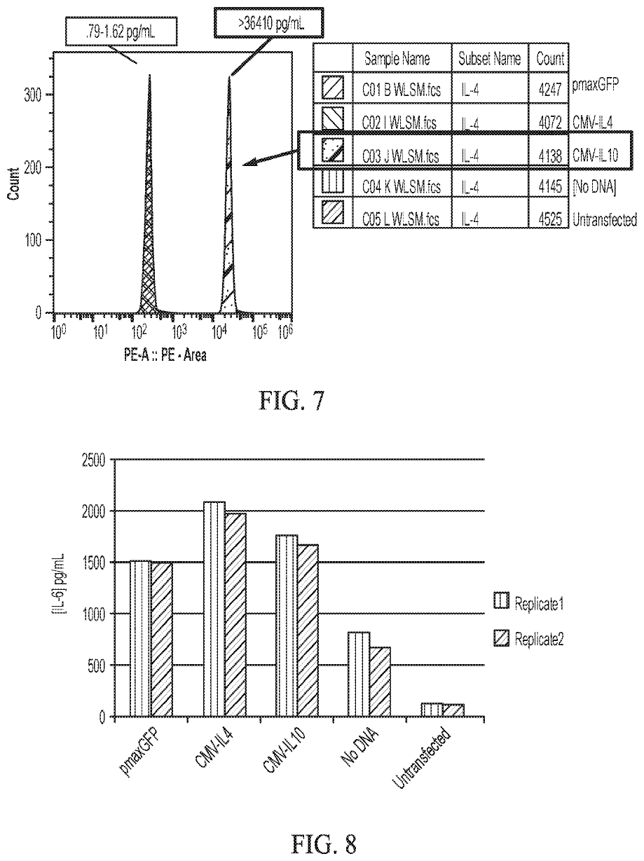

Supernatant from the MSCs was collected 24 hours after nucleofection and was frozen. The supernatant was subsequently analyzed using a BIOLEGEND® kit, quantifying seven cytokines IL-4, IL-5, IL-6, IL-10, IL-13, IL-17A, and IFN-gamma. The results from these experiments are shown and described in FIGS. 3A-8.

### Example 2

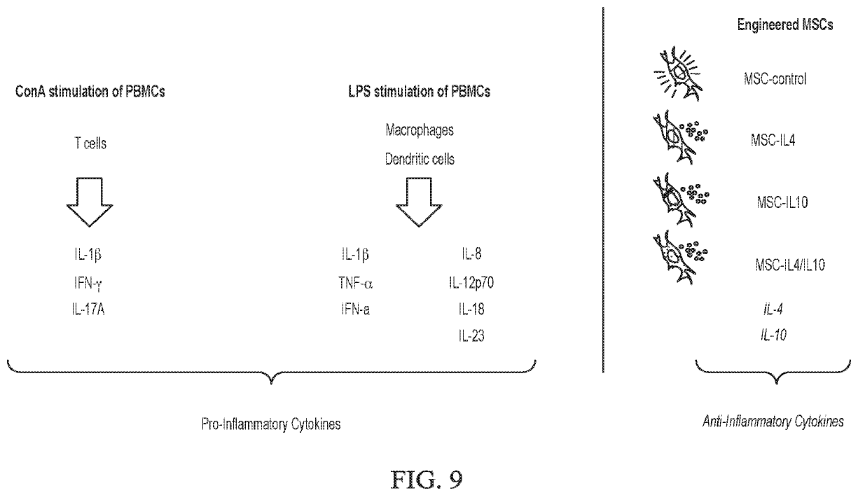

PBMCs were stimulated with either concanavalin A (ConA) or lipopolysaccharide (LPS) to induce production of various pro-inflammatory cytokines. Engineered MSCs expressing anti-inflammatory cytokines IL-4, IL-10, both IL-4/IL-10, or a control were generated for use in co-culture experiments with the stimulated PBMCs (schematized in FIG. 9).

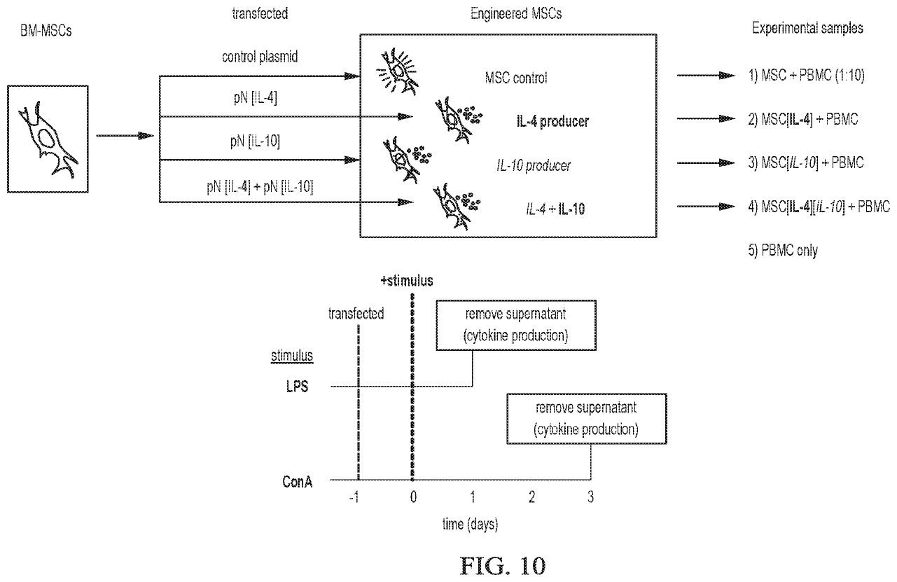

Bone-marrow derived MSCs (BM-MSCS) were transfected with control plasmid, IL-4 expression plasmid (pN [IL-4]), IL-10 expression plasmid (pN [IL-10]), or a combination of both IL-4 and IL-10 expression plasmids each at half the amount of the single plasmids. Following transfection the engineered MSCs were rested overnight. The following day PBMCs were stimulated with LPS [1 μg/ml] or ConA [2.5 μg/ml] at 25,000 cells per well in a tissue culture-treated 96-well flat-bottom plate with experimental samples containing of PBMCs only or PBMCs co-cultured with MSCs at 1:10 ratio (25,000 PBMCs with 2,500 MSCs) or where indicated at 1:160 ratio (16-fold dil) with appropriately diluted numbers of MSCs. Supernatants were collected at Day1 post-stimulation for the LPS set and at Day3 post-stimulation for the ConA set (schematized in FIG. 10). Supernatant cytokines were measured by flow cytometry using multi-analyte bead-antibody conjugated cytokine capture and detection assays. All conditions were conducted as triplicate biological replicates.

MSCs transfected with IL-4, IL-10, or both expression plasmids produced the expected anti-inflammatory cytokine as measured in supernatants when co-cultured with stimulated PBMCs (FIG. 11). P=Stimulated PBMCs only; P+M(cntl)=Stimulated PBMCs co-cultured with MSCs transfected with control plasmid; P+M(4)=Stimulated PBMCs co-cultured with MSCs transfected with IL-4 expression plasmid; P+M(10)=Stimulated PBMCs co-cultured with MSCs transfected with IL-10 expression plasmid; P+M(4/10)=Stimulated PBMCs co-cultured with MSCs transfected with IL-4 and IL-10 expression plasmids at half the amount of the single plasmids. Bars represent the mean of biological triplicates, error bars indicate standard error of the mean (S.E.M.).

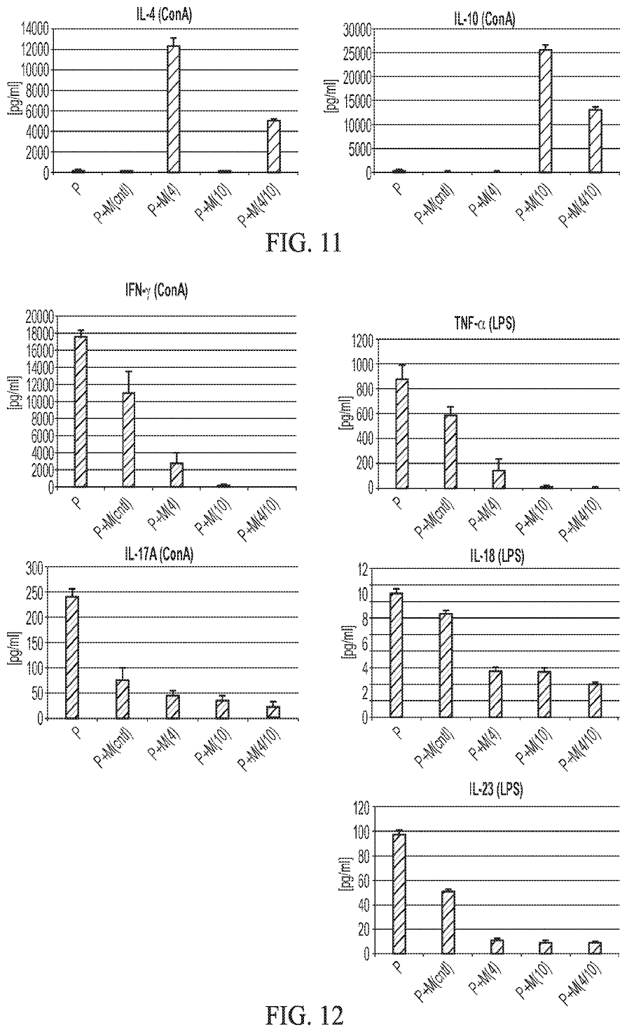

MSCs transfected with IL-4, IL-10, or both expression plasmids demonstrated increased inhibitory capacity compared to control MSCs in suppression of pro-inflammatory cytokines as measured in supernatants when co-cultured with stimulated PBMCs (FIG. 12).

MSCs transfected with IL-4, IL-10, or both expression plasmids demonstrate the ability to inhibit pro-inflammatory cytokine production compared to a lack of inhibition by control MSCs as measured in supernatants when co-cultured with stimulated PBMCs (FIG. 13).

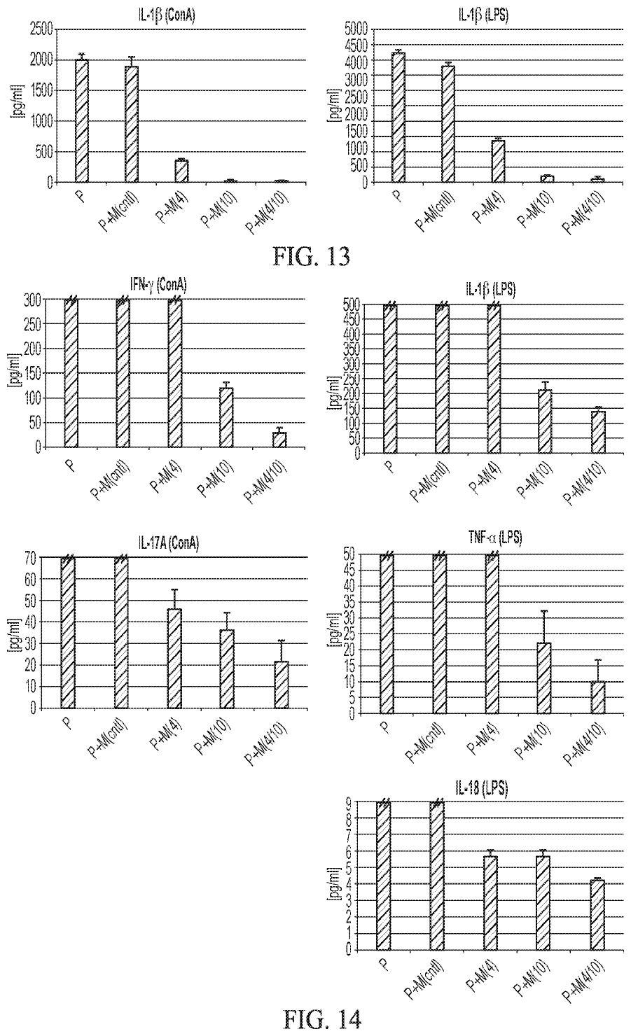

MSCs transfected with the combination of IL-4 and IL-10 expression plasmids showed increased inhibitory capacity compared to engineered MSCs transfected with either IL-4 or IL-10 expression plasmids alone in suppression of pro-inflammatory cytokines as measured in supernatants when co-cultured with stimulated PBMCs (FIG. 14).

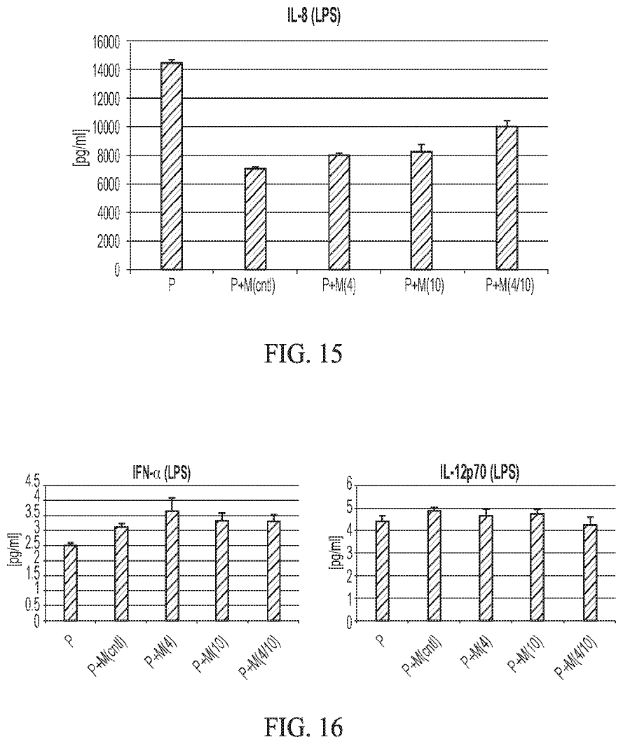

MSCs transfected with IL-4, IL-10, or both expression plasmids did not demonstrate increased effectiveness compared to control MSCs to inhibit pro-inflammatory cytokine production in some cases as measured in supernatants when co-cultured with stimulated PBMCs (FIG. 15).

MSCs transfected with IL-4, IL-10, both expression plasmids, or control MSCs did not demonstrate the ability to inhibit pro-inflammatory cytokine production in some cases as measured in supernatants when co-cultured with stimulated PBMCs (FIG. 16).

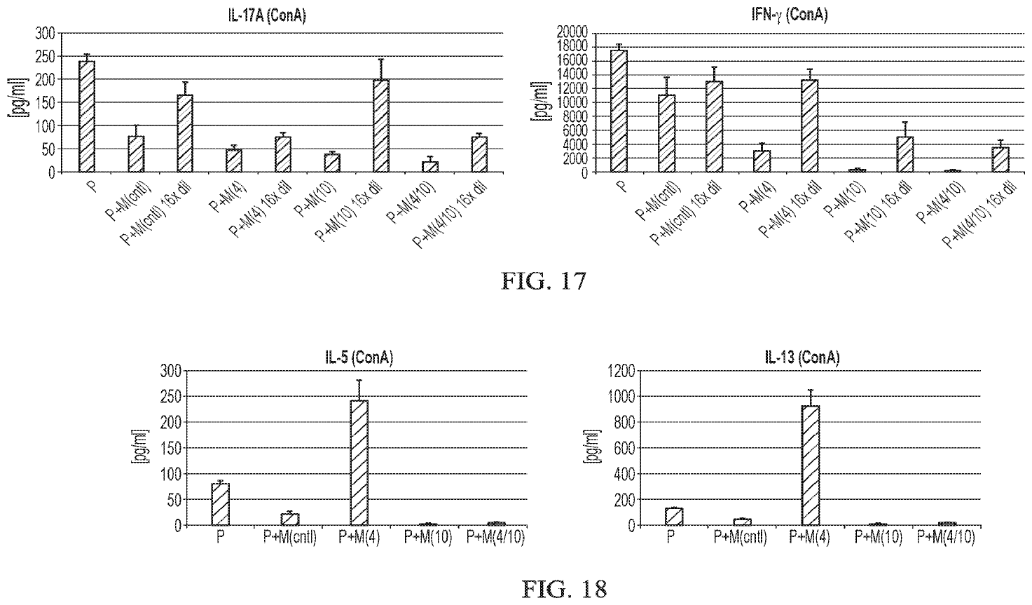

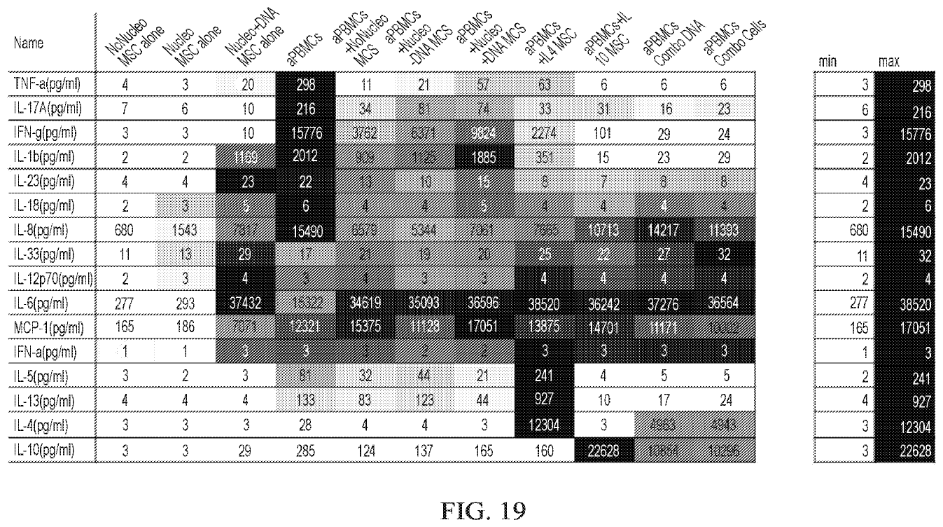

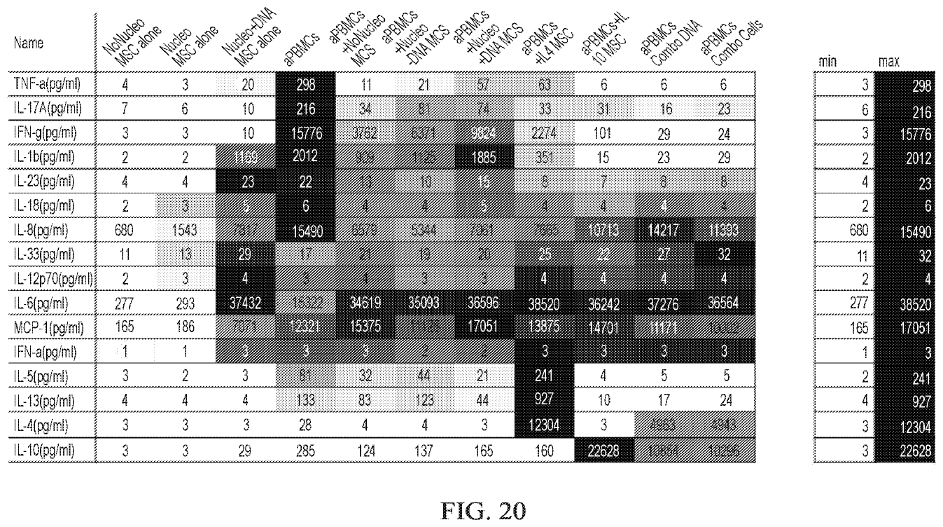

MSCs transfected with IL-4, IL-10, or both expression plasmids demonstrated the ability to inhibit pro-inflammatory cytokine production even when co-cultured at 16-fold less MSCs (16× dil) than standard MSC co-culture conditions compared to diminished inhibitory capacity of control MSCs at 16-fold less as measured in supernatants when co-cultured with stimulated PBMCs. The engineered MSC combination of IL-4/IL-10 matched the inhibitory capacity of whichever single IL-4 or IL-10 engineered MSC showed the greater inhibition capacity when MSCs were diluted 16-fold (FIG. 17).

MSCs transfected with IL-4 expression plasmid induced the production of other anti-inflammatory cytokines compared to control MSCs, MSC(IL-10), or combination MSC(IL4/IL-10) as measured in supernatants when co-cultured with stimulated PBMCs (FIG. 18).

### Example 3

The following experiments demonstrate that: (1) different MSC sources have varying intrinsic immune inhibiting capacity; (2) MSCs co-cultured with human CD4+ T cells can induce a regulatory T cell immunophenotype.

Human CD4+ T cells were isolated by magnetic bead sorting from PBMC, stained with CFSE proliferation dye, and stimulated using anti-CD3/28 Dynabeads, with or without MSCs at ratios of 1:10, 1:40, and 1:160 to CD4+ T cells. After 3 days of stimulation CD4+ T cells were harvested and analyzed by flow cytometry to assess proliferation via CFSE dye dilution. Flow diagrams depict CFSE histograms of the various conditions with MSC sources (adipose, bone marrow, or umbilical cord) co-cultured at ratios of 1:10, 1:40, and 1:160 (data not shown). All conditions were done as three biological replicates. This data showed that different MSC sources have varying intrinsic immune inhibiting capacity.

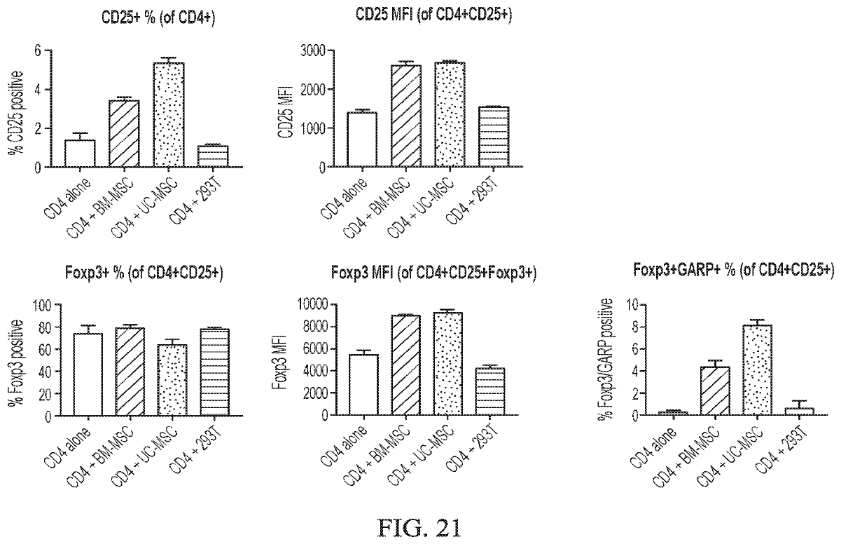

Next, magnetic bead-isolated CD4+ T cells from human PMBCs were cultured with human bone-marrow MSCs, human umbilical cord MSCs, or 293T cells. PMBCs alone were used as a control. 1×10⁶ CD4+ T cells were cultured with 1×10⁵ MSCs or 293T cells for 3 days and then stained for flow cytometry. CD4+ cells were first gated by size (FSC) and granularity (SSC) and then by surface expression of CD4, and expression of CD25 percentage and mean fluorescence intensity (MFI) measured (flow cytometry dot plots not shown). CD4+CD25+ gated cells were also analyzed for expression of intracellular stained Foxp3 and surface glycoprotein A repetitions predominant (GARP). Bar graphs show the percentage positive and MFI of the various culture conditions (FIG. 21). Three biological replicates were conducted per culture condition. This data showed that MSCs co-cultured with human CD4+ T cells can induce a regulatory T cell immunophenotype.

### Example 4

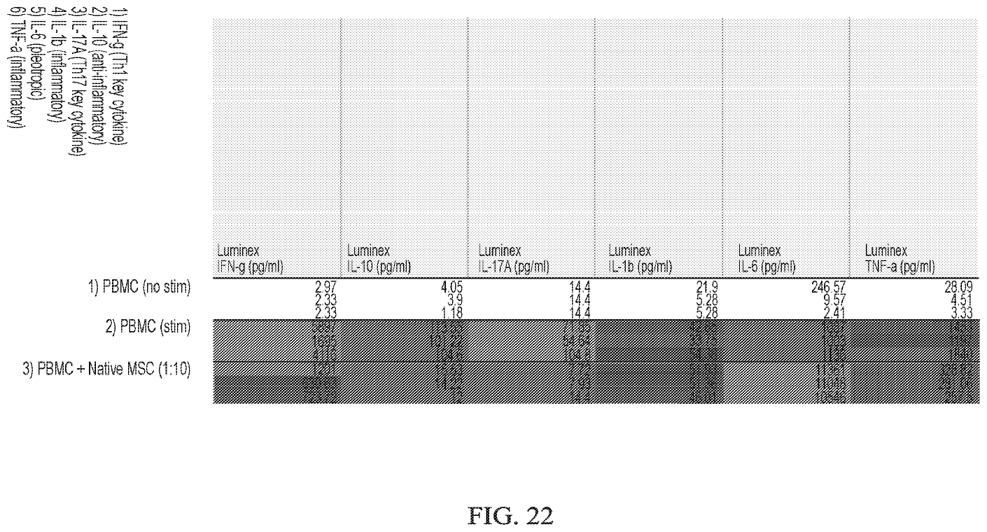

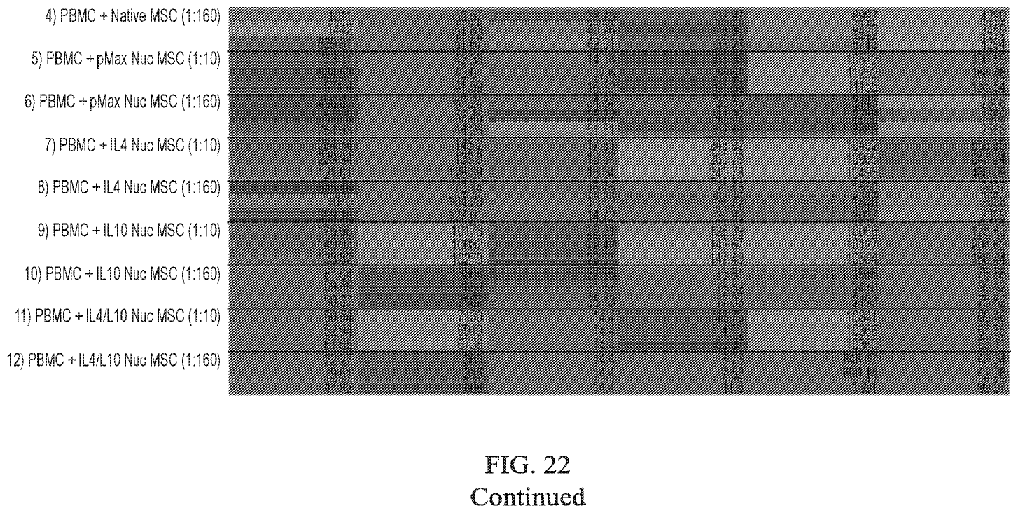

The following experiments demonstrate that T cell stimulation-induced inflammatory cytokines are inhibited by MSCs engineered to secrete anti-inflammatory cytokine IL-4 or IL-10. T cells from PBMC were stimulated using anti-CD3/28 Dynabeads and cultured alone or with bone-marrow MSCs at the indicated ratios for 3 days then supernatant collected to assay cytokines. MSCs were native, or nucleofected with control pMax vector, IL-4, or IL-10, and co-cultured as indicated. Combination IL-4/IL-10 condition was with an equal mix of nucleofected IL-4 and IL-10 MSCs. Supernatants were assayed by Luminex cytokine bead arrays to the indicated cytokine set that includes IFN-gamma, IL-10, IL-17A, IL-1beta, IL-6, and TNF-alpha. Three Luminex technical replicates were conducted per culture condition. The results from this experiment are shown in FIG. 22.

### Example 5

To demonstrate that injected engineered MSCs expressing cytokines are able to alter immune cell populations in mice, 3% Dextran sulfate sodium (DSS) was administered to C57BL/6 mice in drinking water for 2 days to induce colitis, then 1×10⁶ MSCs engineered by nucleofection to express mouse IL-4 or IL-10 were administered via intraperitoneal injection. After 3 additional days mice were sacrificed and peritoneal cells isolated and stained by flow cytometry for F4/80 marker expression to mark macrophages. Administered MSC-IL-10 engineered cells led to a slight increase in macrophages while MSC-IL-4 engineered cells led to a decrease in macrophages within the peritoneal cell population (data not shown).

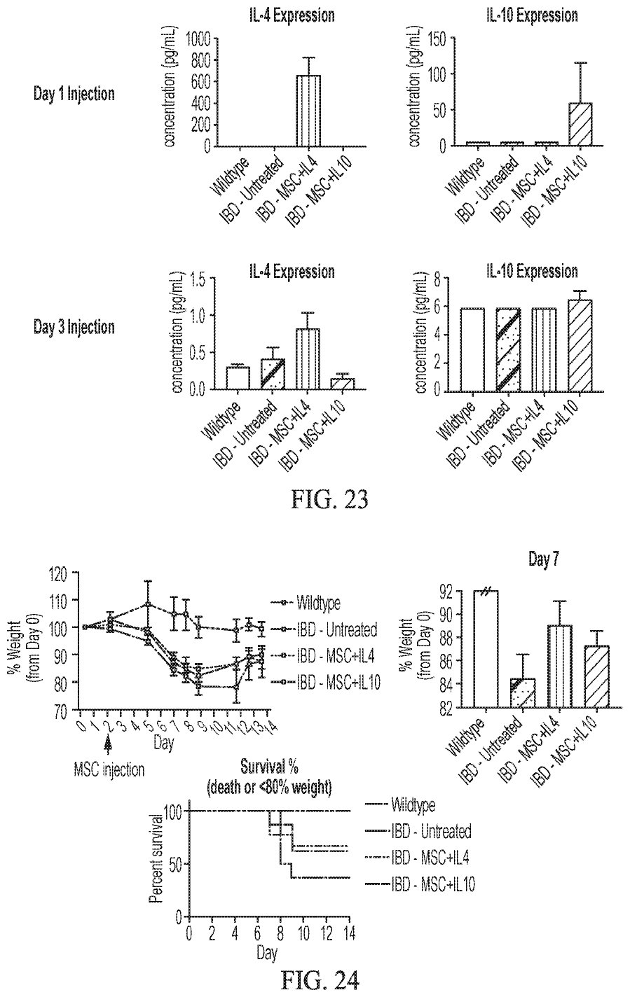

Next, colitis was again induced in another cohort of mice, as described above, then 1×10⁶ MSCs engineered by nucleofection to express mouse IL-4 or IL-10 were administered via intraperitoneal injection. After 1 or 3 additional days mice were sacrificed and peritoneal fluid isolated and assayed for cytokine expression by Luminex cytokine bead multi-array. Each bar represents an average of 2-5 mice per group collected with error bars representing standard error of means (SEM) (FIG. 23). These experiments demonstrate that injected engineered MSCs expressing cytokines maintained cytokine expression in vivo.

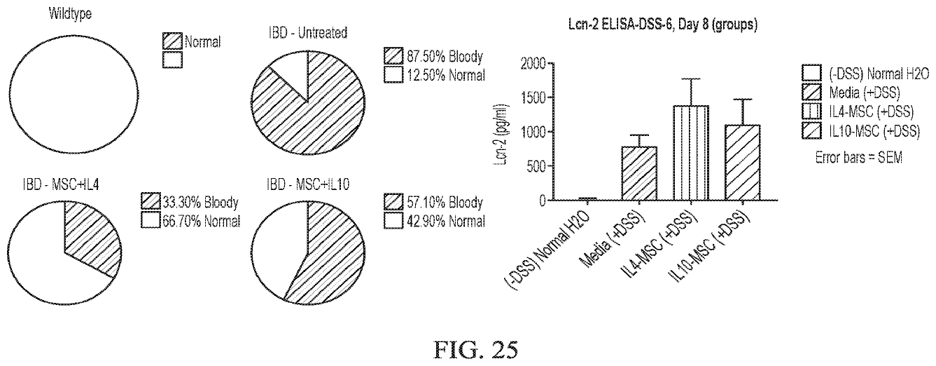

To demonstrate improved weight and survival from injected engineered MSCs in DSS colitis mice, colitis was induced in another cohort of mice, as described above, then 1×10⁶ MSCs engineered by nucleofection to express mouse IL-4 or IL-10 were administered via intraperitoneal injection. Mice weight was recorded as was survival scored as death or weight <80% starting weight. Injection cohorts and measurements were conducted in a double-blinded manner (FIG. 24). Each cohort represents an average of 8 mice per group with error bars representing standard error of means (SEM). Similar experiments demonstrated improved bloody stool and inflammatory lipocalin-2 levels from injected engineered MSCs in DSS colitis mice (FIG. 25). Mice bloody stool was recorded on Day 8 of DSS start, and stool was processed for protein to measure lipocalin-2 (Lcn-2) levels by ELISA. Injection cohorts and measurements were conducted in a double-blinded manner. Each cohort represents an average of 8 mice per group with error bars representing standard error of means (SEM).

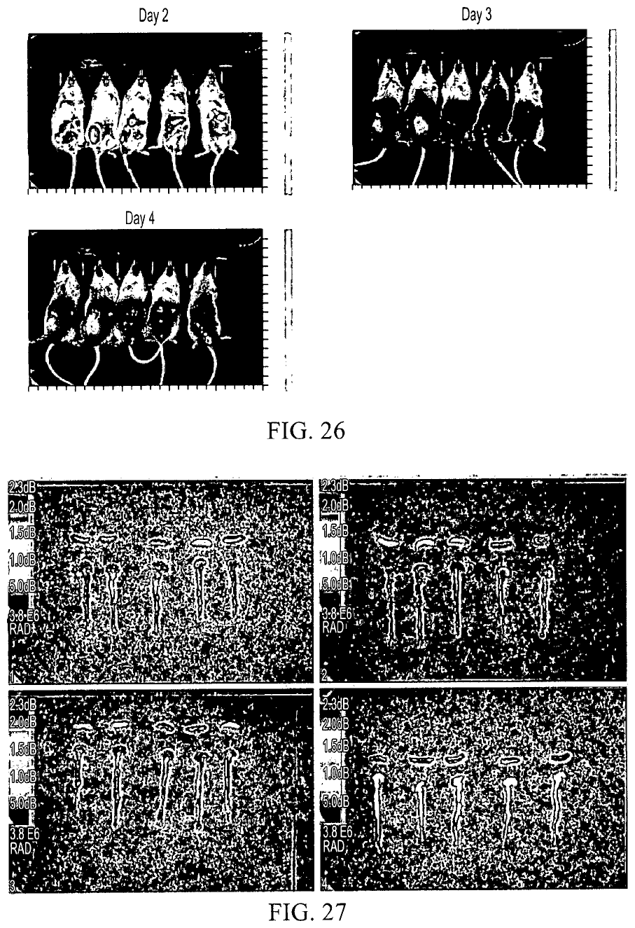

The following experiments show MSC biodistribution and persistence in DSS colitis mice (FIG. 26), and specifically MSC biodistribution and persistence within the colon and spleen in DSS colitis mice (FIG. 27). 3% Dextran sulfate sodium (DSS) was administered to C57BL/6 mice in drinking water for 2 days to induce colitis then 1×10⁶ MSCs engineered by nucleofection to express mouse IL-4, IL-10, or control GFP (pMax). For general distribution studies, MSCs were stained with in vivo fluorescence tracking dye XenoLight DiR and administered via intraperitoneal injection. Mice live imaging was performed on excitation and emission channels for DiR fluorescence imaging on a Spectral Instruments Ami imager on DSS Day 2 (day of MSC injection), Day 3 (1 day after MSC injection), and Day 4 (2 days after MSC injection). Fluorescence was measured as photons per seconds (FIG. 26). For organ-specific distribution studies, mice were sacrificed on Day 4 (2 days after MSC injection) and colon and spleen dissected and imaging was performed on excitation and emission channels for DiR fluorescence imaging on a Spectral Instruments Ami imager. Top-left is MSC-GFP, top-right is MSC-IL4, bottom-left is MSC-IL10, bottom-right is no MSC. Fluorescence was measured as photons per seconds (FIG. 27).

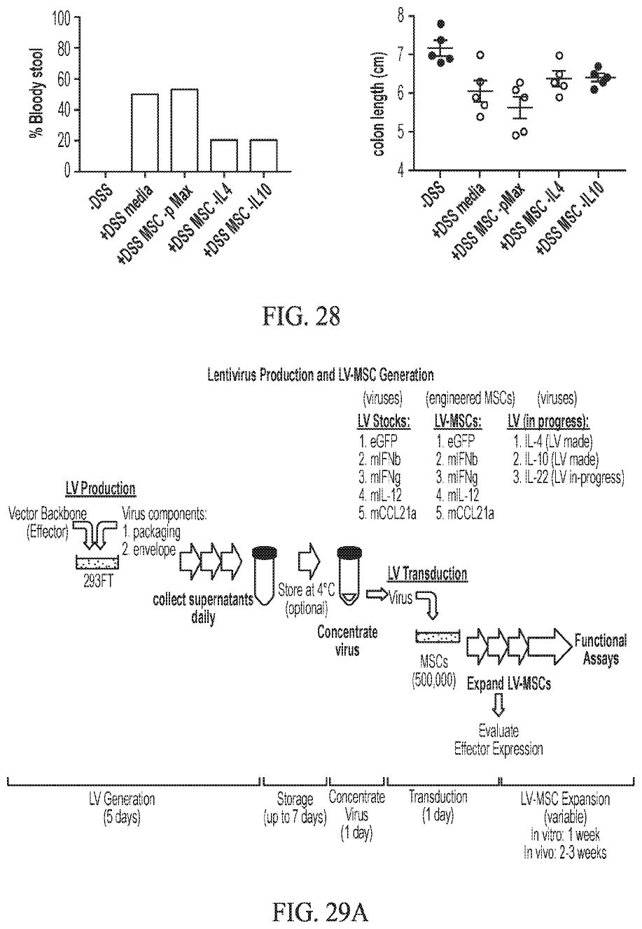

To show improved bloody stool and colon lengths from injected engineered MSCs specific to anti-inflammatory cytokines in DSS colitis mice, colitis was induced in mice with DSS, as described above, then 1×10⁶ MSCs engineered by nucleofection to express mouse IL-4, IL-10, or control GFP (pMax) were administered via intraperitoneal injection. Mice bloody stool was recorded on Day 4 of DSS start. Colon lengths were measured on Day 7 after sacrifice of mice. Injection cohorts and measurements were conducted in a double-blinded manner. Each cohort represents an average of 5 mice per group with error bars representing standard error of means (SEM) (FIG. 28).

### Example 6

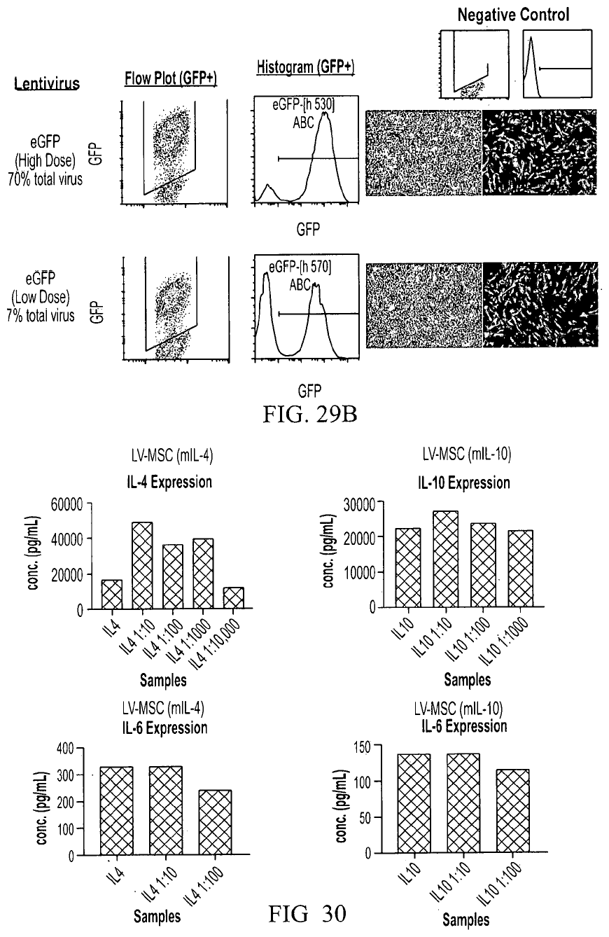

For the following experiments, lentiviruses were used to transduce MSCs to generate engineered MSCs. The workflow is shown in FIGS. 29A and 34B.

MSCs were transduced using the workflow shown in FIG. 29A and supernatant harvest 24 hours after transduction and assayed for cytokine expression by Luminex cytokine bead multi-array. Lentivral mouse IL-4 and IL-10 transduction resulted in high expression of these proteins secreted in the supernatant while baseline IL-6 levels were unaffected. Mouse IL-17A, TNF-alpha, IL-1beta, and IFN-gamma were below the limit of detection. Bars represent duplicate technical replicates. FIG. 30 shows lentiviral transduction to generate engineered MSCs resulted in desired cytokine expression absent inflammatory cytokine expression.

### Example 7

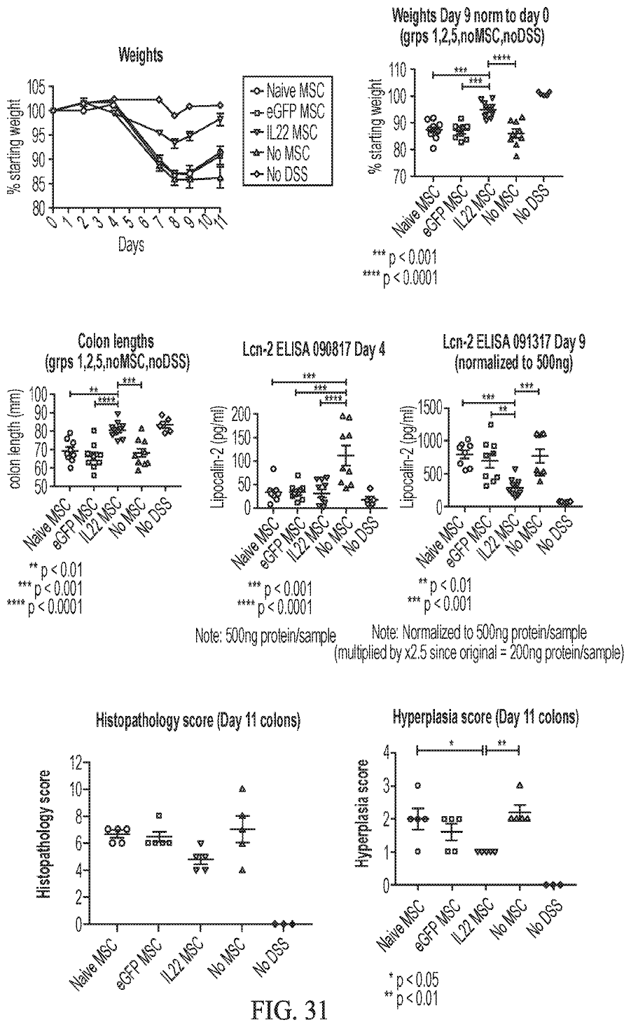

For this set of experiments, 3% Dextran sulfate sodium (DSS) was administered to C57BL/6 mice in drinking water for 2 days to induce colitis, then 4×10⁶ MSCs engineered by lentiviral transduction to express mouse IL-22 or control GFP were administered via intraperitoneal injection. Colon lengths were measured on Day 11 upon sacrifice of mice. Stool protein was collected at Day 4 or Day 9 and lipocalin-2 (Lcn-2) levels measured by ELISA. Colon was dissected, fixed in 10% formalin, and longitudinal slices of the entire colon embedded and stained with haemotoxylin and eosin (H&E). Scoring was conducted by a blinded animal pathologist with experience in mouse models of colitis. Histopathology scoring included severity of inflammation, percent of area affected by inflammation, ulceration, fibrosis of the lamina propria leading to separation of the glands, and edema of the mucosa and/or submucosa. Hyperplasia scoring included degree of hyperplasia and percent of area affected by hyperplastic changes. Injection cohorts and measurements were conducted in a double-blinded manner. Each cohort represents an average of 8-10 mice per group with error bars representing standard error of means (SEM). FIG. 31 shows improved weight, colon length, lipocalin-2 levels, and colon histopathology and hyperplasia scoring from injected lentivirus engineered MSCs in DSS colitis mice.

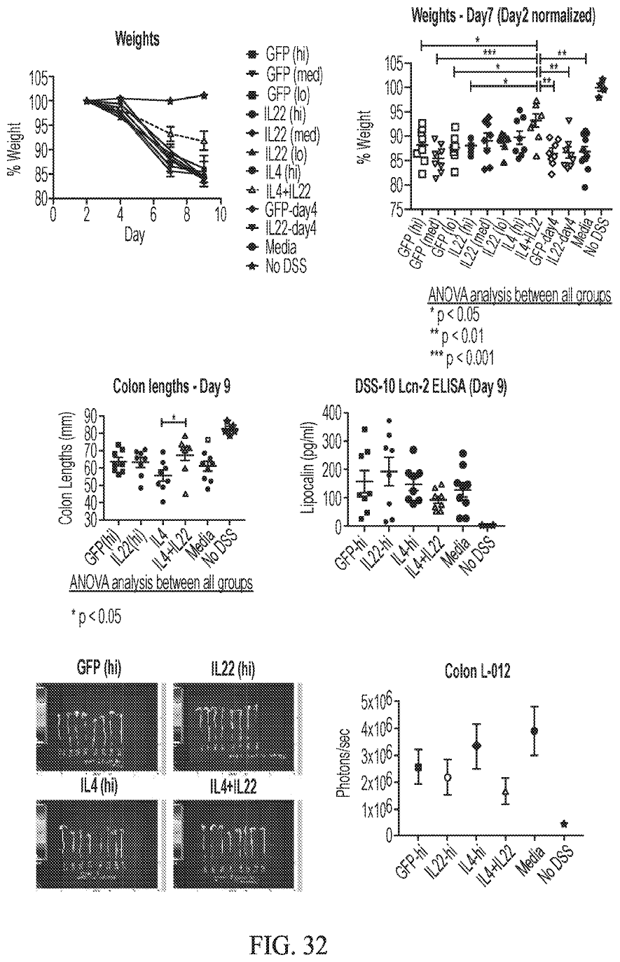

DSS colitis mice were then injected intraperitoneally with 4×10⁶ (hi), 1×10⁶ (med), or 0.25×10⁶ (lo) MSCs engineered by lentiviral transduction to express mouse IL-22, IL-4, or control GFP. Combination engineered mouse IL-4/IL-22 were injected with 4×10⁶ MSCs with equal parts MSC-IL-4 (2×10⁶) and MSC-IL-22 (2×10⁶). Colon lengths were measured on Day 9 upon sacrifice of mice. Stool protein was collected at Day 9 and lipocalin-2 (Lcn-2) levels measured by ELISA. In situ colon inflammation was measured on Day 9 after injection of L-012 and upon sacrifice of mice and dissection of colons. L-012 chemiluminescence was measured as photons per seconds. Injection cohorts and measurements were conducted in a double-blinded manner. Each cohort represents an average of 8-10 mice per group with error bars representing standard error of means (SEM). FIG. 32 shows improved weight, colon length, lipocalin-2 levels, and in situ colon inflammation L-012 levels from injected lentivirus engineered mouse IL-4/IL-22 combination MSCs in DSS colitis mice.

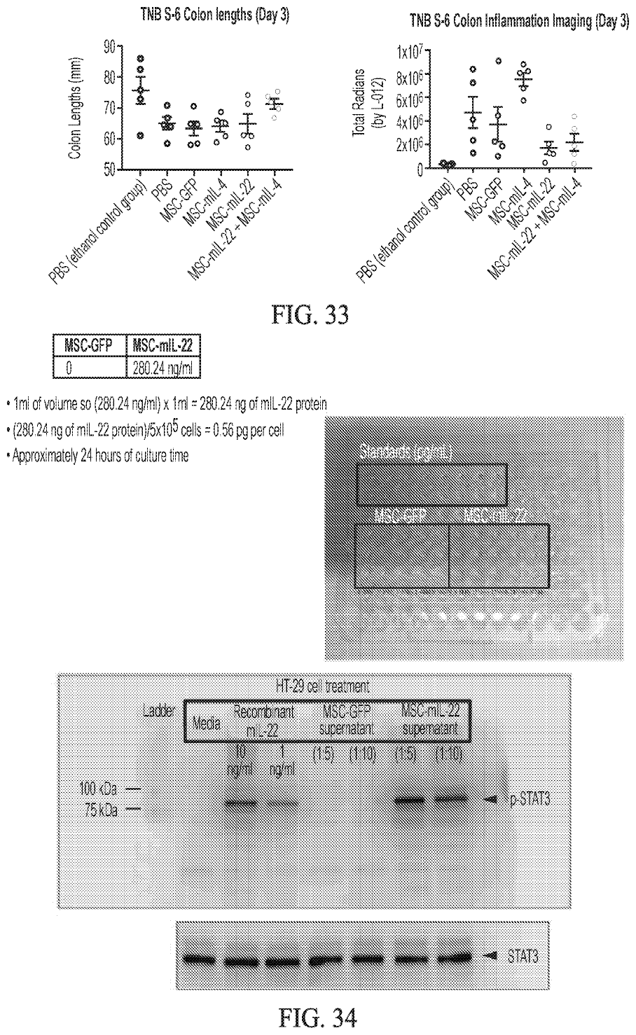

Colitis was then induced in another group of C57BL/6 mice by administering 2.5% 2,4,6-trinitrobenzene sulfonic acid (TNBS) in 50% ethanol via anal instillation on Day 0. 1×10⁶ MSCs engineered by lentiviral transduction to express mouse IL-22, IL-4, or control GFP were then administered via intraperitoneal injection. Combination engineered mouse IL-4/IL-22 were injected with 1×10⁶ MSCs with equal parts MSC-IL-4 (0.5×10⁶) and MSC-IL-22 (0.5×10⁶). Colon lengths were measured on Day 3 upon sacrifice of mice. In situ colon inflammation was measured on Day 3 after injection of L-012 and upon sacrifice of mice and dissection of colons. L-012 chemiluminescence was measured as photons per seconds. Injection cohorts and measurements were conducted in a double-blinded manner. Each cohort represents an average of 5 mice per group with error bars representing standard error of means (SEM). FIG. 33 shows improved colon length and in situ colon inflammation L-012 levels from injected lentivirus engineered mouse IL-22 and IL-4/IL-22 combination MSCs in TNBS colitis mice.

FIG. 34 shows secreted protein expression of mouse IL-22 as well as functional receptor signaling phospho-STAT3 activity of lentiviral transduced MSCs engineered to express mouse IL-22. 5×10⁵ lentiviral transduced mouse IL-22 or control GFP engineered MSCs were plated in 1 ml of culture media and supernatant collected 24 hours later and measured by ELISA. Supernatants were also added at indicated (1:5) or (1:10) dilution in 200 ul of culture media to 5×10⁵ HT-29 cells for 15 minutes and protein lysates made using M-PER lysis buffer with proteinase and phosphatase inhibitor cocktail. Protein lysates were run on denaturing SDS-PAGE gel and transferred to PVDF membrane and probed with antibody to phospho-STAT3 or total STAT3 in Western Blot chemiluminescence reactions.

### Example 8

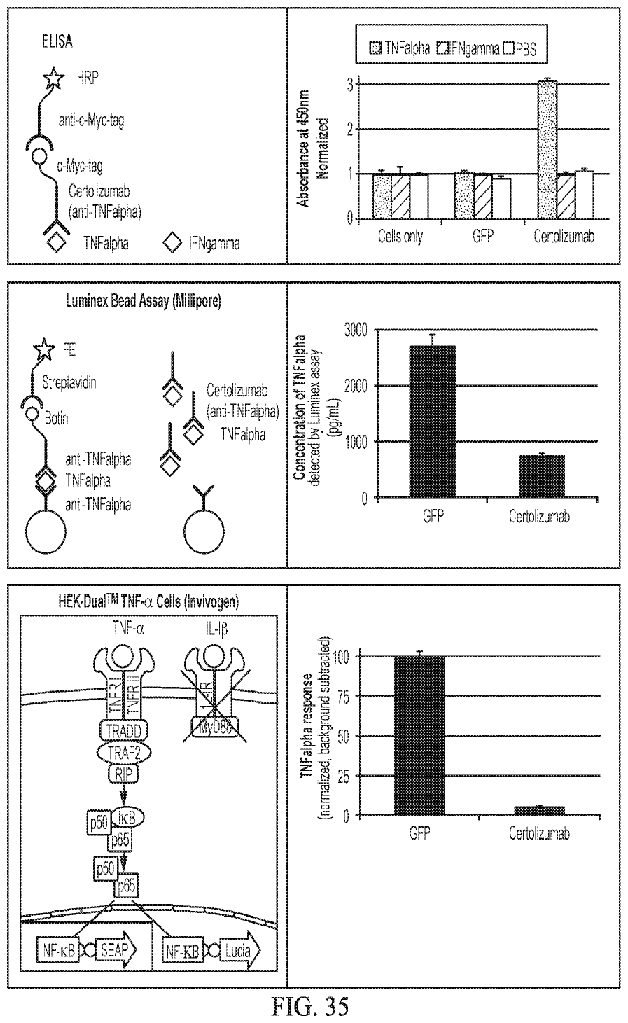

FIG. 35 shows the successful production, secretion, binding, and function antagonism of TNF-alpha by a TNF-alpha Fab antibody certolizumab produced by engineered MSCs. Top, supernatant from lentivirus transduced MSCs engineered to express c-Myc-tagged Certolizumab Fab antibody or control GFP was harvested after 24 hours. ELISA plates were coated with a 1 ng/ml of TNF-alpha, IFN-gamma, or PBS media and undiluted MSC supernatant incubated overnight followed by detection using anti-c-Myc antibody conjugated to horseradish peroxidase (HRP) and enzymatically detected using TMB substrate. Middle, engineered MSC supernatant was incubated with 1 ng/ml TNF-alpha for 1 hour then used to coat a Luminex plate overnight. Certolizumab competitively interfered with the binding to TNF-alpha from the Luminex capture/detection TNF-alpha capture antibody set and resulted in a lower detection signal from Luminex. Bottom, engineered MSC supernatant was incubated with 10 ng/ml TNF-alpha for 1 hour then added to 5×10⁴ TNF-alpha reporter cells (InvivoGen HEK-Dual TNF-alpha cells) that detects TNF-alpha and generates secreted embryonic alkaline phosphatase (SEAP). SEAP levels were then detected by QUANTI-BLUE®. All conditions were done as 3 biological replicates with error bars representing standard error of means (SEM).

### Example 9

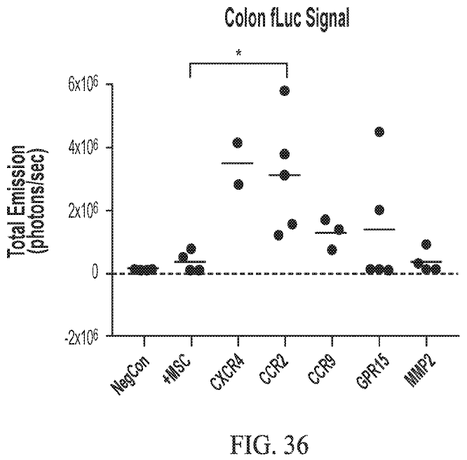

Next, MSCs were nucleofected with a firefly luciferase/GFP reporter plasmid (fLuc-GFP) and the indicated chemokine receptor plasmid in equal amounts (4 ug each). 2.5% 2,4,6-trinitrobenzene sulfonic acid (TNBS) in 50% ethanol was administered to C57BL/6 mice via anal instillation on Day 0 to induce colitis then 4×10⁵ engineered MSCs administered via intraperitoneal injection on Day 1. Mice were injected with D-luciferin and sacrificed on Day 2 (1 day after MSC injection), tissues dissected as indicated, and imaging performed on a Spectral Instruments Ami imager. Luciferase chemiluminescence was measured as photons per seconds. FIG. 36 shows tissue biodistribution and increased homing of MSCs to inflamed colon by engineered expression of chemokine receptors CXCR4, CCR2, CCR9, and GPR15 in TNBS colitis mice.

### Example 10

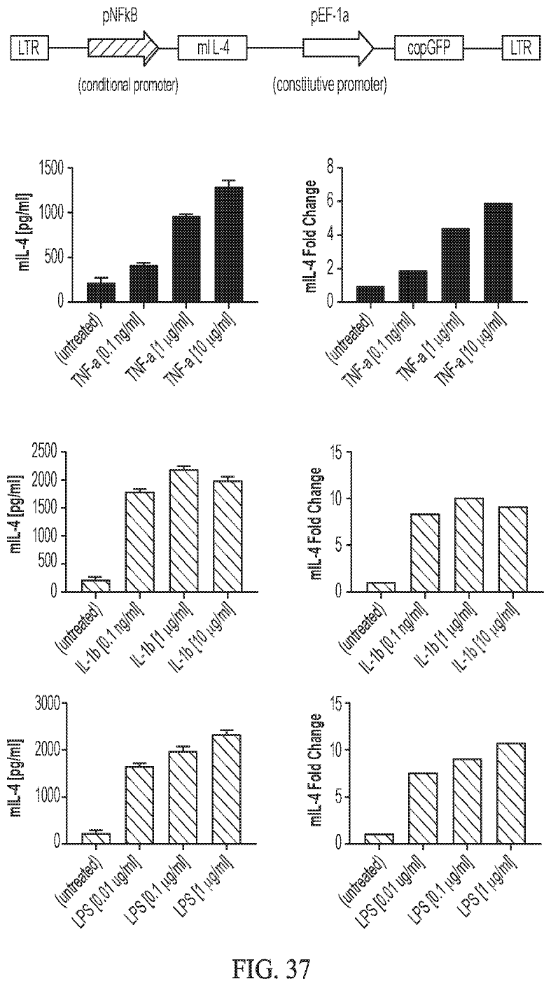

For this example, 5×10³ engineered MSCs received, by lentiviral transduction, a genetic circuit that included a conditional NF-kB (nuclear factor kappa-B) responsive promoter driving mouse IL-4 followed by a constitutive promoter driving GFP (FIG. 37, top). The transduced MSCs were then treated for 24 hours with the inflammatory cytokines TNF-alpha, IL-1beta, or lipopolysaccharide from E. coli (LPS) at concentrations of 0.1 ng/ml, 1 ng/ml, or 10 ng/ml in 200 al of culture media. Supernatant was collected 24 hours later and measured by ELISA. FIG. 37 shows a genetic circuit that delivered by lentiviral transduction into MSCs. This construct enabled the MSCs to sense inflammatory stimuli and respond via secretion of target payload IL-4. Left column shows measured concentration, right column shows fold-change from untreated condition. All conditions were done as three biological replicates with error bars representing standard error of means (SEM).

TABLE 2
Genetic elements and associated sequences

| Genetic | | | |
|---|---|---|---|
| Element | Name | DNA SEQUENCE | Protein Sequence |
| Promoter | CMV | GTTGACATTGATTATTGACTAGTTAT | |
| | | TAATAGTAATCAATTACGGGGTCAT | |
| | | TAGTTCATAGCCCATATATGGAGTTC | |
| | | CGCGTTACATAACTTACGGTAAATG | |
| | | GCCCGCCTGGCTGACCGCCCAACGA | |
| | | CCCCCGCCCATTGACGTCAATAATG | |
| | | ACGTATGTTCCCATAGTAACGCCAA | |
| | | TAGGGACTTTCCATTGACGTCAATG | |
| | | GGTGGAGTATTTACGGTAAACTGCC | |
| | | CACTTGGCAGTACATCAAGTGTATC | |
| | | ATATGCCAAGTACGCCCCCTATTGA | |
| | | CGTCAATGACGGTAAATGGCCCGCC | |
| | | TGGCATTATGCCCAGTACATGACCTT | |
| | | ATGGGACTTTCCTACTTGGCAGTACA | |
| | | TCTACGTATTAGTCATCGCTATTACC | |
| | | ATGGTGATGCGGTTTTGGCAGTACA | |
| | | TCAATGGGCGTGGATAGCGGTTTGA | |
| | | CTCACGGGGATTTCCAAGTCTCCACC | |
| | | CCATTGACGTCAATGGGAGTTTGTTT | |
| | | TGGCACCAAAATCAACGGGACTTTC | |
| | | CAAAATGTCGTAACAACTCCGCCCC | |
| | | ATTGACGCAAATGGGCGGTAGGCGT | |
| | | GTACGGTGGGAGGTCTATATAAGCA | |
| | | GAGCTC (SEQ ID NO: 1) | |
| | EF1a | GGCTCCGGTGCCCGTCAGTGGGCAG | |
| | | AGCGCACATCGCCCACAGTCCCCGA | |
| | | GAAGTTGGGGGGAGGGGTCGGCAA | |
| | | TTGAACCGGTGCCTAGAGAAGGTGG | |
| | | CGCGGGGTAAACTGGGAAAGTGATG | |
| | | CCGTGTACTGGCTCCGCCTTTTTCCC | |
| | | GAGGGTGGGGGAGAACCGTATATA | |
| | | AGTGCAGTAGTCGCCGTGAACGTTC | |
| | | TTTTTCGCAACGGGTTTGCCGCCAG | |
| | | AACACAGGTAAGTGCCGTGTGTGGT | |
| | | TCCCGCGGGCCTGGCCTCTTTACGG | |
| | | GTTATGGCCCTTGCGTGCCTTGAATT | |
| | | ACTTCCACCTGGCTGCAGTACGTGA | |
| | | TTCTTGATCCCGAGCTTCGGGTTGGA | |
| | | AGTGGGTGGGAGAGTTCGAGGCCTT | |
| | | GCGCTTAAGGAGCCCCTTCGCCTCG | |
| | | TGCTTGAGTTGAGGCCTGGCCTGGG | |
| | | CGCTGGGGCCGCCGCGTGCGAATCT | |
| | | GGTGGCACCTTCGCGCCTGTCTCGCT | |
| | | GCTTTCGATAAGTCTCTAGCCATTTA | |
| | | AAATTTTTGATGACCTGCTGCGACG | |
| | | CTTTTTTTCTGGCAAGATAGTCTTGT | |
| | | AAATGCGGGCCAAGATCTGCACACT | |
| | | GGTATTTCGGTTTTTGGGGCCGCGG | |
| | | GCGGCGACGGGGCCCGTGCGTCCCA | |
| | | GCGCACATGTTCGGCGAGGCGGGGC | |
| | | CTGCGAGCGCGACCACCGAGAATCG | |
| | | GACGGGGGTAGTCTCAAGCTGGCCG | |
| | | GCCTGCTCTGGTGCCTGTCCTCGCGC | |
| | | CGCCGTGTATCGCCCCGCCCCGGGC | |
| | | GGCAAGGCTGGCCCGGTCGGCACCA | |
| | | GTTGCGTGAGCGGAAAGATGGCCGC | |
| | | TTCCCGGTCCTGCTGCAGGGAGCTC | |
| | | AAAATGGAGGACGCGGCGCTCGGG | |
| | | AGAGCGGGCGGGTGAGTCACCCACA | |
| | | CAAAGGAAAAGGGCCTTTCCGTCCT | |
| | | CAGCCGTCGCTTCATGTGACTCCAC | |
| | | GGAGTACCGGGCGCCGTCCAGGCAC | |
| | | CTCGATTAGTTCTCGAGCTTTTGGAG | |
| | | TACGTCGTCTTTAGGTTGGGGGGAG | |
| | | GGGTTTTATGCGATGGAGTTTCCCC | |
| | | ACACTGAGTGGGTGGAGACTGAAGT | |
| | | TAGGCCAGCTTGGCACTTGATGTAA | |
| | | TTCTCCTTGGAATTTGCCCTTTTTGA | |
| | | GTTTGGATCTTGGTTCATTCTCAAGC | |
| | | CTCAGACAGTGGTTCAAAGTTTTTTT | |
| | | CTTCCATTTCAGGTGTCGTGA (SEQ | |
| | | ID NO: 2) | |
| | EFS | GGATCTGCGATCGCTCCGGTGCCCG | |
| | | TCAGTGGGCAGAGCGCACATCGCCC | |
| | | ACAGTCCCCGAGAAGTTGGGGGGAG | |
| | | GGGTCGGCAATTGAACCGGTGCCTA | |
| | | GAGAAGGTGGCGCGGGGTAAACTG | |
| | | GGAAAGTGATGTCGTGTACTGGCTC | |
| | | CGCCTTTTTCCCGAGGGTGGGGGAG | |
| | | AACCGTATATAAGTGCAGTAGTCGC | |
| | | CGTGAACGTTCTTTTTCGCAACGGGT | |
| | | TTGCCGCCAGAACACAGCTGAAGCT | |
| | | TCGAGGGGCTCGCATCTCTCCTTCAC | |
| | | GCGCCCGCCGCCCTACCTGAGGCCG | |
| | | CCATCCACGCCGGTTGAGTCGCGTT | |
| | | CTGCCGCCTCCCGCCTGTGGTGCCTC | |
| | | CTGAACTGCGTCCGCCGTCTAGGTA | |
| | | AGTTTAAAGCTCAGGTCGAGACCGG | |
| | | GCCTTTGTCCGGCGCTCCCTTGGAGC | |
| | | CTACCTAGACTCAGCCGGCTCTCCA | |
| | | CGCTTTGCCTGACCCTGCTTGCTCAA | |
| | | CTCTACGTCTTTGTTTCGTTTTCTGTT | |
| | | CTGCGCCGTTACAGATCCAAGCTGT | |
| | | GACCGGCGCCTAC (SEQ ID NO: 3) | |
| | MND | TTTATTTAGTCTCCAGAAAAAGGGG | |
| | | GGAATGAAAGACCCCACCTGTAGGT | |
| | | TTGGCAAGCTAGGATCAAGGTTAGG | |
| | | AACAGAGAGACAGCAGAATATGGG | |
| | | CCAAACAGGATATCTGTGGTAAGCA | |
| | | GTTCCTGCCCCGGCTCAGGGCCAAG | |
| | | AACAGTTGGAACAGCAGAATATGGG | |
| | | CCAAACAGGATATCTGTGGTAAGCA | |
| | | GTTCCTGCCCCGGCTCAGGGCCAAG | |
| | | AACAGATGGTCCCCAGATGCGGTCC | |
| | | CGCCCTCAGCAGTTTCTAGAGAACC | |
| | | ATCAGATGTTTCCAGGGTGCCCCAA | |
| | | GGACCTGAAATGACCCTGTGCCTTA | |
| | | TTTGAACTAACCAATCAGTTCGCTTC | |
| | | TCGCTTCTGTTCGCGCGCTTCTGCTC | |
| | | CCCGAGCTCAATAAAAGAGCCCA | |
| | | (SEQ ID NO: 4) | |
| | PGK | GGGGTTGGGGTTGCGCCTTTTCCAA | |
| | | GGCAGCCCTGGGTTTGCGCAGGGAC | |
| | | GCGGCTGCTCTGGGCGTGGTTCCGG | |
| | | GAAACGCAGCGGCGCCGACCCTGGG | |
| | | TCTCGCACATTCTTCACGTCCGTTCG | |
| | | CAGCGTCACCCGGATCTTCGCCGCT | |
| | | ACCCTTGTGGGCCCCCCGGCGACGC | |
| | | TTCCTGCTCCGCCCCTAAGTCGGGA | |
| | | AGGTTCCTTGCGGTTCGCGGCGTGC | |
| | | CGGACGTGACAAACGGAAGCCGCA | |
| | | CGTCTCACTAGTACCCTCGCAGACG | |
| | | GACAGCGCCAGGGAGCAATGGCAG | |
| | | CGCGCCGACCGCGATGGGCTGTGGC | |
| | | CAATAGCGGCTGCTCAGCGGGGCGC | |
| | | GCCGAGAGCAGCGGCCGGGAAGGG | |
| | | GCGGTGCGGGAGGCGGGGTGTGGG | |
| | | GCGGTAGTGTGGGCCCTGTTCCTGC | |
| | | CCGCGCGGTGTTCCGCATTCTGCAA | |
| | | GCCTCCGGAGCGCACGTCGGCAGTC | |
| | | GGCTCCCTCGTTGACCGAATCACCG | |
| | | ACCTCTCTCCCCAG (SEQ ID NO: 5) | |
| | SFFV | GTAACGCCATTTTGCAAGGCATGGA | |
| | | AAAATACCAAACCAAGAATAGAGA | |
| | | AGTTCAGATCAAGGGCGGGTACATG | |
| | | AAAATAGCTAACGTTGGGCCAAACA | |
| | | GGATATCTGCGGTGAGCAGTTTCGG | |
| | | CCCCGGCCCGGGGCCAAGAACAGAT | |
| | | GGTCACCGCAGTTTCGGCCCCGGCC | |
| | | CGAGGCCAAGAACAGATGGTCCCCA | |
| | | GATATGGCCCAACCCTCAGCAGTTT | |
| | | CTTAAGACCCATCAGATGTTTCCAG | |
| | | GCTCCCCCAAGGACCTGAAATGACC | |
| | | CTGCGCCTTATTTGAATTAACCAATC | |
| | | AGCCTGCTTCTCGCTTCTGTTCGCGC | |
| | | GCTTCTGCTTCCCGAGCTCTATAAAA | |
| | | GAGCTCACAACCCCTCACTCGGCGC | |
| | | GCCAGTCCTCCGACAGACTGAGTCG | |
| | | CCCGGG (SEQ ID NO: 6) | |
| | SV40 | CTGTGGAATGTGTGTCAGTTAGGGT | |
| | | GTGGAAAGTCCCCAGGCTCCCCAGC | |
| | | AGGCAGAAGTATGCAAAGCATGCAT | |
| | | CTCAATTAGTCAGCAACCAGGTGTG | |
| | | GAAAGTCCCCAGGCTCCCCAGCAGG | |
| | | CAGAAGTATGCAAAGCATGCATCTC | |
| | | AATTAGTCAGCAACCATAGTCCCGC | |
| | | CCCTAACTCCGCCCATCCCGCCCCTA | |
| | | ACTCCGCCCAGTTCCGCCCATTCTCC | |
| | | GCCCCATGGCTGACTAATTTTTTTTA | |
| | | TTTATGCAGAGGCCGAGGCCGCCTC | |
| | | TGCCTCTGAGCTATTCCAGAAGTAG | |
| | | TGAGGAGGCTTTTTTGGAGGCCTAG | |
| | | GCTTTTGCAAAAAGCT (SEQ ID NO: | |
| | | 7) | |
| | UbC | GCGCCGGGTTTTGGCGCCTCCCGCG | |
| | | GGCGCCCCCCTCCTCACGGCGAGCG | |
| | | CTGCCACGTCAGACGAAGGGCGCAG | |
| | | GAGCGTTCCTGATCCTTCCGCCCGG | |
| | | ACGCTCAGGACAGCGGCCCGCTGCT | |
| | | CATAAGACTCGGCCTTAGAACCCCA | |
| | | GTATCAGCAGAAGGACATTTTAGGA | |
| | | CGGGACTTGGGTGACTCTAGGGCAC | |
| | | TGGTTTTCTTTCCAGAGAGCGGAAC | |
| | | AGGCGAGGAAAAGTAGTCCCTTCTC | |
| | | GGCGATTCTGCGGAGGGATCTCCGT | |
| | | GGGGCGGTGAACGCCGATGATTATA | |
| | | TAAGGACGCGCCGGGTGTGGCACAG | |
| | | CTAGTTCCGTCGCAGCCGGGATTTG | |
| | | GGTCGCGGTTCTTGTTTGTGGATCGC | |
| | | TGTGATCGTCACTTGGTGAGTTGCG | |
| | | GGCTGCTGGGCTGGCCGGGGCTTTC | |
| | | GTGGCCGCCGGGCCGCTCGGTGGGA | |
| | | CGGAAGCGTGTGGAGAGACCGCCA | |
| | | AGGGCTGTAGTCTGGGTCCGCGAGC | |
| | | AAGGTTGCCCTGAACTGGGGGTTGG | |
| | | GGGGAGCGCACAAAATGGCGGCTGT | |
| | | TCCCGAGTCTTGAATGGAAGACGCT | |
| | | TGTAAGGCGGGCTGTGAGGTCGTTG | |
| | | AAACAAGGTGGGGGGCATGGTGGG | |
| | | CGGCAAGAACCCAAGGTCTTGAGGC | |
| | | CTTCGCTAATGCGGGAAAGCTCTTA | |
| | | TTCGGGTGAGATGGGCTGGGGCACC | |
| | | ATCTGGGGACCCTGACGTGAAGTTT | |
| | | GTCACTGACTGGAGAACTCGGGTTT | |
| | | GTCGTCTGGTTGCGGGGGCGGCAGT | |
| | | TATGCGGTGCCGTTGGGCAGTGCAC | |
| | | CCGTACCTTTGGGAGCGCGCGCCTC | |
| | | GTCGTGTCGTGACGTCACCCGTTCTG | |
| | | TTGGCTTATAATGCAGGGTGGGGCC | |
| | | ACCTGCCGGTAGGTGTGCGGTAGGC | |
| | | TTTTCTCCGTCGCAGGACGCAGGGT | |
| | | TCGGGCCTAGGGTAGGCTCTCCTGA | |
| | | ATCGACAGGCGCCGGACCTCTGGTG | |
| | | AGGGGAGGGATAAGTGAGGCGTCA | |
| | | GTTTCTTTGGTCGGTTTTATGTACCT | |
| | | ATCTTCTTAAGTAGCTGAAGCTCCG | |
| | | GTTTTGAACTATGCGCTCGGGGTTG | |
| | | GCGAGTGTGTTTTGTGAAGTTTTTTA | |
| | | GGCACCTTTTGAAATGTAATCATTTG | |
| | | GGTCAATATGTAATTTTCAGTGTTAG | |
| | | ACTAGTAAAGCTTCTGCAGGTCGAC | |
| | | TCTAGAAAATTGTCCGCTAAATTCT | |
| | | GGCCGTTTTTGGCTTTTTTGTTAGAC | |
| | | (SEQ ID NO: 8) | |
| Effector | PD-L1 | ATGAGAATTTTTGCCGTGTTTATTTT | MRIFAVFIFMTYWHLLNAF |
| | (B7H1) | TATGACTTACTGGCACCTTCTTAACG | TVTVPKDLYVVEYGSNMTI |
| | | CTTTCACGGTTACTGTTCCGAAGGAT | ECKFPVEKQLDLAALIVYW |
| | | CTGTACGTTGTAGAATACGGTAGCA | EMEDKNIIQFVHGEEDLKV |
| | | ACATGACTATAGAGTGCAAATTTCC | QHSSYRQRARLLKDQLSLG |
| | | CGTTGAGAAACAACTTGATCTTGCC | NAALQITDVKLQDAGVYRC |
| | | GCCTTGATCGTCTACTGGGAAATGG | MISYGGADYKRITVKVNAP |
| | | AGGACAAAAATATAATACAGTTCGT | YNKINQRILVVDPVTSEHEL |
| | | TCATGGAGAGGAGGACCTTAAAGTA | TCQAEGYPKAEVIWTSSDH |
| | | CAGCACTCTTCATACAGACAGCGCG | QVLSGKTTTTNSKREEKLF |
| | | CGCGGCTTTTGAAAGATCAGCTTTCT | NVTSTLRINTTTNEIFYCTFR |
| | | CTGGGCAACGCGGCTCTTCAAATTA | RLDPEENHTAELVIPELPLA |
| | | CAGATGTCAAATTGCAAGATGCTGG | HPPNERTHLVILGAILLCLG |
| | | AGTATACAGATGTATGATCTCTTAC | VALTFIFRLRKGRMMDVKK |
| | | GGTGGCGCAGATTATAAGAGGATTA | CGIQDTNSKKQSDTHLEET |
| | | CGGTAAAAGTAAACGCCCCCTATAA | (SEQ ID NO: 21) |
| | | CAAAATCAATCAGAGGATTCTGGTC | |
| | | GTCGACCCGGTAACGAGTGAGCACG | |
| | | AATTGACTTGCCAAGCTGAAGGCTA | |
| | | CCCGAAGGCGGAGGTCATATGGACT | |
| | | TCCTCTGATCATCAAGTTTTGTCTGG | |
| | | CAAAACGACAACTACCAACAGTAAG | |
| | | AGAGAGGAAAAGTTGTTCAACGTTA | |
| | | CGTCCACACTCAGAATAAACACGAC | |
| | | CACTAACGAGATTTTTTACTGCACGT | |
| | | TTCGACGACTTGACCCGGAAGAAAA | |
| | | TCACACAGCAGAGCTTGTGATCCCT | |
| | | GAACTGCCCCTGGCCCATCCACCAA | |
| | | ATGAACGAACTCATCTGGTCATTCT | |
| | | CGGTGCTATTTTGTTGTGTCTCGGAG | |
| | | TGGCACTTACCTTTATATTTAGACTC | |
| | | CGAAAAGGTCGCATGATGGACGTCA | |
| | | AAAAGTGCGGAATCCAAGACACCA | |
| | | ACAGTAAAAAACAATCCGATACTCA | |
| | | TCTTGAAGAAACA (SEQ ID NO: 9) | |
| | IL-10 | ATGCATTCTAGCGCGTTGCTGTGTTG | MHSSALLCCLVLLTGVRAS |
| | | CCTCGTGCTGCTCACTGGGGTTCGG | PGQGTQSENSCTHFPGNLP |
| | | GCCTCCCCTGGTCAAGGAACCCAAT | NMLRDLRDAFSRVKTFFQM |
| | | CAGAGAACTCATGCACGCATTTTCC | KDQLDNLLLKESLLEDFKG |
| | | GGGGAACTTGCCGAATATGTTGCGA | YLGCQALSEMIQFYLEEVM |
| | | GACCTGCGCGATGCATTTTCCAGAG | PQAENQDPDIKAHVNSLGE |
| | | TAAAGACCTTTTTCCAAATGAAGGA | NLKTLRLRLRRCHRFLPCE |
| | | CCAGCTCGATAATTTGTTGCTCAAA | NKSKAVEQVKNAFNKLQE |
| | | GAGAGTTTGCTGGAGGACTTTAAAG | KGIYKAMSEFDIFINYIEAY |
| | | GTTACCTCGGATGCCAGGCTCTGTCT | MTMKIRN (SEQ ID NO: 22) |
| | | GAGATGATTCAATTTTATTTGGAGG | |
| | | AAGTAATGCCGCAGGCGGAAAACC | |
| | | AGGACCCCGATATAAAGGCTCATGT | |
| | | AAACTCTCTGGGTGAAAACCTTAAA | |
| | | ACACTGAGATTGCGCCTCCGAAGAT | |
| | | GTCATAGGTTCCTTCCGTGCGAAAA | |
| | | TAAGAGCAAGGCTGTTGAACAGGTG | |
| | | AAAAATGCTTTTAACAAACTTCAAG | |
| | | AGAAAGGGATTTATAAGGCAATGTC | |
| | | AGAGTTTGACATTTTCATCAACTAC | |
| | | ATAGAAGCGTACATGACGATGAAAA | |
| | | TTCGCAAT (SEQ ID NO: 10) | |
| | IL-11 | ATGAACTGCGTCTGCCGGTTGGTAT | MNCVCRLVLVVLSLWPDT |
| | | TGGTAGTCTTGTCTTTGTGGCCGGAT | AVAPGPPPGPPRVSPDPRAE |
| | | ACTGCCGTCGCTCCCGGCCCCCCGC | LDSTVLLTRSLLADTRQLA |
| | | CTGGTCCTCCCCGAGTCTCACCAGA | AQLRDKFPADGDHNLDSLP |
| | | CCCGAGAGCAGAACTCGATTCTACG | TLAMSAGALGALQLPGVLT |
| | | GTTCTGTTGACCCGGTCACTGCTGGC | RLRADLLSYLRHVQWLRR |
| | | GGACACCCGGCAACTCGCCGCCCAA | AGGSSLKTLEPELGTLQARL |
| | | CTGCGGGACAAATTTCCTGCAGACG | DRLLRRLQLLMSRLALPQP |
| | | GCGATCACAACTTGGACTCTCTTCC | PPDPPAPPLAPPSSAWGGIR |
| | | AACACTTGCAATGTCAGCTGGCGCC | AAHAILGGLHLTLDWAVR |
| | | CTTGGTGCTCTGCAATTGCCGGGGG | GLLLLKTRL (SEQ ID NO: |
| | | TCCTGACGAGACTGCGAGCGGATCT | 23) |
| | | GCTCAGCTACCTGAGGCACGTTCAA | |
| | | TGGCTGAGACGGGCGGGAGGAAGC | |
| | | TCACTTAAGACACTGGAGCCCGAGC | |
| | | TCGGCACCTTGCAAGCTCGGCTGGA | |
| | | TCGGCTCCTGAGACGATTGCAACTT | |
| | | CTCATGTCTCGACTGGCACTTCCACA | |
| | | ACCACCCCCTGATCCCCCCGCGCCC | |
| | | CCACTGGCCCCCCCGTCATCTGCGT | |
| | | GGGGAGGCATCAGAGCAGCCCATGC | |
| | | TATTTTGGGGGGACTCCATCTCACCC | |
| | | TTGATTGGGCGGTGCGGGGCCTCCT | |
| | | TCTCTTGAAAACGCGGCTT (SEQ ID | |
| | | NO: 11) | |
| | IL-13 | ATGCATCCCCTGCTCAATCCCCTCTT | MHPLLNPLLLALGLMALLL |
| | | GCTGGCGCTTGGCCTCATGGCTCTG | TTVIALTCLGGFASPGPVPP |
| | | CTCCTGACGACTGTCATAGCTCTTAC | STALRELIEELVNITQNQKA |
| | | ATGCCTGGGTGGTTTCGCAAGCCCT | PLCNGSMVWSINLTAGMY |
| | | GGGCCAGTCCCGCCGTCAACAGCAC | CAALESLINVSGCSAIEKTQ |
| | | TTAGAGAGCTCATAGAAGAACTCGT | RMLSGFCPHKVSAGQFSSL |
| | | CAACATCACGCAGAACCAAAAAGCC | HVRDTKIEVAQFVKDLLLH |
| | | CCGTTGTGCAACGGTAGCATGGTAT | LKKLFREGRFN (SEQ ID |
| | | GGTCAATCAACCTGACAGCAGGGAT | NO: 24) |
| | | GTATTGTGCCGCTTTGGAGTCCTTGA | |
| | | TTAATGTTTCCGGTTGCAGTGCAATT | |
| | | GAGAAAACACAGCGAATGCTGTCTG | |
| | | GCTTCTGTCCTCACAAAGTTAGCGC | |
| | | AGGGCAATTTAGTTCCCTCCATGTA | |
| | | AGGGACACTAAAATAGAGGTCGCTC | |
| | | AATTCGTTAAGGATTTGCTTCTTCAT | |
| | | TTGAAGAAGCTGTTCAGGGAGGGCA | |
| | | GGTTTAAT (SEQ ID NO: 12) | |
| | IL-4 | ATGGGGCTCACCTCACAGCTCCTGC | MGLTSQLLPPLFFLLACAG |
| | | CGCCGCTCTTTTTCCTTCTCGCCTGC | NFVHGHKCDITLQEIIKTLN |
| | | GCGGGTAATTTTGTTCACGGTCATA | SLTEQKTLCTELTVTDIFAA |
| | | AGTGTGATATAACTCTGCAGGAGAT | SKNTTEKETFCRAATVLRQ |
| | | AATCAAGACTCTTAATTCTCTCACA | FYSHHEKDTRCLGATAQQF |
| | | GAGCAGAAAACACTTTGCACTGAGC | HRHKQLIRFLKRLDRNLWG |
| | | TGACGGTCACCGACATCTTCGCTGC | LAGLNSCPVKEANQSTLEN |
| | | ATCTAAGAATACCACCGAAAAGGAA | FLERLKTIMREKYSKCSS |
| | | ACATTTTGCCGGGCTGCGACAGTTTT | (SEQ ID NO: 25) |
| | | GCGGCAGTTTTACTCCCACCATGAG | |
| | | AAAGACACGCGATGCCTTGGTGCCA | |
| | | CAGCTCAGCAATTCCATAGGCATAA | |
| | | ACAATTGATTCGATTTCTTAAGCGG | |
| | | CTTGATCGAAACCTGTGGGGGCTTG | |
| | | CGGGGTTGAACTCATGCCCGGTTAA | |
| | | AGAAGCAAATCAGTCTACTCTGGAG | |
| | | AATTTTTTGGAACGGCTTAAGACGA | |
| | | TTATGAGAGAAAAATACTCCAAATG | |
| | | TTCCTCC (SEQ ID NO: 13) | |
| | IL-35 | ATGACGCCGCAACTTTTGCTGGCAC | MTPQLLLALVLWASCPPCS |
| | fusion | TTGTGTTGTGGGCTTCTTGTCCACCT | GRKGPPAALTLPRVQCRAS |
| | | TGCTCAGGGCGCAAAGGGCCTCCGG | RYPIAVDCSWTLPPAPNSTS |
| | | CTGCTTTGACGTTGCCAAGAGTGCA | PVSFIATYRLGMAARGHSW |
| | | GTGCCGGGCCTCCCGATACCCTATA | PCLQQTPTSTSCTITDVQLFS |
| | | GCTGTGGACTGTTCTTGGACATTGCC | MAPYVLNVTAVHPWGSSSS |
| | | GCCGGCCCCGAACTCCACCTCACCC | FVPFITEHIIKPDPPEGVRLSP |
| | | GTCTCCTTCATTGCCACTTACCGACT | LAERQLQVQWEPPGSWPFP |
| | | GGGAATGGCAGCCAGGGGACACAG | EIFSLKYWIRYKRQGAARF |
| | | TTGGCCATGCCTGCAACAGACACCT | HRVGPIEATSFILRAVRPRA |
| | | ACTTCAACCAGCTGTACGATCACAG | RYYVQVAAQDLTDYGELS |
| | | ACGTCCAACTTTTCAGCATGGCACC | DWSLPATATMSLGKGGGS |
| | | ATACGTTCTTAACGTAACTGCAGTA | GGGSGGGSGGGSRNLPVAT |
| | | CATCCGTGGGGGAGTTCTAGTAGCT | PDPGMFPCLHHSQNLLRAV |
| | | TCGTTCCGTTCATAACTGAGCACATC | SNMLQKARQTLEFYPCTSE |
| | | ATAAAACCAGACCCACCTGAGGGAG | EIDHEDITKDKTSTVEACLP |
| | | TCCGCTTGTCTCCTCTTGCCGAGAGG | LELTKNESCLNSRETSFITN |
| | | CAACTTCAAGTTCAGTGGGAACCGC | GSCLASRKTSFMMALCLSSI |
| | | CGGGGTCTTGGCCGTTTCCCGAAAT | YEDLKMYQVEFKTMNAKL |
| | | ATTTTCACTTAAATACTGGATTAGAT | LMDPKRQIFLDQNMLAVID |
| | | ATAAAAGGCAAGGTGCGGCGAGATT | ELMQALNFNSETVPQKSSL |
| | | CCATCGGGTCGGGCCAATAGAAGCT | EEPDFYKTKIKLCILLHAFRI |
| | | ACGAGTTTTATCCTCCGAGCAGTTC | RAVTIDRVMSYLNAS (SEQ |
| | | GGCCGCGGGCACGATATTATGTGCA | ID NO: 26) |
| | | AGTTGCGGCACAGGATCTTACTGAC | |
| | | TACGGCGAACTCAGCGACTGGAGTC | |
| | | TGCCTGCAACTGCGACCATGTCACT | |
| | | GGGAAAGGGAGGAGGGAGTGGTGG | |
| | | CGGCAGCGGCGGAGGCAGTGGCGG | |
| | | CGGCAGCCGCAATCTGCCTGTCGCA | |
| | | ACTCCAGATCCGGGGATGTTCCCGT | |
| | | GTCTGCATCATAGCCAAAATCTGCT | |
| | | TAGGGCCGTCTCAAATATGCTCCAA | |
| | | AAAGCGAGACAGACGCTTGAATTTT | |
| | | ATCCGTGCACAAGTGAAGAGATTGA | |
| | | CCATGAGGACATCACGAAGGACAA | |
| | | AACGAGTACAGTGGAAGCCTGTCTG | |
| | | CCTCTTGAACTCACTAAAAACGAAA | |
| | | GCTGCCTGAATAGTCGAGAAACTTC | |
| | | ATTTATAACCAACGGCTCATGTCTTG | |
| | | CGAGCCGAAAAACAAGTTTTATGAT | |
| | | GGCTTTGTGTCTCTCTAGTATTTATG | |
| | | AGGATCTGAAAATGTACCAGGTTGA | |
| | | GTTCAAGACAATGAACGCTAAACTC | |
| | | CTTATGGACCCGAAACGGCAGATCT | |
| | | TTCTCGATCAAAACATGCTGGCTGTT | |
| | | ATCGACGAGCTCATGCAGGCACTGA | |
| | | ATTTTAATAGCGAGACCGTCCCGCA | |
| | | AAAAAGCTCCTTGGAGGAGCCGGAC | |
| | | TTTTATAAGACGAAGATCAAACTGT | |
| | | GCATCCTCCTCCACGCATTTCGCATA | |
| | | CGAGCGGTTACCATTGACCGGGTAA | |
| | | TGTCCTATCTGAATGCAAGT (SEQ ID | |
| | | NO: 14) | |
| | IL-22 | ATGGCTGCGCTCCAAAAAAGTGTGA | MAALQKSVSSFLMGTLATS |
| | | GTTCCTTTTTGATGGGCACGCTCGCA | CLLLLALLVQGGAAAPISSH |
| | | ACTAGCTGCTTGCTTCTGCTGGCGTT | CRLDKSNFQQPYITNRTFM |
| | | GCTCGTACAGGGTGGTGCTGCCGCA | LAKEASLADNNTDVRLIGE |
| | | CCAATATCATCCCATTGCCGCCTCG | KLFHGVSMSERCYLMKQV |
| | | ACAAAAGTAACTTTCAGCAGCCGTA | LNFTLEEVLFPQSDRFQPYM |
| | | CATAACTAACCGCACCTTCATGCTC | QEVVPFLARLSNRLSTCHIE |
| | | GCGAAAGAAGCGAGCCTCGCTGACA | GDDLHIQRNVQKLKDTVK |
| | | ACAACACGGATGTCCGATTGATTGG | KLGESGEIKAIGELDLLFMS |
| | | CGAAAAACTGTTTCATGGAGTTTCC | LRNACI (SEQ ID NO: 27) |
| | | ATGAGTGAACGATGTTATTTGATGA | |
| | | AACAAGTACTTAACTTCACATTGGA | |
| | | AGAAGTTCTCTTCCCACAGAGTGAT | |
| | | CGGTTCCAACCTTATATGCAGGAGG | |
| | | TTGTCCCTTTTTTGGCCCGACTGTCT | |
| | | AATAGGCTTTCAACGTGCCACATTG | |
| | | AAGGCGATGACCTTCACATACAAAG | |
| | | GAATGTGCAGAAACTGAAAGATACT | |
| | | GTAAAAAAACTTGGAGAGTCAGGA | |
| | | GAAATCAAAGCCATAGGGGAGCTCG | |
| | | ATCTTCTTTTCATGTCACTGCGGAAC | |
| | | GCCTGTATT (SEQ ID NO: 15) | |
| | TSG-6 | ATGATAATACTGATTTATTTGTTCTT | MIILIYLFLLLWEDTQGWGF |
| | | GCTCCTGTGGGAGGACACGCAGGGA | KDGIFHNSIWLERAAGVYH |
| | | TGGGGCTTTAAGGACGGTATATTTC | REARSGKYKLTYAEAKAV |
| | | ACAATAGTATATGGCTCGAACGAGC | CEFEGGHLATYKQLEAARK |
| | | GGCAGGCGTTTACCATAGAGAAGCA | IGFHVCAAGWMAKGRVGY |
| | | CGATCTGGAAAATATAAGTTGACAT | PIVKPGPNCGFGKTGIIDYGI |
| | | ACGCAGAGGCGAAAGCTGTATGTGA | RLNRSERWDAYCYNPHAK |
| | | GTTCGAAGGGGGACATCTTGCAACC | ECGGVFTDPKQIFKSPGFPN |
| | | TATAAACAATTGGAGGCTGCGAGAA | EYEDNQICYWHIRLKYGQR |
| | | AGATCGGATTCCACGTCTGCGCTGC | IHLSFLDFDLEDDPGCLADY |
| | | TGGGTGGATGGCCAAAGGCAGGGTA | VEIYDSYDDVHGFVGRYCG |
| | | GGTTACCCTATAGTCAAGCCTGGTC | DELPDDIISTGNVMTLKFLS |
| | | CTAACTGTGGTTTTGGTAAGACAGG | DASVTAGGFQIKYVAMDPV |
| | | GATTATCGACTACGGTATAAGGCTC | SKSSQGKNTSTTSTGNKNFL |
| | | AATCGAAGCGAGAGATGGGATGCCT | AGRFSHL(SEQ ID NO: 28) |
| | | ATTGCTATAATCCCCACGCGAAAGA | |
| | | ATGCGGCGGTGTCTTTACGGACCCA | |
| | | AAGCAGATCTTTAAGAGCCCAGGTT | |
| | | TTCCAAACGAGTACGAGGATAACCA | |
| | | AATATGTTATTGGCACATTAGATTG | |
| | | AAATATGGGCAGAGAATACACCTTA | |
| | | GTTTTCTCGATTTCGATCTGGAGGAT | |
| | | GATCCAGGGTGTCTGGCGGATTATG | |
| | | TTGAGATCTATGATAGCTACGATGA | |
| | | CGTTCACGGTTTCGTTGGGAGATAC | |
| | | TGCGGGGACGAACTCCCCGACGACA | |
| | | TCATAAGTACTGGTAATGTAATGAC | |
| | | TCTCAAATTTCTGAGCGATGCTTCAG | |
| | | TGACCGCAGGCGGATTCCAAATTAA | |
| | | GTATGTGGCAATGGACCCCGTATCC | |
| | | AAAAGCAGCCAGGGAAAAAATACC | |
| | | AGTACCACTTCCACAGGAAACAAAA | |
| | | ATTTCCTTGCAGGACGCTTTAGTCAC | |
| | | TTG (SEQ ID NO: 16) | |
| | Galectin-9 | ATGGCATTTTCAGGATCACAAGCTC | MAFSGSQAPYLSPAVPFSGT |
| | | CATACTTGAGCCCAGCAGTGCCATT | IQGGLQDGLQITVNGTVLSS |
| | | TTCTGGCACGATTCAAGGCGGACTG | SGTRFAVNFQTGFSGNDIAF |
| | | CAAGACGGCTTGCAGATAACAGTCA | HFNPRFEDGGYVVCNTRQN |
| | | ACGGAACAGTACTGTCAAGTAGCGG | GSWGPEERKTHMPFQKGM |
| | | TACACGGTTCGCGGTGAACTTTCAG | PFDLCFLVQSSDFKVMVNG |
| | | ACTGGATTTTCTGGCAATGACATCG | ILFVQYFHRVPFHRVDTISV |
| | | CATTCCACTTCAATCCAAGGTTCGA | NGSVQLSYISFQNPRTVPVQ |
| | | AGATGGAGGTTATGTTGTTTGCAAT | PAFSTVPFSQPVCFPPRPRG |
| | | ACTAGGCAAAACGGCAGTTGGGGGC | RRQKPPGVWPANPAPITQT |
| | | CCGAGGAGCGGAAAACCCACATGCC | VIHTVQSAPGQMFSTPAIPP |
| | | ATTCCAGAAAGGGATGCCGTTCGAT | MMYPHPAYPMPFITTILGGL |
| | | CTCTGCTTTCTTGTTCAGAGTTCAGA | YPSKSILLSGTVLPSAQRFHI |
| | | TTTCAAAGTTATGGTCAATGGCATA | NLCSGNHIAFHLNPRFDEN |
| | | TTGTTCGTACAATATTTCCATCGAGT | AVVRNTQIDNSWGSEERSL |
| | | GCCCTTCCATAGGGTCGACACTATC | PRKMPFVRGQSFSVWILCE |
| | | AGTGTCAACGGTTCTGTCCAACTTTC | AHCLKVAVDGQHLFEYYH |
| | | CTATATATCCTTCCAGAATCCTCGAA | RLRNLPTINRLEVGGDIQLT |
| | | CTGTACCTGTGCAACCGGCGTTTTCA | HVQT (SEQ ID NO: 29) |
| | | ACCGTCCCGTTCAGCCAGCCCGTGT | |
| | | GCTTCCCCCCGAGACCAAGGGGTAG | |
| | | GCGACAGAAACCACCGGGTGTCTGG | |
| | | CCAGCAAACCCGGCCCCTATCACGC | |
| | | AAACAGTGATACATACTGTACAGAG | |
| | | CGCACCTGGACAAATGTTCAGCACA | |
| | | CCTGCCATACCTCCCATGATGTATCC | |
| | | CCACCCTGCGTACCCCATGCCATTC | |
| | | ATCACAACCATACTCGGCGGACTGT | |
| | | ACCCCTCTAAGTCCATCCTCCTTTCT | |
| | | GGTACCGTCCTCCCGAGCGCACAGC | |
| | | GATTCCACATCAATTTGTGCTCTGGT | |
| | | AACCATATCGCTTTCCATTTGAACCC | |
| | | ACGATTCGACGAAAACGCGGTAGTA | |
| | | AGGAATACACAAATTGACAACTCTT | |
| | | GGGGTAGTGAAGAACGCTCCTTGCC | |
| | | ACGGAAAATGCCGTTTGTCCGAGGC | |
| | | CAGAGCTTTAGTGTGTGGATTCTCTG | |
| | | TGAGGCACACTGTCTTAAGGTAGCC | |
| | | GTTGATGGGCAGCATCTCTTTGAAT | |
| | | ACTATCACAGGCTTCGGAACCTCCC | |
| | | GACAATCAATCGGCTGGAAGTTGGG | |
| | | GGGGATATACAGTTGACTCACGTGC | |
| | | AAACC (SEQ ID NO: 17) | |
| | LIF | ATGAAAGTTCTTGCCGCAGGGGTGG | MKVLAAGVVPLLLVLHWK |
| | | TTCCTCTGTTGCTCGTCTTGCACTGG | HGAGSPLPITPVNATCAIRH |
| | | AAACACGGGGCAGGGAGCCCGCTTC | PCHNNLMNQIRSQLAQLNG |
| | | CCATTACGCCTGTGAATGCAACGTG | SANALFILYYTAQGEPFPNN |
| | | CGCAATTAGGCATCCGTGCCATAAT | LDKLCGPNVTDFPPFHANG |
| | | AATCTGATGAACCAGATTAGGTCCC | TEKAKLVELYRIVVYLGTS |
| | | AACTCGCACAGCTCAATGGTTCAGC | LGNITRDQKILNPSALSLHS |
| | | GAACGCGCTTTTTATCTTGTATTATA | KLNATADILRGLLSNVLCR |
| | | CGGCACAGGGCGAACCGTTTCCAAA | LCSKYHVGHVDVTYGPDTS |
| | | TAACCTTGATAAACTGTGCGGGCCG | GKDVFQKKKLGCQLLGKY |
| | | AACGTCACCGACTTCCCGCCATTCC | KQIIAVLAQAF (SEQ ID NO: |
| | | ATGCGAACGGCACGGAGAAAGCAA | 30) |
| | | AACTCGTAGAGCTGTATCGGATTGT | |
| | | AGTATATCTGGGCACAAGCCTTGGC | |
| | | AACATAACACGGGACCAAAAAATTT | |
| | | TGAACCCCTCAGCTTTGAGTCTCCAC | |
| | | AGTAAACTCAATGCGACAGCAGATA | |
| | | TTCTGAGGGGGCTCCTGTCAAATGT | |
| | | GCTTTGCAGACTGTGCTCTAAGTAC | |
| | | CATGTTGGGCATGTTGACGTAACGT | |
| | | ACGGGCCTGACACTTCCGGGAAAGA | |
| | | CGTATTTCAGAAAAAGAAGCTCGGC | |
| | | TGCCAACTCCTGGGCAAATACAAGC | |
| | | AGATCATAGCCGTTCTTGCCCAGGC | |
| | | GTTC (SEQ ID NO: 18) | |
| | HLA-G5 | ATGGTGGTTATGGCACCAAGGACTC | MVVMAPRTLFLLLSGALTL |
| | | TCTTTTTGTTGCTCAGCGGGGCGTTG | TETWAGSHSMRYFSAAVSR |
| | | ACTCTCACAGAAACGTGGGCTGGTA | PGRGEPRFIAMGYVDDTQF |
| | | GCCATTCTATGCGATATTTCAGCGCC | VRFDSDSACPRMEPRAPWV |
| | | GCAGTGTCAAGACCGGGGCGGGGTG | EQEGPEYWEEETRNTKAHA |
| | | AACCGAGATTTATAGCTATGGGTTA | QTDRMNLQTLRGYYNQSE |
| | | CGTGGATGACACCCAGTTTGTGCGG | ASSHTLQWMIGCDLGSDGR |
| | | TTCGATAGTGATTCTGCGTGCCCAA | LLRGYEQYAYDGKDYLAL |
| | | GGATGGAACCCCGCGCACCGTGGGT | NEDLRSWTAADTAAQISKR |
| | | TGAACAAGAGGGTCCCGAATACTGG | KCEAANVAEQRRAYLEGT |
| | | GAAGAAGAGACTCGAAATACAAAA | CVEWLHRYLENGKEMLQR |
| | | GCACACGCCCAAACCGACAGAATGA | ADPPKTHVTHHPVFDYEAT |
| | | ACTTGCAGACTTTGCGAGGATACTA | LRCWALGFYPAEIILTWQR |
| | | TAATCAGAGCGAGGCAAGCAGTCAT | DGEDQTQDVELVETRPAGD |
| | | ACCCTTCAGTGGATGATTGGGTGCG | GTFQKWAAVVVPSGEEQR |
| | | ATCTTGGCTCAGACGGACGGCTCCT | YTCHVQHEGLPEPLMLRWS |
| | | GCGGGGGTATGAACAGTATGCTTAC | KEGDGGIMSVRESRSLSEDL |
| | | GATGGAAAAGACTACCTGGCTCTGA | (SEQ ID NO: 31) |
| | | ACGAGGATCTGAGGTCCTGGACAGC | |
| | | TGCCGATACCGCTGCACAGATATCT | |
| | | AAGAGAAAATGCGAGGCGGCCAAT | |
| | | GTCGCCGAACAGCGCAGAGCGTATT | |
| | | TGGAGGGAACGTGCGTAGAGTGGCT | |
| | | CCACAGATATCTGGAGAACGGAAAA | |
| | | GAAATGCTTCAACGCGCGGACCCTC | |
| | | CTAAAACCCACGTGACTCATCATCC | |
| | | TGTTTTCGATTATGAGGCCACGCTG | |
| | | AGATGTTGGGCTTTGGGATTTTATCC | |
| | | TGCGGAAATCATCCTTACCTGGCAG | |
| | | CGAGATGGTGAGGACCAGACCCAA | |
| | | GATGTCGAATTGGTGGAAACACGAC | |
| | | CAGCAGGTGACGGCACGTTTCAAAA | |
| | | ATGGGCGGCCGTAGTGGTACCTTCC | |
| | | GGAGAGGAGCAGCGATATACTTGTC | |
| | | ACGTTCAACATGAGGGACTCCCTGA | |
| | | GCCACTGATGCTGAGGTGGTCCAAG | |
| | | GAAGGAGACGGTGGCATAATGTCAG | |
| | | TCCGCGAGAGCCGATCTCTTTCCGA | |
| | | AGATCTG (SEQ ID NO: 19) | |
| | IL-1 RA | | MEICRGLRSHLITLLLFLFHS |
| | | | ETICRPSGRKSSKMQAFRIW |
| | | | DVNQKTFYLRNNQLVAGY |
| | | | LQGPNVNLEEKIDVVPIEPH |
| | | | ALFLGIHGGKMCLSCVKSG |
| | | | DETRLQLEAVNITDLSENRK |
| | | | QDKRFAFIRSDSGPTTSFES |
| | | | AACPGWFLCTAMEADQPV |
| | | | SLTNMPDEGVMVTKFYFQE |
| | | | DE (SEQ ID NO: 32) |
| | Emapalumab | | NFMLTQPHSVSESPGKTVTI |
| | light | | SCTRSSGSIASNYVQWYQQ |
| | chain | | RPGSSPTTVIYEDNQRPSGV |
| | | | PDRFSGSIDSSSNSASLTISG |
| | | | LKTEDEADYYCQSYDGSNR |
| | | | WMFGGGTKLTVLGQPKAA |
| | | | PSVTLFPPSSEELQANKATL |
| | | | VCLISDFYPGAVTVAWKAD |
| | | | SSPVKAGVETTTPSKQSNN |
| | | | KYAASSYLSLTPEQWKSHR |
| | | | SYSCQVTHEGSTVEKTVAP |
| | | | TECS (SEQ ID NO: 33) |
| | Emapalumab | | EVQLLESGGGLVQPGGSLR |
| | heavy | | LSCAASGFTFSSYAMSWVR |
| | chain | | QAPGKGLEWVSAISGSGGS |
| | | | TYYADSVKGRFTISRDNSK |
| | | | NTLYLQMNSLRAEDTAVY |
| | | | YCAKDGSSGWYVPHWFDP |
| | | | WGQGTLVTVSSASTKGPSV |
| | | | FPLAPSSKSTSGGTAALGCL |
| | | | VKDYFPEPVTVSWNSGALT |
| | | | SGVHTFPAVLQSSGLYSLSS |
| | | | VVTVPSSSLGTQTYICNVNH |
| | | | KPSNTKVDKRVEPKSCDKT |
| | | | HTCPPCPAPELLGGPSVFLF |
| | | | PPKPKDTLMISRTPEVTCVV |
| | | | VDVSHEDPEVKFNWYVDG |
| | | | VEVHNAKTKPREEQYNSTY |
| | | | RVVSVLTVLHQDWLNGKE |
| | | | YKCKVSNKALPAPIEKTISK |
| | | | AKGQPREPQVYTLPPSREE |
| | | | MTKNQVSLTCLVKGFYPSD |
| | | | IAVEWESNGQPENNYKTTP |
| | | | PVLDSDGSFFLYSKLTVDKS |
| | | | RWQQGNVFSCSVMHEALH |
| | | | NHYTQKSLSLSPGK (SEQ ID |
| | | | NO: 34) |
| | Ustekinumab | | DIQMTQSPSSLSASVGDRVT |
| | light | | ITCRASQGISSWLAWYQQK |
| | chain | | PEKAPKSLIYAASSLQSGVP |
| | | | SRFSGSGSGTDFTLTISSLQP |
| | | | EDFATYYCQQYNIYPYTFG |
| | | | QGTKLEIKRTVAAPSVFIFPP |
| | | | SDEQLKSGTASVVCLLNNF |
| | | | YPREAKVQWKVDNALQSG |
| | | | NSQESVTEQDSKDSTYSLSS |
| | | | TLTLSKADYEKHKVYACEV |
| | | | THQGLSSPVTKSFNRGEC |
| | | | (SEQ ID NO: 35) |
| | Ustekinumab | | EVQLVQSGAEVKKPGESLK |
| | heavy chain | | ISCKGSGYSFTTYWLGWVR |
| | | | QMPGKGLDWIGIMSPVDSD |
| | | | IRYSPSFQGQVTMSVDKSIT |
| | | | TAYLQWNSLKASDTAMYY |
| | | | CARRRPGQGYFDFWGQGT |
| | | | LVTVSSSSTKGPSVFPLAPSS |
| | | | KSTSGGTAALGCLVKDYFP |
| | | | EPVTVSWNSGALTSGVHTF |
| | | | PAVLQSSGLYSLSSVVTVPS |
| | | | SSLGTQTYICNVNHKPSNTK |
| | | | VDKRVEPKSCDKTH (SEQ |
| | | | ID NO: 36) |
| | Tumor necrosis | | MAPVAVWAALAVGLELW |
| | factor-binding | | AAAHALPAQVAFTPYAPEP |
| | protein 2 | | GSTCRLREYYDQTAQMCCS |
| | (targeting | | KCSPGQHAKVFCTKTSDTV |
| | domain | | CDSCEDSTYTQLWNWVPE |
| | of Embrel) | | CLSCGSRCSSDQVETQACT |
| | | | REQNRICTCRPGWYCALSK |
| | | | QEGCRLCAPLRKCRPGFGV |
| | | | ARPGTETSDVVCKPCAPGT |
| | | | FSNTTSSTDICRPHQICNVV |
| | | | AIPGNASMDAVCTSTSPTRS |
| | | | MAPGAVHLPQPVSTRSQHT |
| | | | QPTPEPSTAPSTSFLLPMGPS |
| | | | PPAEGSTGD (SEQ ID NO: |
| | | | 37) |
| | anti-TNFalpha | | QVQLQDSGGGLVQAGGSL |
| | Nanobody ® | | RLSCAASGGTFSSIIMAWFR |
| | (mouse | | QAPGKEREFVGAVSWSGGT |
| | specific) | | TVYADSVLGRFEISRDSARK |
| | | | SVYLQMNSLKPEDTAVYYC |
| | | | AARPYQKYNWASASYNVW |
| | | | GQGTQVTVS (SEQ ID NO: |
| | | | 30) |
| | Adalimumab | | DIQMTQSPSSLSASVGDRVT |
| | light chain | | ITCRASQGIRNYLAWYQQK |
| | | | PGKAPKLLIYAASTLQSGVP |
| | | | SRFSGSGSGTDFTLTISSLQP |
| | | | EDVATYYCQRYNRAPYTFG |
| | | | QGTKVEIKRTVAAPSVFIFP |
| | | | PSDEQLKSGTASVVCLLNN |
| | | | FYPREAKVQWKVDNALQS |
| | | | GNSQESVTEQDSKDSTYSLS |
| | | | STLTLSKADYEKHKVYACE |
| | | | VTHQGLSSPVTKSFNRGEC |
| | | | (SEQ ID NO: 39) |
| | Adalimumab | | EVQLVESGGGLVQPGRSLR |
| | heavy chain | | LSCAASGFTFDDYAMHWV |
| | | | RQAPGKGLEWVSAITWNSG |
| | | | HIDYADSVEGRFTISRDNAK |
| | | | NSLYLQMNSLRAEDTAVY |
| | | | YCAKVSYLSTASSLDYWGQ |
| | | | GTLVTVSSASTKGPSVFPLA |
| | | | PSSKSTSGGTAALGCLVKD |
| | | | YFPEPVTVSWNSGALTSGV |
| | | | HTFPAVLQSSGLYSLSSVVT |
| | | | VPSSSLGTQTYICNVNHKPS |
| | | | NTKVDKKVEPKSC (SEQ ID |
| | | | NO: 40) |
| | Brazikumab light | | QSVLTQPPSVSGAPGQRVTI |
| | chain | | SCTGSSSNTGAGYDVHWY |
| | | | QQVPGTAPKLLIYGSGNRPS |
| | | | GVPDRFSGSKSGTSASLAIT |
| | | | GLQAEDEADYYCQSYDSSL |
| | | | SGWVFGGGTRLTVLGQPK |
| | | | AAPSVTLFPPSSEELQANKA |
| | | | TLVCLISDFYPGAVTVAWK |
| | | | ADSSPVKAGVETTTPSKQS |
| | | | NNKYAASSYLSLTPEQWKS |
| | | | HRSYSCQVTHEGSTVEKTV |
| | | | APTECS (SEQ ID NO: 41) |
| | Brazikumab | | QVQLVESGGGVVQPGRSLR |
| | heavy chain | | LSCAASGFTFSSYGMHWVR |
| | | | QAPGKGLEWVAVIWYDGS |
| | | | NEYYADSVKGRFTISRDNS |
| | | | KNTLYLQMNSLRAEDTAV |
| | | | YYCARDRGYTSSWYPDAF |
| | | | DIWGQGTMVTVSSASTKGP |
| | | | SVFPLAPCSRSTSESTAALG |
| | | | CLVKDYFPEPVTVSWNSGA |
| | | | LTSGVHTFPAVLQSSGLYSL |
| | | | SSVVTVPSSNFGTQTYTCN |
| | | | VDHKPSNTKVDKTVERKCC |
| | | | VECPPCPAPPVAGPSVFLFP |
| | | | PKPKDTLMISRTPEVTCVVV |
| | | | DVSHEDPEVQFNWYVDGV |
| | | | EVHNAKTKPREEQFNSTFR |
| | | | VVSVLTVVHQDWLNGKEY |
| | | | KCKVSNKGLPAPIEKTISKT |
| | | | KGQPREPQVYTLPPSREEM |
| | | | TKNQVSLTCLVKGFYPSDIA |
| | | | VEWESNGQPENNYKTTPPM |
| | | | LDSDGSFFLYSKLTVDKSR |
| | | | WQQGNVFSCSVMHEALHN |
| | | | HYTQKSLSLSPGK (SEQ ID |
| | | | NO: 42) |
| | IDO | | MAHAMENSWTISKEYHIDE |
| | | | EVGFALPNPQENLPDFYND |
| | | | WMFIAKHLPDLIESGQLRER |
| | | | VEKLNMLSIDHLTDHKSQR |
| | | | LARLVLGCITMAYVWGKG |
| | | | HGDVRKVLPRNIAVPYCQL |
| | | | SKKLELPPILVYADCVLAN |
| | | | WKKKDPNKPLTYENMDVL |
| | | | FSFRDGDCSKGFFLVSLLVE |
| | | | IAAASAIKVIPTVFKAMQM |
| | | | QERDTLLKALLEIASCLEKA |
| | | | LQVFHQIHDHVNPKAFFSV |
| | | | LRIYLSGWKGNPQLSDGLV |
| | | | YEGFWEDPKEFAGGSAGQS |
| | | | SVFQCFDVLLGIQQTAGGG |
| | | | HAAQFLQDMRRYMPPAHR |
| | | | NFLCSLESNPSVREFVLSKG |
| | | | DAGLREAYDACVKALVSL |
| | | | RSYHLQIVTKYILIPASQQP |
| | | | KENKTSEDPSKLEAKGTGG |
| | | | TDLMNFLKTVRSTTEKSLL |
| | | | KEG (SEQ ID NO: 43) |
| | iNOS | | MACPWKFLFKTKFHQYAM |
| | | | NGEKDINNNVEKAPCATSS |
| | | | PVTQDDLQYHNLSKQQNES |
| | | | PQPLVETGKKSPESLVKLD |
| | | | ATPLSSPRHVRIKNWGSGM |
| | | | TFQDTLHHKAKGILTCRSKS |
| | | | CLGSIMTPKSLTRGPRDKPT |
| | | | PPDELLPQAIEFVNQYYGSF |
| | | | KEAKIEEHLARVEAVTKEIE |
| | | | TTGTYQLTGDELIFATKQA |
| | | | WRNAPRCIGRIQWSNLQVF |
| | | | DARSCSTAREMFEHICRHV |
| | | | RYSTNNGNIRSAITVFPQRS |
| | | | DGKHDFRVWNAQLIRYAG |
| | | | YQMPDGSIRGDPANVEFTQ |
| | | | LCIDLGWKPKYGRFDVVPL |
| | | | VLQANGRDPELFEIPPDLVL |
| | | | EVAMEHPKYEWFRELELK |
| | | | WYALPAVANMLLEVGGLE |
| | | | FPGCPFNGWYMGTEIGVRD |
| | | | FCDVQRYNILEEVGRRMGL |
| | | | ETHKLASLWKDQAVVEINI |
| | | | AVLHSFQKQNVTIMDHHSA |
| | | | AESFMKYMQNEYRSRGGC |
| | | | PADWIWLVPPMSGSITPVFH |
| | | | QEMLNYVLSPFYYYQVEA |
| | | | WKTHVWQDEKRRPKRREIP |
| | | | LKVLVKAVLFACMLMRKT |
| | | | MASRVRVTILFATETGKSE |
| | | | ALAWDLGALFSCAFNPKVV |
| | | | CMDKYRLSCLEEERLLLVV |
| | | | TSTFGNGDCPGNGEKLKKS |
| | | | LFMLKELNNKFRYAVFGLG |
| | | | SSMYPRFCAFAHDIDQKLS |
| | | | HLGASQLTPMGEGDELSGQ |
| | | | EDAFRSWAVQTFKAACETF |
| | | | DVRGKQHIQIPKLYTSNVT |
| | | | WDPHHYRLVQDSQPLDLSK |
| | | | ALSSMHAKNVFTMRLKSR |
| | | | QNLQSPTSSRATILVELSCE |
| | | | DGQGLNYLPGEHLGVCPGN |
| | | | QPALVQGILERVVDGPTPH |
| | | | QTVRLEALDESGSYWVSDK |
| | | | RLPPCSLSQALTYFLDITTPP |
| | | | TQLLLQKLAQVATEEPERQ |
| | | | RLEALCQPSEYSKWKFTNS |
| | | | PTFLEVLEEFPSLRVSAGFL |
| | | | LSQLPILKPRFYSISSSRDHT |
| | | | PTEIHLTVAVVTYHTRDGQ |
| | | | GPLHHGVCSTWLNSLKPQD |
| | | | PVPCFVRNASGFHLPEDPSH |
| | | | PCILIGPGTGIAPFRSFWQQR |
| | | | LHDSQHKGVRGGRMTLVF |
| | | | GCRRPDEDHIYQEEMLEMA |
| | | | QKGVLHAVHTAYSRLPGKP |
| | | | KVYVQDILRQQLASEVLRV |
| | | | LHKEPGHLYVCGDVRMAR |
| | | | DVAHTLKQLVAAKLKLNE |
| | | | EQVEDYFFQLKSQKRYHED |
| | | | IFGAVFPYEAKKDRVAVQP |
| | | | SSLEMSAL (SEQ ID NO: 44) |
| | COX2 | | MLARALLLCAVLALSHTAN |
| | | | PCCSHPCQNRGVCMSVGFD |
| | | | QYKCDCTRTGFYGENCSTP |
| | | | EFLTRIKLFLKPTPNTVHYIL |
| | | | THFKGFWNVVNNIPFLRNA |
| | | | IMSYVLTSRSHLIDSPPTYN |
| | | | ADYGYKSWEAFSNLSYYTR |
| | | | ALPPVPDDCPTPLGVKGKK |
| | | | QLPDSNEIVEKLLLRRKFIP |
| | | | DPQGSNMMFAFFAQHFTH |
| | | | QFFKTDHKRGPAFTNGLGH |
| | | | GVDLNHIYGETLARQRKLR |
| | | | LFKDGKMKYQIIDGEMYPP |
| | | | TVKDTQAEMIYPPQVPEHL |
| | | | RFAVGQEVFGLVPGLMMY |
| | | | ATIWLREHNRVCDVLKQEH |
| | | | PEWGDEQLFQTSRLILIGETI |
| | | | KIVIEDYVQHLSGYHFKLKF |
| | | | DPELLFNKQFQYQNRIAAEF |
| | | | NTLYHWHPLLPDTFQIHDQ |
| | | | KYNYQQFIYNNSILLEHGIT |
| | | | QFVESFTRQIAGRVAGGRN |
| | | | VPPAVQKVSQASIDQSRQM |
| | | | KYQSFNEYRKRFMLKPYES |
| | | | FEELTGEKEMSAELEALYG |
| | | | DIDAVELYPALLVEKPRPD |
| | | | AIFGETMVEVGAPFSLKGL |
| | | | MGNVICSPAYWKPSTFGGE |
| | | | VGFQIINTASIQSLICNNVKG |
| | | | CPFTSFSVPDPELIKTVTINA |
| | | | SSSRSGLDDINPTVLLKERS |
| | | | TEL (SEQ ID NO: 45) |
| | HO1 (heme | | MERPQPDSMPQDLSEALKE |
| | oxygenase-1) | | ATKEVHTQAENAEFMRNF |
| | | | QKGQVTRDGFKLVMASLY |
| | | | HIYVALEEEIERNKESPVFA |
| | | | PVYFPEELHRKAALEQDLA |
| | | | FWYGPRWQEVIPYTPAMQ |
| | | | RYVKRLHEVGRTEPELLVA |
| | | | HAYTRYLGDLSGGQVLKKI |
| | | | AQKALDLPSSGEGLAFFTFP |
| | | | NIASATKFKQLYRSRMNSL |
| | | | EMTPAVRQRVIEEAKTAFL |
| | | | LNIQLFEELQELLTHDTKDQ |
| | | | SPSRAPGLRQRASNKVQDS |
| | | | APVETPRGKPPLNTRSQAPL |
| | | | LRWVLTLSFLVATVAVGLY |
| | | | AM (SEQ ID NO: 46) |
| | HIF-2-alpha | | MTADKEKKRSSSERRKEKS |
| | | | RDAARCRRSKETEVFYELA |
| | | | HELPLPHSVSSHLDKASIMR |
| | | | LAISFLRTHKLLSSVCSENES |
| | | | EAEADQQMDNLYLKALEG |
| | | | FIAVVTQDGDMIFLSENISK |
| | | | FMGLTQVELTGHSIFDFTHP |
| | | | CDHEEIRENLSLKNGSGFGK |
| | | | KSKDMSTERDFFMRMKCT |
| | | | VTNRGRTVNLKSATWKVL |
| | | | HCTGQVKVYNNCPPHNSLC |
| | | | GYKEPLLSCLIIMCEPIQHPS |
| | | | HMDIPLDSKTFLSRHSMDM |
| | | | KFTYCDDRITELIGYHPEEL |
| | | | LGRSAYEFYHALDSENMTK |
| | | | SHQNLCTKGQVVSGQYRM |
| | | | LAKHGGYVWLETQGTVIY |
| | | | NPRNLQPQCIMCVNYVLSEI |
| | | | EKNDVVFSMDQTESLFKPH |
| | | | LMAMNSIFDSSGKGAVSEK |
| | | | SNFLFTKLKEEPEELAQLAP |
| | | | TPGDAIISLDFGNQNFEESS |
| | | | AYGKAILPPSQPWATELRS |
| | | | HSTQSEAGSLPAFTVPQAA |
| | | | APGSTTPSATSSSSSCSTPNS |
| | | | PEDYYTSLDNDLKIEVIEKL |
| | | | FAMDTEAKDQCSTQTDFNE |
| | | | LDLETLAPYIPMDGEDFQLS |
| | | | PICPEERLLAENPQSTPQHC |
| | | | FSAMTNIFQPLAPVAPHSPF |
| | | | LLDKFQQQLESKKTEPEHR |
| | | | PMSSIFFDAGSKASLPPCCG |
| | | | QASTPLSSMGGRSNTQWPP |
| | | | DPPLHFGPTKWAVGDQRTE |
| | | | FLGAAPLGPPVSPPHVSTFK |
| | | | TRSAKGFGARGPDVLSPAM |
| | | | VALSNKLKLKRQLEYEEQA |
| | | | FQDLSGGDPPGGSTSHLMW |
| | | | KRMKNLRGGSCPLMPDKPL |
| | | | SANVPNDKFTQNPMRGLG |
| | | | HPLRHLPLPQPPSAISPGENS |
| | | | KSRFPPQCYATQYQDYSLS |
| | | | SAHKVSGMASLLGPSFESY |
| | | | LLPELTRYDCEVNVPVLGSS |
| | | | TLLQGGDLLRALDQAT |
| | | | (SEQ ID NO: 47) |
| Ex- | C-terminal myc | GGGTCTTCGGGAAGTGAGCAGAAGC | GSSGSEQKLISEEDL (SEQ |
| pression | tag (removable) | TGATCAGCGAGGAGGACCTGTAAGA | ID NO: 48) |
| Tag | | AGACTG (SEQ ID NO: 20) | |

Expression Vector: pL+MCS
ACGCGTGTAGTCTTATGCAATACTCTTGTAGTCTTGCAACATGGTAACGATGAGTTAGCAACATGC
CTTACAAGGAGAGAAAAAGCACCGTGCATGCCGATTGGTGGAAGTAAGGTGGTACGATCGTGCCT
TATTAGGAAGGCAACAGACGGGTCTGACATGGATTGGACGAACCACTGAATTGCCGCATTGCAGA
GATATTGTATTTAAGTGCCTAGCTCGATACAATAAACGGGTCTCTCTGGTTAGACCAGATCTGAGC
CTGGGAGCTCTCTGGCTAACTAGGGAACCCACTGCTTAAGCCTCAATAAAGCTTGCCTTGAGTGCT
TCAAGTAGTGTGTGCCCGTCTGTTGTGTGACTCTGGTAACTAGAGATCCCTCAGACCCTTTTAGTCA
GTGTGGAAAATCTCTAGCAGTGGCGCCCGAACAGGGACCTGAAAGCGAAAGGGAAACCAGAGCT
CTCTCGACGCAGGACTCGGCTTGCTGAAGCGCGCACGGCAAGAGGCGAGGGGCGGCGACTGGTGA
GTACGCCAAAAATTTTGACTAGCGGAGGCTAGAAGGAGAGAGATGGGTGCGAGAGCGTCAGTATT
AAGCGGGGGAGAATTAGATCGCGATGGGAAAAAATTCGGTTAAGGCCAGGGGGAAAGAAAAAAT
ATAAATTAAAACATATAGTATGGGCAAGCAGGGAGCTAGAACGATTCGCAGTTAATCCTGGCCTG
TTAGAAACATCAGAAGGCTGTAGACAAATACTGGGACAGCTACAACCATCCCTTCAGACAGGATC
AGAAGAACTTAGATCATTATATAATACAGTAGCAACCCTCTATTGTGTGCATCAAAGGATAGAGAT
AAAAGACACCAAGGAAGCTTTAGACAAGATAGAGGAAGAGCAAAACAAAAGTAAGACCACCGCA
CAGCAAGCGGCCACTGATCTTCAGACCTGGAGGAGGAGATATGAGGGACAATTGGAGAAGTGAAT
TATATAAATATAAAGTAGTAAAAATTGAACCATTAGGAGTAGCACCCACCAAGGCAAAGAGAAGA
GTGGTGCAGAGAGAAAAAAGAGCAGTGGGAATAGGAGCTTTGTTCCTTGGGTTCTTGGGAGCAGC
AGGAAGCACTATGGGCGCAGCCTCAATGACGCTGACGGTACAGGCCAGACAATTATTGTCTGGTA
TAGTGCAGCAGCAGAACAATTTGCTGAGGGCTATTGAGGCGCAACAGCATCTGTTGCAACTCACA
GTCTGGGGCATCAAGCAGCTCCAGGCAAGAATCCTGGCTGTGGAAAGATACCTAAAGGATCAACA
GCTCCTGGGGATTTGGGGTTGCTCTGGAAAACTCATTTGCACCACTGCTGTGCCTTGGAATGCTAG
TTGGAGTAATAAATCTCTGGAACAGATTGGAATCACACGACCTGGATGGAGTGGGACAGAGAAAT
TAACAATTACACAAGCTTAATACACTCCTTAATTGAAGAATCGCAAAACCAGCAAGAAAAGAATG
AACAAGAATTATTGGAATTAGATAAATGGGCAAGTTTGTGGAATTGGTTTAACATAACAAATTGG
CTGTGGTATATAAAATTATTCATAATGATAGTAGGAGGCTTGGTAGGTTTAAGAATAGTTTTTGCT
GTACTTTCTATAGTGAATAGAGTTAGGCAGGGATATTCACCATTATCGTTTCAGACCCACCTCCCA
ACCCCGAGGGGACCCGACAGGCCCGAAGGAATAGAAGAAGAAGGTGGAGAGAGAGACAGAGAC
AGATCCATTCGATTAGTGAACGGATCTCGACGGTATCGGTTAACTTTTAAAAGAAAAGGGGGGAT
TGGGGGGTACAGTGCAGGGGAAAGAATAGTAGACATAATAGCAACAGACATACAAACTAAAGAA
TTACAAAAACAAATTACAAAAATCAAAATTTTATCTCGACATGGTGGCGACCGGTAGCGCTAGCG
GATCGATAAGCTTGATATCGCCTGCAGCCGAATTCCTTGACTTGGGATCCGCGTCAAGTGGAGCAA
GGCAGGTGGACAGTCCTGCAGGCATGCGTGACTGACTGAGGCCGCGACTCTAGTTTAAACTGCGT
GACTGACTCTAGAAGATCCGGCAGTGCGGCCGCGTCGACAATCAACCTCTGGATTACAAAATTTGT
GAAAGATTGACTGGTATTCTTAACTATGTTGCTCCTTTTACGCTATGTGGATACGCTGCTTTAATGC
CTTTGTATCATGCTATTGCTTCCCGTATGGCTTTCATTTTCTCCTCCTTGTATAAATCCTGGTTGCTG
TCTCTTTATGAGGAGTTGTGGCCCGTTGTCAGGCAACGTGGCGTGGTGTGCACTGTGTTTGCTGAC
GCAACCCCCACTGGTTGGGGCATTGCCACCACCTGTCAGCTCCTTTCCGGGACTTTCGCTTTCCCCC
TCCCTATTGCCACGGCGGAACTCATCGCCGCCTGCCTTGCCCGCTGCTGGACAGGGGCTCGGCTGT
TGGGCACTGACAATTCCGTGGTGTTGTCGGGGAAATCATCGTCCTTTCCTTGGCTGCTCGCCTGTGT
TGCCACCTGGATTCTGCGCGGGACGTCCTTCTGCTACGTCCCTTCGGCCCTCAATCCAGCGGACCTT
CCTTCCCGCGGCCTGCTGCCGGCTCTGCGGCCTCTTCCGCGTCTTCGCCTTCGCCCTCAGACGAGTC
GGATCTCCCTTTGGGCCGCCTCCCCGCCTGGTACCTTTAAGACCAATGACTTACAAGGCAGCTGTA
GATCTTAGCCACTTTTTAAAAGAAAAGGGGGGACTGGAAGGGCTAATTCACTCCCAACGAAAATA
AGATCTGCTTTTTGCTTGTACTGGGTCTCTCTGGTTAGACCAGATCTGAGCCTGGGAGCTCTCTGGC
TAACTAGGGAACCCACTGCTTAAGCCTCAATAAAGCTTGCCTTGAGTGCTTCAAGTAGTGTGTGCC
CGTCTGTTGTGTGACTCTGGTAACTAGAGATCCCTCAGACCCTTTTAGTCAGTGTGGAAAATCTCTA
GCAGTAGTAGTTCATGTCATCTTATTATTCAGTATTTATAACTTGCAAAGAAATGAATATCAGAGA
GTGAGAGGAACTTGTTTATTGCAGCTTATAATGGTTACAAATAAAGCAATAGCATCACAAATTTCA
CAAATAAAGCATTTTTTTCACTGCATTCTAGTTGTGGTTTGTCCAAACTCATCAATGTATCTTATCA
TGTCTGGCTCTAGCTATCCCGCCCCTAACTCCGCCCAGTTCCGCCCATTCTCCGCCCCATGGCTGAC
TAATTTTTTTTATTTATGCAGAGGCCGAGGCCGCCTCGGCCTCTGAGCTATTCCAGAAGTAGTGAG
GAGGCTTTTTTGGAGGCCTAGACTTTTGCAGAGACGGCCCAAATTCGTAATCATGGTCATAGCTGT
TTCCTGTGTGAAATTGTTATCCGCTCACAATTCCACACAACATACGAGCCGGAAGCATAAAGTGTA
AAGCCTGGGGTGCCTAATGAGTGAGCTAACTCACATTAATTGCGTTGCGCTCACTGCCCGCTTTCC
AGTCGGGAAACCTGTCGTGCCAGCTGCATTAATGAATCGGCCAACGCGCGGGGAGAGGCGGTTTG
CGTATTGGGCGCTCTTCCGCTTCCTCGCTCACTGACTCGCTGCGCTCGGTCGTTCGGCTGCGGCGAG
CGGTATCAGCTCACTCAAAGGCGGTAATACGGTTATCCACAGAATCAGGGGATAACGCAGGAAAG
AACATGTGAGCAAAAGGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTCAGAGGTGGCGAAACC
CGACAGGACTATAAAGATACCAGGCGTTTCCCCCTGGAAGCTCCCTCGTGCGCTCTCCTGTTCCGA
CCCTGCCGCTTACCGGATACCTGTCCGCCTTTCTCCCTTCGGGAAGCGTGGCGCTTTCTCATAGCTC
ACGCTGTAGGTATCTCAGTTCGGTGTAGGTCGTTCGCTCCAAGCTGGGCTGTGTGCACGAACCCCC
CGTTCAGCCCGACCGCTGCGCCTTATCCGGTAACTATCGTCTTGAGTCCAACCCGGTAAGACACGA
CTTATCGCCACTGGCAGCAGCCACTGGTAACAGGATTAGCAGAGCGAGGTATGTAGGCGGTGCTA
CAGAGTTCTTGAAGTGGTGGCCTAACTACGGCTACACTAGAAGGACAGTATTTGGTATCTGCGCTC
TGCTGAAGCCAGTTACCTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAAAAGGATCTCAAGAAGA
TCCTTTGATCTTTTCTACGGGGTCTGACGCTCAGTGGAACGAAAACTCACGTTAAGGGATTTTGGT
CATGAGATTATCAAAAAGGATCTTCACCTAGATCCTTTTAAATTAAAAATGAAGTTTTAAATCAAT
CTAAAGTATATATGAGTAAACTTGGTCTGACAGTTACCAATGCTTAATCAGTGAGGCACCTATCTC
AGCGATCTGTCTATTTCGTTCATCCATAGTTGCCTGACTCCCCGTCGTGTAGATAACTACGATACGG
GAGGGCTTACCATCTGGCCCCAGTGCTGCAATGATACCGCGAGACCCACGCTCACCGGCTCCAGAT
TTATCAGCAATAAACCAGCCAGCCGGAAGGGCCGAGCGCAGAAGTGGTCCTGCAACTTTATCCGC
CTCCATCCAGTCTATTAATTGTTGCCGGGAAGCTAGAGTAAGTAGTTCGCCAGTTAATAGTTTGCG
CAACGTTGTTGCCATTGCTACAGGCATCGTGGTGTCACGCTCGTCGTTTGGTATGGCTTCATTCAGC
TCCGGTTCCCAACGATCAAGGCGAGTTACATGATCCCCCATGTTGTGCAAAAAAGCGGTTAGCTCC
TTCGGTCCTCCGATCGTTGTCAGAAGTAAGTTGGCCGCAGTGTTATCACTCATGGTTATGGCAGCA
CTGCATAATTCTCTTACTGTCATGCCATCCGTAAGATGCTTTTCTGTGACTGGTGAGTACTCAACCA
AGTCATTCTGAGAATAGTGTATGCGGCGACCGAGTTGCTCTTGCCCGGCGTCAATACGGGATAATA
CCGCGCCACATAGCAGAACTTTAAAAGTGCTCATCATTGGAAAACGTTCTTCGGGGCGAAAACTCT
CAAGGATCTTACCGCTGTTGAGATCCAGTTCGATGTAACCCACTCGTGCACCCAACTGATCTTCAG
CATCTTTTACTTTCACCAGCGTTTCTGGGTGAGCAAAAACAGGAAGGCAAAATGCCGCAAAAAAG
GGAATAAGGGCGACACGGAAATGTTGAATACTCATACTCTTCCTTTTTCAATATTATTGAAGCATT
TATCAGGGTTATTGTCTCATGAGCGGATACATATTTGAATGTATTTAGAAAAATAAACAAATAGGG
GTTCCGCGCACATTTCCCCGAAAAGTGCCACCTGACGTCTAAGAAACCATTATTATCATGACATTA
ACCTATAAAAATAGGCGTATCACGAGGCCCTTTCGTCTCGCGCGTTTCGGTGATGACGGTGAAAAC
CTCTGACACATGCAGCTCCCGGAGACGGTCACAGCTTGTCTGTAAGCGGATGCCGGGAGCAGACA
AGCCCGTCAGGGCGCGTCAGCGGGTGTTGGCGGGTGTCGGGGCTGGCTTAACTATGCGGCATCAG
AGCAGATTGTACTGAGAGTGCACCATATGCGGTGTGAAATACCGCACAGATGCGTAAGGAGAAAA
TACCGCATCAGGCGCCATTCGCCATTCAGGCTGCGCAACTGTTGGGAAGGGCGATCGGTGCGGGC
CTCTTCGCTATTACGCCAGCTGGCGAAAGGGGGATGTGCTGCAAGGCGATTAAGTTGGGTAACGC
CAGGGTTTTCCCAGTCACGACGTTGTAAAACGACGGCCAGTGCCAAGCTG (SEQ ID NO: 49)

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A mesenchymal stem cell engineered to produce two anti-inflammatory cytokines at levels sufficient to inhibit an inflammatory response.

2. A mesenchymal stem cell of claim 1, wherein the inflammatory response is inhibited by at least 20% relative to a control, optionally wherein the control is an unmodified mesenchymal stem cell.

3. The mesenchymal stem cell of claim 1 or 2, wherein the anti-inflammatory cytokines are selected from IL-4, IL-10, and IL-22.

4. The mesenchymal stem cell of claim 3, wherein the anti-inflammatory cytokines are IL-4 and IL-10.

5. The mesenchymal stem cell of claim 3, wherein the anti-inflammatory cytokines are IL-4 and IL-22.

6. The mesenchymal stem cell of claim 3, wherein the anti-inflammatory cytokines are IL-10 and IL-22.

7. The mesenchymal stem cell of any one of claims 1-6, wherein the mesenchymal stem cell is derived from bone marrow, adipose tissue, or umbilical cord tissue.

8. The mesenchymal stem cell of any one of claims 1-7, wherein the anti-inflammatory cytokine levels are sufficient to induce a regulatory T cell immunophenotype.

9. The mesenchymal stem cell of any one of claims 1-8, wherein the anti-inflammatory cytokine levels are sufficient to inhibit production of inflammatory cytokine by stimulated T cells by at least 20% relative to a control, optionally wherein the control is an unmodified mesenchymal stem cell.

10. The mesenchymal stem cell of claim 9, wherein the inflammatory cytokines are selected from IFN-gamma, IL-17A, IL-1-beta, IL-6, and TNF-alpha.

11. The mesenchymal stem cell of claim 9 or 10, wherein the T cells are selected from CD8⁺ T cells, CD4⁺ T cells, gamma-delta T cells, and T regulatory cells.

12. The mesenchymal stem cell of any one of claims 1-11, wherein the mesenchymal stem cell is engineered to produce at least three anti-inflammatory cytokines at levels sufficient to inhibit an inflammatory response by at least 20% relative to a control, optionally wherein the control is an unmodified mesenchymal stem cell.

13. The mesenchymal stem cell of any one of claims 1-12, wherein the mesenchymal stem cell is engineered to express a homing molecule.

14. The mesenchymal stem cell of claim 13, wherein the homing molecule is selected from: anti-integrin alpha4,beta7; anti-MAdCAM; CCR9; CXCR4; SDF1; MMP-2; CXCR1; CXCR7; CCR2; and GPR15.

15. The mesenchymal stem cell of claim 14, wherein the homing molecule is selected from: CXCR4, CCR2, CCR9, and GPR15.

16. The mesenchymal stem cell of any one of claims 1-15, wherein the mesenchymal stem cell comprises:
(a) a nucleic acid comprising a promoter operably linked to a first nucleotide sequence encoding one of the two cytokines and a second nucleotide sequence encoding the other of the two cytokines, optionally wherein the first and second nucleotide sequence are separated by an intervening nucleotide sequence, optionally wherein the intervening sequence is an IRES sequence or encodes a 2A peptide;
(b) a nucleic acid comprising (i) a first promoter operably linked to a nucleotide sequence encoding one of the two cytokines and (ii) a second promoter operably linked to a nucleotide sequence encoding the other of the two cytokines; or
(c) a first nucleic acid comprising a first promoter operably linked to a nucleotide sequence encoding one of the two cytokines, and a second nucleic acid comprising a second promoter operably linked to a nucleotide sequence encoding the other of the two cytokines.

17. The mesenchymal stem cell of claim 16, wherein the promoter of (a), the first and/or second promoter of (b), and/or the first and/or second promoter of (c) is an inducible promoter.

18. The mesenchymal stem cell of claim 17, wherein the inducible promoter is a nuclear factor kappa-B (NF-κB)-responsive promoter.

19. The mesenchymal stem cell of any one of claims 16-18, wherein the nucleic acid of (a), the nucleic acid of (b), and/or the first and/or second nucleic acid of (c) further comprises a promoter operably linked to a nucleotide sequence encoding a reporter molecule.

20. A method comprising delivering to a subject a therapeutically effective amount of a preparation of mesenchymal stem cells engineered to produce two anti-inflammatory cytokines, wherein the therapeutically effective amount is sufficient to inhibit an inflammatory response in the subject.

21. The method of claim 20, wherein the inflammatory response is inhibited by at least 20% relative to a control, optionally wherein the control is a preparation of unmodified mesenchymal stem cells.

22. The method of claim 20 or 21, wherein the anti-inflammatory cytokines are selected from IL-4, IL-10, and IL-22.

23. The method of claim 22, wherein the anti-inflammatory cytokines are IL-4 and IL-10.

24. The method of claim 22, wherein the anti-inflammatory cytokines are IL-4 and IL-22.

25. The method of claim 22, wherein the anti-inflammatory cytokines are IL-10 and IL-22.

26. The method of any one of claims 20-25, wherein the mesenchymal stem cells are derived from bone marrow, adipose tissue, or umbilical cord tissue.

27. The method of any one of claims 20-26, wherein the therapeutically effective amount is sufficient to induce a regulatory T cell immunophenotype.

28. The method of any one of claims 20-27, wherein the therapeutically effective amount is sufficient to inhibit production of inflammatory cytokines by stimulated T cells by at least 20% relative to a control, optionally wherein the control is a preparation of unmodified mesenchymal stem cells.

29. The method of claim 28, wherein the inflammatory cytokines are selected from IFN-gamma, IL-17A, IL-1-beta, IL-6, and TNF-alpha.

30. The method of claim 28 or 29, wherein the T cells are selected from CD8⁺ T cells, CD4⁺ T cells, gamma-delta T cells, and T regulatory cells.

31. The method of any one of claims 20-30, wherein the mesenchymal stem cells are engineered to produce at least three anti-inflammatory cytokines.

32. The method of any one of claims 20-31, wherein the subject is symptomatic of having an inflammatory bowel disease.

33. The method of claim 32, wherein the subject has been diagnosed with having an inflammatory bowel disease.

34. The method of claim 32 or 33, wherein the inflammatory bowel disease is ulcerative colitis or Crohn's disease.

35. The method of any one of claims 20-34, wherein the therapeutically effective amount reduces weight loss in the subject by at least 20% relative to a control, optionally wherein the control is a preparation of unmodified mesenchymal stem cells.

36. The method of any one of claims 20-35, wherein the therapeutically effective amount reduces levels of lipocalin-2 in the subject by at least 20% relative to a control, optionally wherein the control is a preparation of unmodified mesenchymal stem cells.

37. The method of any one of claims 20-36, wherein the mesenchymal stem cells are engineered to express a homing molecule.

38. The method of claim 37, wherein the homing molecule is selected from: anti-integrin alpha4,beta7; anti-MAdCAM; CCR9; CXCR4; SDF1; MMP-2; CXCR1; CXCR7; CCR2; and GPR15.

39. The method of claim 38, wherein the homing molecule is selected from: CXCR4, CCR2, CCR9, and GPR15.

40. The method of any one of claims 20-39, wherein the mesenchymal stem cells comprise
(a) a nucleic acid comprising a promoter operably linked to a first nucleotide sequence encoding one of the two cytokines and a second nucleotide sequence encoding the other of the two cytokines, optionally wherein the first and second nucleotide sequence are separated by an intervening nucleotide sequence, optionally wherein the intervening sequence is an IRES sequence or encodes a 2A peptide;
(b) a nucleic acid comprising (i) a first promoter operably linked to a nucleotide sequence encoding one of the two cytokines and (ii) a second promoter operably linked to a nucleotide sequence encoding the other of the two cytokines; or
(c) a first nucleic acid comprising a first promoter operably linked to a nucleotide sequence encoding one of the two cytokines, and a second nucleic acid comprising a second promoter operably linked to a nucleotide sequence encoding the other of the two cytokines.

41. The method of claim 40, wherein the promoter of (a), the first and/or second promoter of (b), and/or the first and/or second promoter of (c) is an inducible promoter.

42. The method of claim 41, wherein the inducible promoter is a nuclear factor kappa-B (NF-κB)-responsive promoter.

43. An engineered nucleic acid comprising a nuclear factor kappa-B (NF-κB)-responsive promoter operably linked to a nucleotide sequence encoding an effector molecule.

44. The engineered nucleic acid of claim 43, wherein the effector molecule is an anti-inflammatory cytokine.

45. The engineered nucleic acid of claim 44, wherein the anti-inflammatory cytokine is selected from IL-4, IL-10, and IL-22.

46. A mesenchymal stem cell engineered to produce multiple effector molecules, at least two of which modulate different cell types of the immune system.
in vivo

47. A method of producing a multifunctional immunomodulatory cell, comprising
(a) delivering to a mesenchymal stem cell at least one engineered nucleic acid encoding at least two effector molecules, or
(b) delivering to a mesenchymal stem cell at least two engineered nucleic acids, each encoding at least one effector molecule,
wherein each effector molecule modulates a different cell type of the immune system or modulates different functions of a cell.

48. A method of modulating multiple cell types of the immune system of a subject, comprising delivering to the subject at least two mesenchymal stem cells, each engineered to produce an effector molecule, wherein at least two of the effector molecules modulate different cell types of the immune system.

49. A mesenchymal stem cell engineered to produce an effector molecule and a homing molecule at levels sufficient to inhibit an inflammatory response.

50. The mesenchymal stem cell of claim 49, wherein the effector molecule is selected from IL-4, IL-10, IL-35, PD-L1-Ig, anti-TNF-alpha, indoleamine 2,3-dioxygenase (IDO), alpha-1 antitrypsin, IL-22, IL-19, and IL-20.

51. The mesenchymal stem cell of claim 50, wherein the homing molecule is selected from anti-integrin alpha4,beta7; anti-MAdCAM; CCR9; CXCR4; SDF1; MMP-2; CXCR1; CXCR7; CCR2; and GPR15.


SEQUENCE LISTING (49 sequences)

SEQ ID NO: 1 - DNA, 588 bp, Artificial Sequence
  Synthetic polynucleotide
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 2 - DNA, 1179 bp, Artificial Sequence
  Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgccgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcgaccaccgagaatcggacgggggtagtctcaagctggccggcctgctctggtgcctgtcctcgcgccgccgtgtatcgccccgccccgggcggcaaggctggcccggtcggcaccagttgcgtgagcggaaagatggccgcttcccggtcctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 3 - DNA, 544 bp, Artificial Sequence
  Synthetic polynucleotide
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 4 - DNA, 399 bp, Artificial Sequence
  Synthetic polynucleotide
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 5 - DNA, 511 bp, Artificial Sequence
  Synthetic polynucleotide
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 6 - DNA, 408 bp, Artificial Sequence
  Synthetic polynucleotide
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 7 - DNA, 344 bp, Artificial Sequence
  Synthetic polynucleotide
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 8 - DNA, 1229 bp, Artificial Sequence
  Synthetic polynucleotide
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagac

SEQ ID NO: 9 - DNA, 870 bp, Artificial Sequence
  Synthetic polynucleotide
atgagaatttttgccgtgtttatttttatgacttactggcaccttcttaacgctttcacggttactgttccgaaggatctgtacgttgtagaatacggtagcaacatgactatagagtgcaaatttcccgttgagaaacaacttgatcttgccgccttgatcgtctactgggaaatggaggacaaaaatataatacagttcgttcatggagaggaggaccttaaagtacagcactcttcatacagacagcgcgcgcggcttttgaaagatcagctttctctgggcaacgcggctcttcaaattacagatgtcaaattgcaagatgctggagtatacagatgtatgatctcttacggtggcgcagattataagaggattacggtaaaagtaaacgccccctataacaaaatcaatcagaggattctggtcgtcgacccggtaacgagtgagcacgaattgacttgccaagctgaaggctacccgaaggcggaggtcatatggacttcctctgatcatcaagttttgtctggcaaaacgacaactaccaacagtaagagagaggaaaagttgttcaacgttacgtccacactcagaataaacacgaccactaacgagattttttactgcacgtttcgacgacttgacccggaagaaaatcacacagcagagcttgtgatccctgaactgcccctggcccatccaccaaatgaacgaactcatctggtcattctcggtgctattttgttgtgtctcggagtggcacttacctttatatttagactccgaaaaggtcgcatgatggacgtcaaaaagtgcggaatccaagacaccaacagtaaaaaacaatccgatactcatcttgaagaaaca

SEQ ID NO: 10 - DNA, 534 bp, Artificial Sequence
  Synthetic polynucleotide
atgcattctagcgcgttgctgtgttgcctcgtgctgctcactggggttcgggcctcccctggtcaaggaacccaatcagagaactcatgcacgcattttccggggaacttgccgaatatgttgcgagacctgcgcgatgcattttccagagtaaagacctttttccaaatgaaggaccagctcgataatttgttgctcaaagagagtttgctggaggactttaaaggttacctcggatgccaggctctgtctgagatgattcaattttatttggaggaagtaatgccgcaggcggaaaaccaggaccccgatataaaggctcatgtaaactctctgggtgaaaaccttaaaacactgagattgcgcctccgaagatgtcataggttccttccgtgcgaaaataagagcaaggctgttgaacaggtgaaaaatgcttttaacaaacttcaagagaaagggatttataaggcaatgtcagagtttgacattttcatcaactacatagaagcgtacatgacgatgaaaattcgcaat

SEQ ID NO: 11 - DNA, 597 bp, Artificial Sequence
  Synthetic polynucleotide
atgaactgcgtctgccggttggtattggtagtcttgtctttgtggccggatactgccgtcgctcccggccccccgcctggtcctccccgagtctcaccagacccgagagcagaactcgattctacggttctgttgacccggtcactgctggcggacacccggcaactcgccgcccaactgcgggacaaatttcctgcagacggcgatcacaacttggactctcttccaacacttgcaatgtcagctggcgcccttggtgctctgcaattgccgggggtcctgacgagactgcgagcggatctgctcagctacctgaggcacgttcaatggctgagacgggcgggaggaagctcacttaagacactggagcccgagctcggcaccttgcaagctcggctggatcggctcctgagacgattgcaacttctcatgtctcgactggcacttccacaaccaccccctgatccccccgcgcccccactggcccccccgtcatctgcgtggggaggcatcagagcagcccatgctattttggggggactccatctcacccttgattgggcggtgcggggcctccttctcttgaaaacgcggctt

SEQ ID NO: 12 - DNA, 438 bp, Artificial Sequence
  Synthetic polynucleotide
atgcatcccctgctcaatcccctcttgctggcgcttggcctcatggctctgctcctgacgactgtcatagctcttacatgcctgggtggtttcgcaagccctgggccagtcccgccgtcaacagcacttagagagctcatagaagaactcgtcaacatcacgcagaaccaaaaagccccgttgtgcaacggtagcatggtatggtcaatcaacctgacagcagggatgtattgtgccgctttggagtccttgattaatgtttccggttgcagtgcaattgagaaaacacagcgaatgctgtctggcttctgtcctcacaaagttagcgcagggcaatttagttccctccatgtaagggacactaaaatagaggtcgctcaattcgttaaggatttgcttcttcatttgaagaagctgttcagggagggcaggtttaat

SEQ ID NO: 13 - DNA, 459 bp, Artificial Sequence
  Synthetic polynucleotide
atggggctcacctcacagctcctgccgccgctctttttccttctcgcctgcgcgggtaattttgttcacggtcataagtgtgatataactctgcaggagataatcaagactcttaattctctcacagagcagaaaacactttgcactgagctgacggtcaccgacatcttcgctgcatctaagaataccaccgaaaaggaaacattttgccgggctgcgacagttttgcggcagttttactcccaccatgagaaagacacgcgatgccttggtgccacagctcagcaattccataggcataaacaattgattcgatttcttaagcggcttgatcgaaacctgtgggggcttgcggggttgaactcatgcccggttaaagaagcaaatcagtctactctggagaattttttggaacggcttaagacgattatgagagaaaaatactccaaatgttcctcc

SEQ ID NO: 14 - DNA, 1326 bp, Artificial Sequence
  Synthetic polynucleotide
atgacgccgcaacttttgctggcacttgtgttgtgggcttcttgtccaccttgctcagggcgcaaagggcctccggctgctttgacgttgccaagagtgcagtgccgggcctcccgataccctatagctgtggactgttcttggacattgccgccggccccgaactccacctcacccgtctccttcattgccacttaccgactgggaatggcagccaggggacacagttggccatgcctgcaacagacacctacttcaaccagctgtacgatcacagacgtccaacttttcagcatggcaccatacgttcttaacgtaactgcagtacatccgtgggggagttctagtagcttcgttccgttcataactgagcacatcataaaaccagacccacctgagggagtccgcttgtctcctcttgccgagaggcaacttcaagttcagtgggaaccgccggggtcttggccgtttcccgaaatattttcacttaaatactggattagatataaaaggcaaggtgcggcgagattccatcgggtcgggccaatagaagctacgagttttatcctccgagcagttcggccgcgggcacgatattatgtgcaagttgcggcacaggatcttactgactacggcgaactcagcgactggagtctgcctgcaactgcgaccatgtcactgggaaagggaggagggagtggtggcggcagcggcggaggcagtggcggcggcagccgcaatctgcctgtcgcaactccagatccggggatgttcccgtgtctgcatcatagccaaaatctgcttagggccgtctcaaatatgctccaaaaagcgagacagacgcttgaattttatccgtgcacaagtgaagagattgaccatgaggacatcacgaaggacaaaacgagtacagtggaagcctgtctgcctcttgaactcactaaaaacgaaagctgcctgaatagtcgagaaacttcatttataaccaacggctcatgtcttgcgagccgaaaaacaagttttatgatggctttgtgtctctctagtatttatgaggatctgaaaatgtaccaggttgagttcaagacaatgaacgctaaactccttatggacccgaaacggcagatctttctcgatcaaaacatgctggctgttatcgacgagctcatgcaggcactgaattttaatagcgagaccgtcccgcaaaaaagctccttggaggagccggacttttataagacgaagatcaaactgtgcatcctcctccacgcatttcgcatacgagcggttaccattgaccgggtaatgtcctatctgaatgcaagt

SEQ ID NO: 15 - DNA, 537 bp, Artificial Sequence
  Synthetic polynucleotide
atggctgcgctccaaaaaagtgtgagttcctttttgatgggcacgctcgcaactagctgcttgcttctgctggcgttgctcgtacagggtggtgctgccgcaccaatatcatcccattgccgcctcgacaaaagtaactttcagcagccgtacataactaaccgcaccttcatgctcgcgaaagaagcgagcctcgctgacaacaacacggatgtccgattgattggcgaaaaactgtttcatggagtttccatgagtgaacgatgttatttgatgaaacaagtacttaacttcacattggaagaagttctcttcccacagagtgatcggttccaaccttatatgcaggaggttgtcccttttttggcccgactgtctaataggctttcaacgtgccacattgaaggcgatgaccttcacatacaaaggaatgtgcagaaactgaaagatactgtaaaaaaacttggagagtcaggagaaatcaaagccataggggagctcgatcttcttttcatgtcactgcggaacgcctgtatt

SEQ ID NO: 16 - DNA, 831 bp, Artificial Sequence
  Synthetic polynucleotide
atgataatactgatttatttgttcttgctcctgtgggaggacacgcagggatggggctttaaggacggtatatttcacaatagtatatggctcgaacgagcggcaggcgtttaccatagagaagcacgatctggaaaatataagttgacatacgcagaggcgaaagctgtatgtgagttcgaagggggacatcttgcaacctataaacaattggaggctgcgagaaagatcggattccacgtctgcgctgctgggtggatggccaaaggcagggtaggttaccctatagtcaagcctggtcctaactgtggttttggtaagacagggattatcgactacggtataaggctcaatcgaagcgagagatgggatgcctattgctataatccccacgcgaaagaatgcggcggtgtctttacggacccaaagcagatctttaagagcccaggttttccaaacgagtacgaggataaccaaatatgttattggcacattagattgaaatatgggcagagaatacaccttagttttctcgatttcgatctggaggatgatccagggtgtctggcggattatgttgagatctatgatagctacgatgacgttcacggtttcgttgggagatactgcggggacgaactccccgacgacatcataagtactggtaatgtaatgactctcaaatttctgagcgatgcttcagtgaccgcaggcggattccaaattaagtatgtggcaatggaccccgtatccaaaagcagccagggaaaaaataccagtaccacttccacaggaaacaaaaatttccttgcaggacgctttagtcacttg

SEQ ID NO: 17 - DNA, 1065 bp, Artificial Sequence
  Synthetic polynucleotide
atggcattttcaggatcacaagctccatacttgagcccagcagtgccattttctggcacgattcaaggcggactgcaagacggcttgcagataacagtcaacggaacagtactgtcaagtagcggtacacggttcgcggtgaactttcagactggattttctggcaatgacatcgcattccacttcaatccaaggttcgaagatggaggttatgttgtttgcaatactaggcaaaacggcagttgggggcccgaggagcggaaaacccacatgccattccagaaagggatgccgttcgatctctgctttcttgttcagagttcagatttcaaagttatggtcaatggcatattgttcgtacaatatttccatcgagtgcccttccatagggtcgacactatcagtgtcaacggttctgtccaactttcctatatatccttccagaatcctcgaactgtacctgtgcaaccggcgttttcaaccgtcccgttcagccagcccgtgtgcttccccccgagaccaaggggtaggcgacagaaaccaccgggtgtctggccagcaaacccggcccctatcacgcaaacagtgatacatactgtacagagcgcacctggacaaatgttcagcacacctgccatacctcccatgatgtatccccaccctgcgtaccccatgccattcatcacaaccatactcggcggactgtacccctctaagtccatcctcctttctggtaccgtcctcccgagcgcacagcgattccacatcaatttgtgctctggtaaccatatcgctttccatttgaacccacgattcgacgaaaacgcggtagtaaggaatacacaaattgacaactcttggggtagtgaagaacgctccttgccacggaaaatgccgtttgtccgaggccagagctttagtgtgtggattctctgtgaggcacactgtcttaaggtagccgttgatgggcagcatctctttgaatactatcacaggcttcggaacctcccgacaatcaatcggctggaagttgggggggatatacagttgactcacgtgcaaacc

SEQ ID NO: 18 - DNA, 606 bp, Artificial Sequence
  Synthetic polynucleotide
atgaaagttcttgccgcaggggtggttcctctgttgctcgtcttgcactggaaacacggggcagggagcccgcttcccattacgcctgtgaatgcaacgtgcgcaattaggcatccgtgccataataatctgatgaaccagattaggtcccaactcgcacagctcaatggttcagcgaacgcgctttttatcttgtattatacggcacagggcgaaccgtttccaaataaccttgataaactgtgcgggccgaacgtcaccgacttcccgccattccatgcgaacggcacggagaaagcaaaactcgtagagctgtatcggattgtagtatatctgggcacaagccttggcaacataacacgggaccaaaaaattttgaacccctcagctttgagtctccacagtaaactcaatgcgacagcagatattctgagggggctcctgtcaaatgtgctttgcagactgtgctctaagtaccatgttgggcatgttgacgtaacgtacgggcctgacacttccgggaaagacgtatttcagaaaaagaagctcggctgccaactcctgggcaaatacaagcagatcatagccgttcttgcccaggcgttc

SEQ ID NO: 19 - DNA, 957 bp, Artificial Sequence
  Synthetic polynucleotide
atggtggttatggcaccaaggactctctttttgttgctcagcggggcgttgactctcacagaaacgtgggctggtagccattctatgcgatatttcagcgccgcagtgtcaagaccggggcggggtgaaccgagatttatagctatgggttacgtggatgacacccagtttgtgcggttcgatagtgattctgcgtgcccaaggatggaaccccgcgcaccgtgggttgaacaagagggtcccgaatactgggaagaagagactcgaaatacaaaagcacacgcccaaaccgacagaatgaacttgcagactttgcgaggatactataatcagagcgaggcaagcagtcatacccttcagtggatgattgggtgcgatcttggctcagacggacggctcctgcgggggtatgaacagtatgcttacgatggaaaagactacctggctctgaacgaggatctgaggtcctggacagctgccgataccgctgcacagatatctaagagaaaatgcgaggcggccaatgtcgccgaacagcgcagagcgtatttggagggaacgtgcgtagagtggctccacagatatctggagaacggaaaagaaatgcttcaacgcgcggaccctcctaaaacccacgtgactcatcatcctgttttcgattatgaggccacgctgagatgttgggctttgggattttatcctgcggaaatcatccttacctggcagcgagatggtgaggaccagacccaagatgtcgaattggtggaaacacgaccagcaggtgacggcacgtttcaaaaatgggcggccgtagtggtaccttccggagaggagcagcgatatacttgtcacgttcaacatgagggactccctgagccactgatgctgaggtggtccaaggaaggagacggtggcataatgtcagtccgcgagagccgatctctttccgaagatctg

SEQ ID NO: 20 - DNA, 56 bp, Artificial Sequence
  Synthetic polynucleotide
gggtcttcgggaagtgagcagaagctgatcagcgaggaggacctgtaagaagactg

SEQ ID NO: 21 - protein, 290 aa, Artificial Sequence
  Synthetic polypeptide
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 22 - protein, 178 aa, Artificial Sequence
  Synthetic polypeptide
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 23 - protein, 199 aa, Artificial Sequence
  Synthetic polypeptide
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 24 - protein, 146 aa, Artificial Sequence [sequence omitted]

SEQ ID NO: 25 - protein, 153 aa, Artificial Sequence [sequence omitted]

SEQ ID NO: 26 - protein, 442 aa, Artificial Sequence
  Synthetic polypeptide
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGKGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 27 - protein, 179 aa, Artificial Sequence
  Synthetic polypeptide
MAALQKSVSSFLMGTLATSCLLLLALLVQGGAAAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 28 - protein, 277 aa, Artificial Sequence
  Synthetic polypeptide
MIILIYLFLLLWEDTQGWGFKDGIFHNSIWLERAAGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKECGGVFTDPKQIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTSTTSTGNKNFLAGRFSHL

SEQ ID NO: 29 - protein, 355 aa, Artificial Sequence
  Synthetic polypeptide
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 30 - protein, 202 aa, Artificial Sequence
  Synthetic polypeptide
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 31 - protein, 319 aa, Artificial Sequence
  Synthetic polypeptide
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWSKEGDGGIMSVRESRSLSEDL

SEQ ID NO: 32 - protein, 177 aa, Artificial Sequence
  Synthetic polypeptide
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 33 - protein, 217 aa, Artificial Sequence
  Synthetic polypeptide
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDGSNRWMFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 453 aa, Artificial Sequence
  Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGSSGWYVPHWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 214 aa, Artificial Sequence
  Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 226 aa, Artificial Sequence
  Synthetic polypeptide
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTH

SEQ ID NO: 37 - protein, 257 aa, Artificial Sequence
  Synthetic polypeptide
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 38 - protein, 124 aa, Artificial Sequence
  Synthetic polypeptide
QVQLQDSGGGLVQAGGSLRLSCAASGGTFSSIIMAWFRQAPGKEREFVGAVSWSGGTTVYADSVLGRFEISRDSARKSVYLQMNSLKPEDTAVYYCAARPYQKYNWASASYNVWGQGTQVTVSS

SEQ ID NO: 39 - protein, 214 aa, Artificial Sequence
  Synthetic polypeptide
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 224 aa, Artificial Sequence
  Synthetic polypeptide
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 41 - protein, 217 aa, Artificial Sequence
  Synthetic polypeptide
QSVLTQPPSVSGAPGQRVTISCTGSSSNTGAGYDVHWYQQVPGTAPKLLIYGSGNRPSGVPDRFSGSKSGTSASLAITGLQAEDEADYYCQSYDSSLSGWVFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - protein, 450 aa, Artificial Sequence
  Synthetic polypeptide
QVQLVESGGGVVQPGRSLRLSCAASGFTFSSYGMHWVRQAPGKGLEWVAVIWYDGSNEYYADSVKGRFTISRDNSKNTLYLQMNSLRAEDTAVYYCARDRGYTSSWYPDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 43 - protein, 403 aa, Artificial Sequence [sequence omitted]

SEQ ID NO: 44 - protein, 1153 aa, Artificial Sequence
  Synthetic polypeptide
MACPWKFLFKTKFHQYAMNGEKDINNNVEKAPCATSSPVTQDDLQYHNLSKQQNESPQPLVETGKKSPESLVKLDATPLSSPRHVRIKNWGSGMTFQDTLHHKAKGILTCRSKSCLGSIMTPKSLTRGPRDKPTPPDELLPQAIEFVNQYYGSFKEAKIEEHLARVEAVTKEIETTGTYQLTGDELIFATKQAWRNAPRCIGRIQWSNLQVFDARSCSTAREMFEHICRHVRYSTNNGNIRSAITVFPQRSDGKHDFRVWNAQLIRYAGYQMPDGSIRGDPANVEFTQLCIDLGWKPKYGRFDVVPLVLQANGRDPELFEIPPDLVLEVAMEHPKYEWFRELELKWYALPAVANMLLEVGGLEFPGCPFNGWYMGTEIGVRDFCDVQRYNILEEVGRRMGLETHKLASLWKDQAVVEINIAVLHSFQKQNVTIMDHHSAAESFMKYMQNEYRSRGGCPADWIWLVPPMSGSITPVFHQEMLNYVLSPFYYYQVEAWKTHVWQDEKRRPKRREIPLKVLVKAVLFACMLMRKTMASRVRVTILFATETGKSEALAWDLGALFSCAFNPKVVCMDKYRLSCLEEERLLLVVTSTFGNGDCPGNGEKLKKSLFMLKELNNKFRYAVFGLGSSMYPRFCAFAHDIDQKLSHLGASQLTPMGEGDELSGQEDAFRSWAVQTFKAACETFDVRGKQHIQIPKLYTSNVTWDPHHYRLVQDSQPLDLSKALSSMHAKNVFTMRLKSRQNLQSPTSSRATILVELSCEDGQGLNYLPGEHLGVCPGNQPALVQGILERVVDGPTPHQTVRLEALDESGSYWVSDKRLPPCSLSQALTYFLDITTPPTQLLLQKLAQVATEEPERQRLEALCQPSEYSKWKFTNSPTFLEVLEEFPSLRVSAGFLLSQLPILKPRFYSISSSRDHTPTEIHLTVAVVTYHTRDGQGPLHHGVCSTWLNSLKPQDPVPCFVRNASGFHLPEDPSHPCILIGPGTGIAPFRSFWQQRLHDSQHKGVRGGRMTLVFGCRRPDEDHIYQEEMLEMAQKGVLHAVHTAYSRLPGKPKVYVQDILRQQLASEVLRVLHKEPGHLYVCGDVRMARDVAHTLKQLVAAKLKLNEEQVEDYFFQLKSQKRYHEDIFGAVFPYEAKKDRVAVQPSSLEMSAL

SEQ ID NO: 45 - protein, 604 aa, Artificial Sequence
  Synthetic polypeptide
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 46 - protein, 288 aa, Artificial Sequence
  Synthetic polypeptide
MERPQPDSMPQDLSEALKEATKEVHTQAENAEFMRNFQKGQVTRDGFKLVMASLYHIYVALEEEIERNKESPVFAPVYFPEELHRKAALEQDLAFWYGPRWQEVIPYTPAMQRYVKRLHEVGRTEPELLVAHAYTRYLGDLSGGQVLKKIAQKALDLPSSGEGLAFFTFPNIASATKFKQLYRSRMNSLEMTPAVRQRVIEEAKTAFLLNIQLFEELQELLTHDTKDQSPSRAPGLRQRASNKVQDSAPVETPRGKPPLNTRSQAPLLRWVLTLSFLVATVAVGLYAM

SEQ ID NO: 47 - protein, 869 aa, Artificial Sequence
  Synthetic polypeptide
MTADKEKKRSSSERRKEKSRDAARCRRSKETEVFYELAHELPLPHSVSSHLDKASIMRLAISFLRTHKLLSSVCSENESEAEADQQMDNLYLKALEGFIAVVTQDGDMIFLSENISKFMGLTQVELTGHSIFDFTHPCDHEEIRENLSLKNGSGFGKKSKDMSTERDFFMRMKCTVTNRGRTVNLKSATWKVLHCTGQVKVYNNCPPHNSLCGYKEPLLSCLIIMCEPIQHPSHMDIPLDSKTFLSRHSMDMKFTYCDDRITELIGYHPEELLGRSAYEFYHALDSENMTKSHQNLCTKGQVVSGQYRMLAKHGGYVWLETQGTVIYNPRNLQPQCIMCVNYVLSEIEKNDVVFSMDQTESLFKPHLMAMNSIFDSSGKGAVSEKSNFLFTKLKEEPEELAQLAPTPGDAIISLDFGNQNFEESSAYGKAILPPSQPWATELRSHSTQSEAGSLPAFTVPQAAAPGSTTPSATSSSSSCSTPNSPEDYYTSLDNDLKIEVIEKLFAMDTEAKDQCSTQTDFNELDLETLAPYIPMDGEDFQLSPICPEERLLAENPQSTPQHCFSAMTNIFQPLAPVAPHSPFLLDKFQQQLESKKTEPEHRPMSSIFFDAGSKASLPPCCGQASTPLSSMGGRSNTQWPPDPPLHFGPTKWAVGDQRTEFLGAAPLGPPVSPPHVSTFKTRSAKGFGARGPDVLSPAMVALSNKLKLKRQLEYEEQAFQDLSGGDPPGGSTSHLMWKRMKNLRGGSCPLMPDKPLSANVPNDKFTQNPMRGLGHPLRHLPLPQPPSAISPGENSKSRFPPQCYATQYQDYSLSSAHKVSGMASLLGPSFESYLLPELTRYDCEVNVPVLGSSTLLQGGDLLRALDQAT

SEQ ID NO: 48 - protein, 15 aa, Artificial Sequence
  Synthetic polypeptide
GSSGSEQKLISEEDL

SEQ ID NO: 49 - DNA, 6029 bp, Artificial Sequence
  Synthetic polynucleotide
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaatcaaaattttatctcgacatggtggcgaccggtagcgctagcggatcgataagcttgatatcgcctgcagccgaattccttgacttgggatccgcgtcaagtggagcaaggcaggtggacagtcctgcaggcatgcgtgactgactgaggccgcgactctagtttaaactgcgtgactgactctagaagatccggcagtgcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg